SEQUENCE LISTING (28 sequences)

SEQ ID NO: 1 - DNA, 22 bp, Artificial Sequence
  miR-183 target
agtgaattctaccagtgccata

SEQ ID NO: 2 - DNA, 23 bp, Artificial sequence
  mirR-96 target
agcaaaaatgtgctagtgccaaa

SEQ ID NO: 3 - DNA, 24 bp, Artificial sequence
  miR-182 target
agtgtgagttctaccattgccaaa

SEQ ID NO: 4 - DNA, 23 bp, Artificial sequence
  miR-145 target
agggattcctgggaaaactggac

SEQ ID NO: 5 - DNA, 4 bp, Artificial sequence
  Spacer (i)
ggat

SEQ ID NO: 6 - DNA, 6 bp, Artificial sequence
  Spacer (ii)
cacgtg

SEQ ID NO: 7 - DNA, 6 bp, Artificial sequence
  spacer iii
gcatgc

SEQ ID NO: 8 - DNA, 2211 bp, adeno-associated virus hu68
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 9 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - DNA, 3198 bp, Artificial sequence
  ITR.CB7.CI.eGFP.miR145.rBG.ITR
  repeat_region (1)..(130): 5' - AAV2 - ITR
  misc_feature (1)..(130): 5' - AAV2 - ITR
  promoter (198)..(579): CMV IE promoter
  promoter (582)..(863): CB promoter
  misc_feature (1979)..(2698): eGFP gene
  misc_feature (2705)..(2727): miR145
  misc_feature (2728)..(2731): spacer
  misc_feature (2732)..(2754): miR145
  misc_feature (2755)..(2760): spacer
  misc_feature (2761)..(2783): miR145
  misc_feature (2784)..(2789): spacer
  misc_feature (2790)..(2812): miR145
  misc_feature (2981)..(3198): 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagggattcctgggaaaactggacggatagggattcctgggaaaactggaccacgtgagggattcctgggaaaactggacgcatgcagggattcctgggaaaactggacgcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 3202 bp, Artificial sequence
  ITR.CB7.CI.eGFP.miR182.rGB.ITR
  misc_feature (1)..(130): 5' ITR (AAV2)
  misc_feature (198)..(579): CMV IE promoter
  misc_feature (582)..(863): CB promoter
  misc_feature (958)..(1930): chicken beta-actin intron
  misc_feature (1979)..(2698): eGFP coding sequence
  misc_feature (2705)..(2728): miR182
  misc_feature (2729)..(2732): spacer
  misc_feature (2733)..(2756): miR182
  misc_feature (2757)..(2760): spacer
  misc_feature (2763)..(2786): miR182
  misc_feature (2787)..(2792): spacer
  misc_feature (2793)..(2816): miR182
  polyA_signal (2858)..(2984)
  misc_feature (3073)..(3202): 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgtgagttctaccattgccaaaggatagtgtgagttctaccattgccaaacacgtgagtgtgagttctaccattgccaaagcatgcagtgtgagttctaccattgccaaagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 3198 bp, Artificial sequence
  ITR.CB7.eGFP.miRNA96.rBG.ITR
  misc_feature (1979)..(2699): eGFP coding sequence
  misc_feature (2705)..(2727): miR96
  misc_feature (2728)..(2731): spacer
  misc_feature (2732)..(2754): miR96
  misc_feature (2755)..(2760): spacer
  misc_feature (2761)..(2783): miR96
  misc_feature (2784)..(2789): spacer
  misc_feature (2790)..(2812): miR96
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagcaaaaatgtgctagtgccaaaggatagcaaaaatgtgctagtgccaaacacgtgagcaaaaatgtgctagtgccaaagcatgcagcaaaaatgtgctagtgccaaagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 3194 bp, Artificial sequence
  ITR.CB7.CI.eGFP.miRNA183.rBG.ITR
  misc_feature (1979)..(2698): eGFP coding sequence
  misc_feature (2705)..(2726): miRNA183
  misc_feature (2727)..(2730): spacer
  misc_feature (2731)..(2752): miRNA183
  misc_feature (2753)..(2758): spacer
  misc_feature (2781)..(2786): spacer
  misc_feature (2787)..(2808): miRNA183
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgaattctaccagtgccataggatagtgaattctaccagtgccatacacgtgagtgaattctaccagtgccatagcatgcagtgaattctaccagtgccatagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 4300 bp, Artificial sequence
  ITR.CB7.CI.hIDUAcoV1.rBG.ITR
  misc_feature (1)..(130): 5' ITR
  misc_feature (198)..(579): CMV IE promoter
  misc_feature (582)..(863): CB promoter
  misc_feature (836)..(839): TATA
  misc_feature (958)..(1930): chicken beta-actin intron
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagccaccatgaggcctctcagacctagagctgctctgctggcactgctggcttctctgcttgctgctcctcctgtggctcctgccgaagctcctcatctggtgcacgtggatgccgccagagcactgtggcccctgagaagattttggcggagcaccggcttttgccctccactgcctcattctcaggccgaccagtacgtgctgagctgggaccagcaactgaacctggcctacgtgggagccgtgcctcacagaggcattaagcaagtgcggacccactggctgctggaactggtcacaacaagaggcagcacaggcagaggcctgagctacaacttcacccacctggacggctacctggacctgctgagagagaatcagctgctgcctggcttcgagctgatgggctctgcctctggccacttcaccgacttcgaggacaagcagcaggttttcgagtggaaggacctggtgtccagcctggccagacggtacatcggcagatacggactggcccacgtgtccaagtggaacttcgagacctggaacgagcccgaccaccacgacttcgacaacgtgtcaatgaccatgcagggctttctgaactactacgacgcctgcagcgagggcctgagagctgcttctcctgctctgagacttggcggccctggcgactcttttcacacccctccaagaagccctctgtcctggggactgctgagacactgtcacgacggcaccaatttcttcaccggcgaggctggcgtgcggctggattatatcagcctgcacagaaagggcgccagaagcagcatcagcatcctggaacaagagaaggtggtggcccagcagatcagacagctgttccccaagttcgccgacacacccatctacaacgacgaggccgatcctctcgttggctggtcacttcctcagccttggagagccgatgtgacctatgccgccatggtggtcaaagtgatcgcccagcaccagaatctgctgctcgccaataccaccagcgcctttccatacgctctgctgagcaacgacaacgccttcctgagctatcaccctcatcctttcgctcagcggaccctgaccgccagattccaagtgaacaacacccggcctccacacgtgcagctgctgagaaaaccagtgctgacagccatgggcctgctcgccctgctggacgaagaacaactgtgggccgaagtgtcccaggccggaacagtgctggatagcaatcacacagtgggcgtgctggcctccgctcatagacctcaaggaccagccgatgcttggagggctgccgtgctgatctacgccagcgacgatacaagggctcaccccaacagatccgtggccgtgacactgagactgagaggcgttccaccaggacctggcctggtgtacgtgaccagatacctggacaacggcctgtgcagccctgatggcgaatggcgtagactaggcagacctgtgtttcctaccgccgagcagttcagacggatgagagccgctgaagatcccgtggctgctgctccaagacctcttccagctggtggcagactgactctgaggcctgcactcagactgcctagtctgctgctggtgcacgtctgtgccagacctgagaagcctcctggccaagtgacaagactgagggccctgccactgacacagggacagctggttcttgtttggagcgacgagcacgtgggcagcaagtgtctgtggacctacgagatccagttcagccaggacggcaaggcctacacacccgtgtctagaaagcctagcaccttcaacctgttcgtgttcagccccgatacaggcgccgtgtctggcagctatagagtcagagccctggactactgggccagaccaggaccattttctgaccccgtgccttacctggaagtgcccgttcctagaggccctccttctcctggaaatccctgataaggtaccatacctaggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 4403 bp, Artificial sequence
  ITR.CB7.CI.hIDUAcoV1.miR183.ITR
  misc_feature (1)..(130): 5' ITR
  misc_feature (198)..(579): CMV IE promoter
  misc_feature (582)..(863): CB promoter
  misc_feature (1938)..(3908): hIDUAcoV1
  misc_feature (3915)..(3936): miRNA183
  misc_feature (3937)..(3940): spacer
  misc_feature (3941)..(3962): miRNA183
  misc_feature (3963)..(3968): spacer
  misc_feature (3969)..(3990): miRNA183
  misc_feature (3991)..(3996): spacer
  misc_feature (3997)..(4018): miRNA183
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagccaccatgaggcctctcagacctagagctgctctgctggcactgctggcttctctgcttgctgctcctcctgtggctcctgccgaagctcctcatctggtgcacgtggatgccgccagagcactgtggcccctgagaagattttggcggagcaccggcttttgccctccactgcctcattctcaggccgaccagtacgtgctgagctgggaccagcaactgaacctggcctacgtgggagccgtgcctcacagaggcattaagcaagtgcggacccactggctgctggaactggtcacaacaagaggcagcacaggcagaggcctgagctacaacttcacccacctggacggctacctggacctgctgagagagaatcagctgctgcctggcttcgagctgatgggctctgcctctggccacttcaccgacttcgaggacaagcagcaggttttcgagtggaaggacctggtgtccagcctggccagacggtacatcggcagatacggactggcccacgtgtccaagtggaacttcgagacctggaacgagcccgaccaccacgacttcgacaacgtgtcaatgaccatgcagggctttctgaactactacgacgcctgcagcgagggcctgagagctgcttctcctgctctgagacttggcggccctggcgactcttttcacacccctccaagaagccctctgtcctggggactgctgagacactgtcacgacggcaccaatttcttcaccggcgaggctggcgtgcggctggattatatcagcctgcacagaaagggcgccagaagcagcatcagcatcctggaacaagagaaggtggtggcccagcagatcagacagctgttccccaagttcgccgacacacccatctacaacgacgaggccgatcctctcgttggctggtcacttcctcagccttggagagccgatgtgacctatgccgccatggtggtcaaagtgatcgcccagcaccagaatctgctgctcgccaataccaccagcgcctttccatacgctctgctgagcaacgacaacgccttcctgagctatcaccctcatcctttcgctcagcggaccctgaccgccagattccaagtgaacaacacccggcctccacacgtgcagctgctgagaaaaccagtgctgacagccatgggcctgctcgccctgctggacgaagaacaactgtgggccgaagtgtcccaggccggaacagtgctggatagcaatcacacagtgggcgtgctggcctccgctcatagacctcaaggaccagccgatgcttggagggctgccgtgctgatctacgccagcgacgatacaagggctcaccccaacagatccgtggccgtgacactgagactgagaggcgttccaccaggacctggcctggtgtacgtgaccagatacctggacaacggcctgtgcagccctgatggcgaatggcgtagactaggcagacctgtgtttcctaccgccgagcagttcagacggatgagagccgctgaagatcccgtggctgctgctccaagacctcttccagctggtggcagactgactctgaggcctgcactcagactgcctagtctgctgctggtgcacgtctgtgccagacctgagaagcctcctggccaagtgacaagactgagggccctgccactgacacagggacagctggttcttgtttggagcgacgagcacgtgggcagcaagtgtctgtggacctacgagatccagttcagccaggacggcaaggcctacacacccgtgtctagaaagcctagcaccttcaacctgttcgtgttcagccccgatacaggcgccgtgtctggcagctatagagtcagagccctggactactgggccagaccaggaccattttctgaccccgtgccttacctggaagtgcccgttcctagaggccctccttctcctggaaatccctgataaggtaccagtgaattctaccagtgccataggatagtgaattctaccagtgccatacacgtgagtgaattctaccagtgccatagcatgcagtgaattctaccagtgccatagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 2211 bp, adeno-associated virus 9
  CDS (1)..(2211)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 17 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - DNA, 1962 bp, Homo sapiens
  CDS (1)..(1962)
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 19 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 20 - DNA, 3106 bp, Artificial sequence
  CB7.CI.eGFP.miR183(1x).RBG
  repeat_region (1)..(130): AAV2 - 5' ITR
  enhancer (198)..(579): human cytomegalovirus (CMV) immediate early (IE) enhancer
  promoter (582)..(862): chicken beta-actin (CB) promoter
  Intron (956)..(1928): chicken beta-actin intron
  Misc (1977)..(2696): eGFP coding sequence
  misc_RNA (2699)..(2720): miR183
  polyA_signal (2762)..(2888): rabbit globin polyA
  repeat_region (2977)..(3106): AAV2 - 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcagtgaattctaccagtgccatagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 3136 bp, Artificial sequence
  CB7.CI.eGFP.miRNA183(2x).RBG
  repeat_region (1)..(130): AAV 2 - 5' ITR
  enhancer (198)..(579): hCMV IE enhancer
  promoter (582)..(862): CB promoter
  Intron (956)..(1928): chicken beta-actin intron
  misc_feature (1977)..(2696): eGFP coding sequence
  misc_RNA (2701)..(2722): miRNA183
  misc_feature (2701)..(2722): spacer
  misc_feature (2723)..(2728): spacer
  misc_RNA (2729)..(2750): miRNA183
  polyA_signal (2792)..(2916): rabbit globin polyA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtgagtgaattctaccagtgccatagcatgcagtgaattctaccagtgccatagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 3163 bp, Artificial sequence
  CB7.CI.eGFP.miRNA183(3x).RBG
  repeat_region (1)..(130): AAV2 - 5' ITR
  enhancer (198)..(579): CMV IE enhancer
  promoter (582)..(862): CB promoter
  Intron (956)..(1928): chicken beta-actin promoter
  misc_feature (1977)..(2696): eGFP coding sequence
  misc_RNA (2703)..(2724): miRNA183
  misc_feature (2725)..(2728): spacer
  misc_RNA (2729)..(2750): miRNA183
  misc_feature (2751)..(2756): spacer
  misc_RNA (2757)..(2778): miRNA183
  polyA_signal (2819)..(2945): rabbit globin polyA
  repeat_region (3034)..(3163)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgaattctaccagtgccataggatagtgaattctaccagtgccatacacgtgagtgaattctaccagtgccatagggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 3192 bp, Artificial Sequence
  CB7.CI.eGFP.miRNA183.RBG
  repeat_region (1)..(130): AAV2 = 5' ITR
  enhancer (198)..(579): hCMV IE enhancer
  promoter (582)..(862): CB promoter
  Intron (956)..(1926): chicken betan-actin intron
  misc_feature (1977)..(2696): eGFP coding sequence
  misc_RNA (2703)..(2724): miRNA183
  misc_feature (2725)..(2729): spacer
  misc_RNA (2729)..(2750): miRNA183
  misc_RNA (2757)..(2778): miRNA183
  misc_feature (2779)..(2784): spacer
  misc_RNA (2785)..(2806): miRNA183
  polyA_signal (2848)..(2974): rabbit globin polyA
  repeat_region (3063)..(3192): AAV2 - 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgaattctaccagtgccataggatagtgaattctaccagtgccatacacgtgagtgaattctaccagtgccatagcatgcagtgaattctaccagtgccatagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - protein, 9 aa, Artificial sequence
  LAGLIDADG
LAGLIDADG

SEQ ID NO: 25 - DNA, 22 bp, Artificial sequence
  endonuclease recognition site
caaaacgtcgtgagacagtttg

SEQ ID NO: 26 - DNA, 3283 bp, Artificial Sequence
  synthetic construct
  repeat_region (1)..(130): 5' ITR
  enhancer (198)..(579): CMV IE enhancer
  promoter (582)..(862): CB promoter
  TATA_signal (836)..(839): TATA
  Intron (956)..(1928): chicken beta-actin intron
  misc_feature (1977)..(2696): eGFP
  misc_feature (2729)..(2760): miR183
  misc_feature (2757)..(2778): miR183
  misc_feature (2785)..(2806): miR183
  misc_feature (2815)..(2836): miR183
  misc_feature (2858)..(2879): miR183
  misc_feature (2884)..(2905): miR183
  polyA_signal (2939)..(3065): Rabbit globin poly A
  repeat_region (3154)..(3283): 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgaattctaccagtgccataggatagtgaattctaccagtgccatacacgtgagtgaattctaccagtgccatagcatgcagtgaattctaccagtgccatagcggccgcagtgaattctaccagtgccatatcacagtgaattctaaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 27 - DNA, 3294 bp, Artificial Sequence
  synthetic construct
  repeat_region (1)..(130): 5' ITR
  enhancer (198)..(579): CMV IE enhancer
  promoter (582)..(862): CB promoter
  TATA_signal (836)..(839)
  Intron (956)..(1928): chicken beta-actin intron
  misc_feature (1977)..(2696): eGFP
  misc_feature (2703)..(2724): miR183
  misc_feature (2729)..(2750): miR183
  misc_feature (2757)..(2778): miR183
  misc_feature (2785)..(2806): miR183
  misc_feature (2815)..(2836): miR183
  misc_feature (2841)..(2862): miR183
  misc_feature (2869)..(2890): miR183
  misc_feature (2895)..(2916): miR183
  polyA_signal (2950)..(3076): Rabbit globin poly A
  repeat_region (3165)..(3294): 3 ' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgaattctaccagtgccataggatagtgaattctaccagtgccatacacgtgagtgaattctaccagtgccatagcatgcagtgaattctaccagtgccatagcggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 3304 bp, Artificial Sequence
  synthetic construct
  repeat_region (1)..(130): 5' ITR
  enhancer (198)..(579): CMV IE enhancer
  promoter (582)..(863): CB promoter
  TATA_signal (836)..(839): TATA
  misc_feature (836)..(2698): eGFP
  Intron (958)..(1930)
  misc_feature (2705)..(2728): miR182
  misc_feature (2733)..(2756): miR182
  misc_feature (2763)..(2786): miR182
  misc_feature (2793)..(2816): miR182
  misc_feature (2825)..(2846): miR183
  misc_feature (2851)..(2872): miR183
  misc_feature (2879)..(2900): miR183
  misc_feature (2905)..(2926): miR183
  polyA_signal (2960)..(3086): Rabbit globin poly A
  repeat_region (3175)..(3304): 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgtgagttctaccattgccaaaggatagtgtgagttctaccattgccaaacacgtgagtgtgagttctaccattgccaaagcatgcagtgtgagttctaccattgccaaagcggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2023/0304034 A1
(43) Pub. Date: Sep. 28, 2023

(54) COMPOSITIONS FOR DRG-SPECIFIC REDUCTION OF TRANSGENE EXPRESSION

(71) Applicant: The Trustees of the University of Pennsylvania, Philadelphia, PA (US)

(72) Inventors: Juliette Hordeaux, Philadelphia, PA (US); James M. Wilson, Philadelphia, PA (US)

(21) Appl. No.: 17/998,328

(22) PCT Filed: May 12, 2021
(86) PCT No.: PCT/US2021/032003

§ 371 Date: Nov. 9, 2022

Related U.S. Application Data
(60) Provisional application No. 63/152,042, filed on Feb. 22, 2021.
(60) Provisional application No. 63/079,299, filed on Sep. 16, 2020.
(60) Provisional application No. 63/043,562, filed on Jun. 24, 2020.
(60) Provisional application No. 63/038,488, filed on Jun. 12, 2020.
(60) Provisional application No. 63/023,593, filed on May 12, 2020.

(51) Int. Cl. C12N 15/86; C12N 15/113; A61K 48/00; A61K 9/00

(52) U.S. Cl. CPC C12N 15/86; A61K 9/0019; A61K 48/0066; C12N 15/113; C12N 2310/141; C12N 2750/14143

(57) ABSTRACT

A recombinant AAV (rAAV) for delivery of a gene product to a patient in need thereof which specifically represses expression of the gene product in dorsal root ganglia (DRG) is provided. The rAAV comprises an AAV capsid having packaged therein a vector genome, wherein the vector genome comprises: (a) a coding sequence for the gene product under the control of regulatory sequences that direct expression of the gene product in a cell containing the vector genome; and (b) at least eight miR target sequences, wherein each target sequence is specific for miR-183 or miR-182, and wherein the at least eight miR target sequences are operably linked to the 3′ end of the coding sequence. Also provided are methods and uses of the described rAAVs for delivery of a gene product to a patient in need thereof.

### BACKGROUND OF THE INVENTION

The vector platform of choice for in vivo gene therapy is based on primate-derived adeno-associated viruses (AAV). In the 1960s, gene-therapy products were derived from AAVs isolated from preparations of adenoviruses (Hoggan, M.D. et al. Proc Natl Acad Sci U S A 55:1467-1474, 1966). Although these vectors were safe, many programs failed in the clinic because of poor transduction. At the turn of the century, researchers discovered a family of endogenous AAVs that, as vectors, achieved much higher transduction efficiencies while retaining favorable safety profiles (Gao, G., et al. J Virol 78:6381-6388, 2004).

Untoward responses of the host to AAV vectors have been minimal. In contrast to non-viral and adenoviral vectors, which elicit vibrant acute inflammatory responses (Raper, S.E., et al. Mol Genet Metab 80:148-158, 2003; Zhang, Y., et al. Mol Ther 3:697-707, 2001), AAV vectors are not pro-inflammatory. Destructive adaptive immune responses to vector-transduced cells—such as cytotoxic T cells—have been minimal following AAV vector administration. There is evidence in animals and humans that AAV can induce tolerance to capsid or transgene products under certain circumstances depending on the serotype, dose, route of administration, and immune-suppression regimen (Gernoux, G., et al. Hum Gene Ther 28:338-349, 2017; Mays, L.E. & Wilson, J.M. Mol Ther 19:16-27, 2011; Manno, C.S., et al. Nat Med 12:342-347, 2006; Mingozzi, F., et al. Blood 110:2334-2341, 2007). However, given the current expansion of clinical applications of AAV gene therapy, we are beginning to see toxicities that can limit the clinical impact of this technology.

The most severe toxicities have occurred following intravenous administration of high doses of AAV to target the CNS and musculoskeletal system. Studies in nonhuman primates (NHPs) showed the acute development of thrombocytopenia and transaminitis, which, in some cases, evolved into a lethal syndrome of hemorrhage and shock (Hordeaux, J., et al. Mol Ther 26:664-668, 2018; Hinderer, C., et al. Hum Gene Ther. 29(3):285-298, 2018). Acute elevations in liver enzymes and/or reductions in platelets have also been observed in most high-dose AAV clinical trials (AveXis, I. ZOLGENSMA Prescribing Information, 2019; Solid Biosciences Provides SGT-001 Program Update, 2019; Pfizer, Pfizer Presents Initial Clinical Data on Phase 1b Gene Therapy Study for Duchenne Muscular Dystrophy (DMD), 2019; Flanigan, K.T. et al. Molecular Genetics and Metabolism 126:S54, 2019). Although infrequent, severe toxicities were characterized by anemia, renal failure, and complement activation (Solid Biosciences, 2019; Pfizer, 2019).

More recently, the problem of degenerating neurons in the dorsal root ganglia (DRG) of NHPs and pigs that received AAV vector either into the cerebral spinal fluid (CSF) or at high doses into the blood has been observed (Hinderer, C., et al. Hum Gene Ther. 29(3):285-298, 2018; Hordeaux, J., et al. Mol Ther Methods Clin Dev 10:68-78, 2018; Hordeaux, J., et al. Mol Ther Methods Clin Dev 10:79-88, 2018). This neuronal toxicity is associated with degeneration of both the peripheral axons in peripheral nerves and the central axons that ascend through the dorsal columns of the spinal cord.

A need in the art exists for compositions and methods for gene therapy which minimize expression of a gene product in cells that are more sensitive to toxicity.

### SUMMARY OF THE INVENTION

In one aspect, provided herein is a recombinant AAV (rAAV) for delivery of a gene product to a patient in need thereof which specifically represses expression of the gene product in dorsal root ganglia (DRG). The rAAV comprises an AAV capsid having packaged therein a vector genome, wherein the vector genome comprises: (a) a coding sequence for the gene product under the control of regulatory sequences that direct expression of the gene product in a cell containing the vector genome; and (b) at least eight miR target sequences, wherein each target sequence is specific for miR-183 or miR-182, and wherein the at least eight miR target sequences are operably linked to the 3′ end of the coding sequence. In certain embodiments, the at least eight miR target sequences comprise at least five, at least six, at least seven, or at least eight target sequences specific for miR-183. In certain embodiments, the at least eight miR target sequences comprise at least five, at least six, at least seven, or at least eight target sequences specific for miR-182. In certain embodiments, the at least eight miR target sequences comprise at least four target sequences specific for miR-183 and/or at least four target sequences specific for miR-182. In certain embodiments, the at least eight miR target sequences comprise four target sequences specific for miR-183 and four target sequences specific for miR-182.

In one aspect, provided herein is a composition for gene delivery which specifically represses expression of a gene product in dorsal root ganglia (DRG), comprising an expression cassette that is a nucleic acid sequence comprising: (a) a coding sequence for the gene product under the control of regulatory sequences that direct expression of the gene product in a cell containing the expression cassette; and (b) at least eight miR target sequences, wherein each target sequence is specific for miR-183 or miR-182, and wherein the at least eight miR target sequences are operably linked to the 3′ end of the coding sequence. In certain embodiments, the at least eight miR target sequences comprise at least five, at least six, at least seven, or at least eight target sequences specific for miR-182. In certain embodiments, the at least eight miR target sequences comprise at least four target sequences specific for miR-183 and/or at least four target sequences specific for miR-182. In certain embodiments, the at least eight miR target sequences comprise four target sequences specific for miR-183 and four target sequences specific for miR-182. In certain embodiments, the expression cassette is carried by a viral vector that is a recombinant parvovirus, a recombinant lentivirus, a recombinant retrovirus, or a recombinant adenovirus. In other embodiments the expression cassette is carried by a non-viral vector that is naked DNA, naked RNA, an inorganic particle, a lipid particle, a polymer-based vector, or a chitosan-based formulation.

In one aspect, provided herein is a pharmaceutical composition comprising an rAAV or an expression cassette and a formulation buffer suitable for delivery via intracerebroventricular, intrathecal, intracisternal, or intravenous injection.

In one aspect, provided herein is a method for repressing expression of a gene product in DRG neurons in a patient, wherein the method comprises delivering an rAAV, a composition comprising an expression cassette, or a pharmaceutical composition described herein.

In one aspect, provided herein is a method for modulating neuronal degeneration and/or decreasing secondary dorsal spinal cord axonal degeneration following intrathecal or systemic gene therapy administration to a patient, wherein the method comprises delivering an rAAV, a composition comprising an expression cassette, or a pharmaceutical composition described herein.

In one aspect, provided here is an rAAV, a composition comprising an expression cassette, or a pharmaceutical composition for use in gene delivery, wherein expression of the delivered gene product is repressed in DRG neurons of the patient.

In one aspect, the use an rAAV, a composition comprising an expression cassette, or a pharmaceutical composition for delivering a transgene to a patient is provided wherein, expression of the delivered transgene is repressed in DRG neurons of the patient.

Other aspects and advantages of the invention will be readily apparent from the following detailed description of the invention.

### BRIEF DESCRIPTION OF THE DRAWINGS

FIGS. 1A - 1C show DRG toxicity and secondary axonopathy after AAV ICM administration. (FIG. 1A) DRG contain the cell bodies of sensory pseudo-unipolar neurons, which relay sensory messages from the periphery to the CNS through peripheral axons located in peripheral nerves and central axons located in the ascending dorsal white matter tracts of the spinal cord. (FIG. 1B) Axonopathy and DRG neuronal degeneration. Axonopathy (upper left) manifests as clear vacuoles that are either empty (missing axon) of filled with macrophages digesting myelin and cellular debris (arrow). DRG lesions (upper right and lower left) consist of neuronal cell-body degeneration (arrow) with mononuclear cell infiltrate (circle). An eosinophilic (pink) cytoplasm due to the dissolution of the Nissl bodies (central chromatolysis) characterize degenerating neurons. Increased cellularity is due to the proliferation of satellite cells (satellitosis) and inflammatory cell infiltrates. Some mononuclear cells infiltrate and phagocytose the neuronal cell body (neuronophagia). Lower right picture shows immunostaining for the transgene encoded by AAV (GFP in this case). The neurons displaying degenerative changes and mononuclear cell infiltrates are the ones that show the strongest protein expression (evidenced by dark brown staining on IHC). (FIG. 1C) Examples of grade 1 to grade 5 DRG lesion and grade 1 to grade 4 dorsal spinal cord axonopathy. Severity grades are defined as follows: 1 minimal (<10%), 2 mild (10-25%), 3 moderate (25-50%), 4 marked (50-95%), and 5 severe (>95%). Grade 5 was never observed in spinal cord. Arrows and circles delineate neuronal degeneration with mononuclear cell infiltrates in DRG (left column) and axonopathy (right column).

FIG. 2 shows an exemplary AAV expression cassette design for DRG-specific silencing. Four short tandem repeats of a miRNA reverse-complimentary sequence (miR targets or target sequences) are introduced between the stop codon and the poly-A. In DRG neurons, miR-183 binds the 3′ untranslated region of the mRNA and recruits the RNA-induced silencing complex (RISC), which in turn leads to silencing through mRNA cleavage. In other cell types that do not express miR-183, translation and protein synthesis occur without any impact from the 3′ UTR region.

FIGS. 3A -3D shows miR-183 target sequences specifically silence transgene expression in vitro and in mice DRG neurons. (FIG. 3A) We transiently co-transfected 293 cells with GFP expressing AAV plasmids harboring miR-183 or miR-145 targets, and control or miR-183-expression vector. We detected GFP protein levels 72 hrs post-transfection and quantified the levels with Western blotting. Experiments were performed in triplicates. Error bars indicate standard deviation. (FIG. 3B) We injected C57BL6/J mice IV with AAV9.CB7.GFP control vector or AAV9.CB7.GFP-miR vectors at the dose of 4 x 10¹² gc. We screened three DRG-enriched miRs: miR-183, miR-145, and miR-182. We harvested DRG two weeks post-injection and stained for GFP using IHC. Using the ImageJ cell-counter tool, we counted the percentage of GFP-expressing neurons over total DRG neurons. Wilcoxon test, * p<0.05, ** p<0.01, *** p<0.001. (FIG. 3C) Here we show representative pictures of GFP immunostainings from DRG quantified in panel FIG. 3B. (FIG. 3D) We injected C57BL6/J mice IV with AAV-PHP.B.CB7.GFP control vector or AAV-PHP.B.CB7.GFP-miR (miR-183, miR-145, miR-182). We harvested CNS and liver three weeks post-injection for direct GFP observation using fluorescent microscopy. Here we show representative pictures of cerebellum, cortex, and liver.

FIGS. 4A - 4C show miR-183 targets specifically silence GFP expression in DRG and decrease toxicity after AAVhu68.GFP ICM administration to NHP. We injected adult rhesus macaques ICM with 3.5 x 10¹³ GC of AAVhu68.CB7.GFP control vector (n=2; 1 male, 1 female, 5 and 8 years old, respectively) or AAVhu68.CB7.GFP-miR-183 (n=4, 4 female, 5-6 years old). Half of the animals were sacrificed two weeks post-injection for GFP expression analysis and the other half were sacrificed two months post-injection for GFP expression and histopathology. (FIG. 4A) Representative pictures of GFP-immunostained sections of DRG, spinal cord motor neurons, cerebellum, cortex, heart, and liver two weeks post-vector administration. (FIG. 4B) Quantification of GFP-positive cells in DRG (sensory neurons), spinal cord (lower motor neurons), cerebellum, and cortex in NHP (n=2 AAV.GFP, n=4 AAV.GFP-miR-183). For DRG, a minimum of two whole lumbar DRG sections per animal were quantified representing at least 300 neurons per animal. Each data point represents one distinct section. For cerebellum and cortex, a minimum of five 20x magnification fields were quantified per region and per animal using the ImageJ cell-counter tool. Data shown as mean; error bars indicate standard deviation. Wilcoxon test, * p<0.05, ** p<0.01, *** p<0.001. (FIG. 4C) Histopathology two months after injection shows severity grades of dorsal spinal cord axonopathy, peripheral nerves axonopathy (median, peroneal and radial nerves), and DRG neuronal degeneration and mononuclear infiltration. A board-certified Veterinary Pathologist who was blinded to the vector group established severity grades, which were defined as follows: 1 minimal (<10%), 2 mild (10-25%), 3 moderate (25-50%), 4 marked (50-95%) and 5 severe (>95% - not observed). Each bar represents one animal. 0 represents absence of lesion.

FIG. 5 shows miR-183 targets specifically silence hIDUA expression in DRG after AAVhu68.hIDUA ICM administration to NHP. We injected adult rhesus macaques ICM with either 1) 1 x 10¹³ GC of AAVhu68.CB7.hIDUA control vector (n=3, 2 female, 1 male, age 2.5 years old); 2) AAVhu68.CB7.hIDUA control vector with prophylactic steroids treatment (1 mg/kg/day of prednisolone from day minus 7 to day 30 followed by progressive taper off, n=3, 3 male, age 2.5-3.5 years old); or 3) AA Vhu68.CB7.hIDUA-miR--183 (containing miR-183 targets) (n=3, 2 male, 1 female, age 2.25-5 years old). Animals were sacrificed three months post-injection to analyze transgene expression and histopathology. Representative pictures show the analysis of hIDUA expression by anti-hIDUA antibody immunofluorescence (DRG, first row), anti-hIDUA IHC (lower motor neurons, cerebellum, cortex), and anti-IDUA ISH (DRG last row). hIDUA ISH: exposure time is 200 ms for AAVhu68.hIDUA with and without steroids. Sensory neurons show massive transgene mRNA expression. Exposure time is 1 s for AAV.hIDUA-miR-183. Sensory neurons have low ISH signal (mRNA) in the nucleus and cytoplasm. mRNA is visible in satellite cells that surround neurons at this higher exposure time.

FIGS. 6A - 6C shows miR-183 mediated silencing is specific to DRG neurons and fully prevents DRG toxicity in NHP treated ICM with AAVhu68.hIDUA. (FIG. 6A) Quantification of hIDUA-positive cells in DRG (sensory neurons), spinal cord (lower motor neurons), cerebellum, and cortex in NHP (n=3 per group). A minimal of five 20x magnification fields per region were quantified per animal. Error bars represent standard deviation. Wilcoxon test, * p<0.05, ** p<0.01, *** p<0.001. (FIG. 6B) Histopathology scoring three months post-injection: dorsal axonopathy cumulative scores (sum of severity grades from cervical, thoracic, and lumbar segments - maximal possible score 15); DRG cumulative score (sum of severity grades from cervical, thoracic, and lumbar segments -maximal possible score 15) and median nerve score (sum of axonopathy and fibrosis severity grades - maximal possible score 10). A board-certified Veterinary Pathologist who was blinded to the vector group established severity grades defined as follows: 1 minimal (<10%), 2 mild (10-25%), 3 moderate (25-50%), 4 marked (50-95%) and 5 severe (>95% -not observed). 0 represents absence of lesion. Error bars represent standard deviation. (FIG. 6C) ISH using hIDUA transgene-specific probes, high magnification of DRG sensory neurons and satellite cells; 1 s exposure time with blue DAPI nuclear counterstain. Arrows: DRG sensory neurons; arrowheads: satellite cells.

FIGS. 7A - 7D show T cell and antibody responses to hIDUA in NHP. Adult rhesus macaques were injected ICM with either 1) 1 × 10¹³ GC of AAVhu68.CB7.hIDUA control vector (n=3); 2) AAVhu68.CB7.hIDUA control vector with prophylactic steroids treatment (1 mg/kg/day of prednisolone from day minus 7 to day 30 followed by progressive taper off, n=3); or 3) AAVhu68.CB7.hIDUA-miR-183 (n=3). (FIGS. 7A - 7C) Interferon gamma ELISPOT responses in lymphocytes isolated from PBMC, spleen, liver, and deep cervical lymph nodes 90 days post injection. Each animal has three values representing a different peptide pool (three overlapping peptide pools to cover the entire hIDUA sequence). Red indicates a positive ELISPOT response defined as >55 spot-forming units per 106 lymphocytes and three times the medium negative control upon no stimulation. (FIG. 7D) anti-hIDUA antibody ELISA assay, serum dilution 1: 1,000.

FIG. 8 shows concentration of cytokines/chemokines in the CSF. Samples were collected at time of vector administration (D0) and 24 hours (24h), 21 (D21) and 35 (D35) days after vector administration. Heat maps showing the concentration from a Milliplex MAP kit containing the following analytes: sCD137, Eotaxin, sFasL, FGF-2, Fractalkine, Granzyme A, Granzyme B, IL-1α, IL-2, IL-4, IL-6, IL-16, IL-17A, IL-17E/IL-25, IL-21, IL-22, IL-23, IL-28A, IL-31, IL-33, IP-10, MIP-3α, Perform, and TNFβ.

FIG. 9 shows vector biodistribution in brain, spinal cord, and DRG in NHP. Adult rhesus macaques were injected ICM with either 1) 1 × 10¹³ GC of AAVhu68.CB7.hIDUA control vector (n=3); 2) AAVhu68.CB7.hIDUA control vector with prophylactic steroids treatment (1 mg/kg/day of prednisolone from day minus 7 to day 30 followed by progressive taper off, n=3); or 3) AAVhu68.CB7.hIDUA-miR-183 (n=3). NHP tissue DNA was extracted with a QIAamp DNA Mini Kit. Vector genomes were quantified by real-time polymerase chain reaction using Taqman reagents and primers/probes that target the rBG polyadenylation sequence of the vectors. Results are expressed in genome copy per diploid genome. Error bars represent standard deviation.

FIG. 10A and FIG. 10B show the results of a study on sponge effect involving an analysis of miR-183 cluster-regulated gene expression in NHPs following delivery of AAV-IDUA or AAV-IDUA-4XmiR-183. FIG. 10A provides a miR-183 cluster regulated gene mRNA quantification in dorsal root ganglia (DRG). FIG. 10B provides the results from analysis of the cortex. There is no increased expression of miR-183 cluster-regulated genes (CACNA2D1 or CACNA2D2), comparing AAV-IDUA or AAV-IDUA-miR-183 treated animals in either DRG (high miR-183 abundance) or frontal cortex (low miR-183 abundance).

FIG. 11 shows results of transduction with AAV9 vectors carrying an eGFP transgene with or without four copies of the miR-183 detargeting sequences at low (5 x10⁵) or high (2.5 x 10⁸) dose. The low and high dose without miR-183 was tested with or without adenovirus type 5 (Ad5) helper co-transfection at a multiplicity of infection (MOI) of 100 (for low dose AAV9-eGFP) or 10 (high dose AAV9-eGFP). All DRG neurons were transduced, and no visible signs of toxicity were observed. No GFP expression was observed in DRG neurons, while some expression was observed in fibroblast-like cells. The findings confirmed repression of GFP transcription with the 4x-miR-183 target expression cassettes.

FIG. 12 shows results from a “sponge effect” study in rat DRG cells. These data show that miR-183 levels in rat DRG cells are decreased when cells are transduced with AAV9-eGFP-miR-183 vectors. AAV9-eGFP-miR-183 showed target engagement on the GFP-miR-183 mRNA.

FIGS. 13A - 13C show results from a “sponge effect” study in rat DRG cells where expression of three known miR-183 regulated transcripts was determined. FIG. 13A shows relative expression of CACANA2D1in rat DRG cells following administration of AAV-GFP or AAV-GFP-miR-183 vectors (or mock vector control). FIG. 13B shows relative expression of CACANA2D2 in rat DRG cells following administration of AAV-GFP or AAV-GFP-miR-183 vectors (or mock vector control). FIG. 13C shows relative expression of ATF3 in rat DRG cells following administration of AAV-GFP or AAV-GFP-miR-183 vectors (or mock vector control). No changes in the mRNA levels of these three miR-183-regulated transcripts were observed.

FIG. 14 shows neuroanatomy and microscopic findings. Neuronal cell bodies of the DRG (A) project axons centrally into the ascending (sensory) dorsal white matter tracts of the spinal cord (C) and into the peripheral nervous system (D). (A1-D1) Neuroanatomical relationship of the microscopic lesions associated with DRG pathology. Neuronal cell body degeneration (circles, A1) in the DRG results in axonal degeneration (vertical arrows, B1) with or without periaxonal fibrosis (horizontal arrows, B1) extending both centrally and peripherally in the nerve root. Axonal degeneration in the DRG nerve root extends centrally into the ascending dorsal white matter tracts of the spinal cord (vertical arrows, C1) and into peripheral nerves (vertical arrows, D1) with or without periaxonal fibrosis (horizontal arrows, D1). (A2-D2) Normal DRG, DRG nerve root, dorsal white matter of spinal cord and peripheral nerve. (Hematoxylin and eosin; 20x, Scale bar = 100 µm). (E-H) High magnification images of varying stages of DRG pathology. (E) Early in the degenerative process, the neuronal cell bodies appear relatively normal (circles) with only proliferating satellite cells along with microglial cells and infiltrating mononuclear cells (neuronophagia). (F) As the lesions progress, the neuronal cell bodies exhibit evidence of degeneration (vertical arrow) characterized by small, irregular- or angular-shaped cells with fading or loss of nuclei and cytoplasmic hypereosinophilia. (G) Neuronal cell body degeneration (circles) can result in complete obliteration (star) by satellite cells, microglial cells and mononuclear cells; this is considered end-stage degeneration. (H) Normal DRG. (Hematoxylin and eosin; 40x, Scale bar = 50 µm)

FIGS. 15A - 15D show effects of study characteristics on severity of DRG pathology. Average pathology scores in DRG (black) and dorsal spinal cord (SC) axons (grey) with different (FIG. 15A) routes of administration, (FIG. 15B) vector doses, (FIG. 15C) times post-injection for tissue collection, and (FIG. 15D) study conduct compliance with GLP guidelines. Mean results with standard error of mean; tables indicate number of animals (n) and number of histological sections scored (count) in each group. The comparison between groups was done using Wilcoxon rank-sum test within each DRG and spinal cord regions (i.e., cervical, thoracic, lumbar) and the combined p-value was calculated for the overall DRG or spinal cord inter-group comparison using Fisher’s method with statistical significance assessed at the 0.05 level. * indicate significance for inter-group comparison and # indicate (FIG. 15A) significance for comparison with the vehicle control group, or (FIG. 15C) significance for comparison with the 180+ day time point. *, # p<0.05; **, ## p<0.01; ***, ### p<0.001; ****, #### p<0.0001. Color code for statistics symbols: black for DRG and grey for SC.

FIG. 16A and FIG. 16B show effects of animal characteristics on severity of DRG pathology. Average pathology scores in DRG (black) and dorsal spinal cord (SC) axons (grey) with different (FIG. 16A) age of the animals at injection, and (FIG. 16B) sex of the animals (rhesus macaques only). Mean results with standard error of mean; tables indicate number of animals (n) and number of histological sections scored (count) in each group. The comparison between groups was done using Wilcoxon rank-sum test within each DRG and spinal cord regions (i.e., cervical, thoracic, lumbar) and the combined p-value was calculated for the overall DRG or spinal cord inter-group comparison using Fisher’s method with statistical significance assessed at the 0.05 level. For FIG. 16A, * indicates significance for inter-group comparison and # indicates significance for comparison with the infant age group. *, # p<0.05; **, ## p<0.01; ***, ### p<0.001; ****, #### p<0.0001. Color code for statistics symbols: black for DRG and grey for SC.

FIGS. 17A - 17D show effects of vector characteristics on severity of DRG pathology. Average pathology scores in DRG (black) and dorsal spinal cord (SC) axons (grey) with different (FIG. 17A) capsids, (FIG. 17B) promoters, and (FIG. 17C) transgenes, and secreted vs. non-secreted transgenes (FIG. 17D). Transgenes were arranged from 1 to 20 based on the severity of SC pathology. Mean results with standard error of mean; tables indicate number of animals (n) and number of histological sections scored (count) in each group. (FIG. 17A, FIG. 17B, and FIG. 17D). The comparison between groups was done using Wilcoxon rank-sum test within each DRG and spinal cord regions (i.e., cervical, thoracic, lumbar) and the combined p-value was calculated for the overall DRG or spinal cord inter-group comparison using Fisher’s method with statistical significance assessed at the 0.05 level. * indicate significance for inter-group comparison: *p<0.05; **p<0.01; ***p<0.001; ****p<0.0001. Color code for statistics symbols: black for DRG and grey for SC. No statistical analysis was done for the transgene comparison due to small n for some groups.

FIG. 18 shows regional pathology scores with distribution of severity grades. Mean percentage proportion of pathology scores with standard error of mean (red dots and bars) and distribution of severity grades by region (stacked columns). Tables indicate number of animals (n) and number of histological sections scored (count) in each group. The comparison between means was done using Wilcoxon rank-sum test between TRG and DRG and between DRG and SC respective regions (i.e., cervical, thoracic, and lumbar). Statistical significance was assessed at the 0.05 level. * indicate significance for trigeminal nerve ganglion (TRG) to DRG comparisons; # indicate significance for DRG to SC regional comparisons. ** p<0.01; #### p<0.0001.

FIG. 19A and FIG. 19B show peripheral nerve pathology. Mean percentage proportion of pathology scores with standard error of mean (red dots and bars), and distribution of severity grades by peripheral nerve (stacked columns). Tables indicate number of animals (n) and number of histological sections scored (count) in each group. No statistical analysis was performed as some peripheral nerves were not collected in a majority of studies.

FIGS. 20A - 20D show effects of study characteristics on severity of DRG pathology split by spinal region. Average pathology scores in DRG (black) and dorsal spinal cord (SC) axons (grey) regions with different (FIG. 20A) routes of administration, (FIG. 20B) vector doses, (FIG. 20C) times post-injection for tissue collection, and (FIG. 20D) study conduct compliance with GLP guidelines. Mean results with standard error of mean; tables indicate number of animals (n) and number of histological sections scored (count) in each group. C = cervical, T = thoracic, L = lumbar regions.

FIG. 21A and FIG. 21B show effects of animal characteristics on severity of DRG pathology split by spinal region. Average pathology scores in DRG (black) and dorsal spinal cord (SC) axons (grey) regions with different (FIG. 21A) age of the animals at injection, and (FIG. 21B) sex of the animals (rhesus macaques only). Mean results with standard error of mean; tables indicate number of animals (n) and number of histological sections scored (count) in each group. C = cervical, T = thoracic, L = lumbar regions.

FIGS. 22A - 22C show effects of vector characteristics on severity of DRG pathology split by spinal region. Average pathology scores in DRG (black) and dorsal spinal cord (SC) axons (grey) regions with different (FIG. 22A) capsids, (FIG. 22B) promoters, and (FIG. 22C) transgenes. Transgenes were arranged from 1 to 20 based on the severity of SC pathology. Mean results with standard error of mean; tables indicate number of animals (n) and number of histological sections scored (count) in each group. C = cervical, T = thoracic, L = lumbar regions.

FIG. 23 shows the effect of secreted vs. non-secreted transgene on severity of DRG pathology by spinal region. Average pathology scores in DRG (black) and dorsal spinal cord axons (grey) regions with secreted or non-secreted transgenes. Mean results with standard error of mean; tables indicate number of animals (n) and number of histological sections scored (count) in each group. C = cervical, T = thoracic, L = lumbar regions.

FIG. 24 shows GFP expression in brain cortex. C57BL6/J mice were injected IV with a AAV-PHP.B.GFP control vector or a AAV-PHP.B.GFP-miR target vector at a dose of 1 x 10¹² GC, n=4 per group.

FIGS. 25A - 25C show GFP expression following administration of AAV9.GFP vectors having miR-183, miR-182, or miR-145 target sequences. C57BL6/J mice were injected IV with 4x10¹² GC of vector encoding GFP with tandem repeats of miR-183 targets (4X repeats) (AAV9. CB7.CI.eGFP.miR-183.rBG), miR-182 targets (4X repeats) (AAV9. CB7.CI.eGFP.miR-145.rBG), miR-145 targets (4X repeats) (AAV9. CB7.CI.eGFP.miR-182.rBG), or a no miR target control (AAV9.CB7.CI.eGFP.rBG). n=3-4 per group. The vector modified with miR-145 targets showed decreased GFP expression in heart tissue compared to the control vector with no miR target sequences. The vector modified with 4x miR-183 target sequences showed increased GFP expression in heart tissue compared to the vector with no miR targets and the miR-145 target vectors. The vector with miR-183 target sequences showed increased GFP expression in brain cortex and brainstem compared to the vector with miR-145 target sequences and the vector with no miR target sequences.

FIG. 25D shows quantification of GFP direct fluorescence intensity from the results shown in FIGS. 25A - 25C. 1-way ANOVA followed by Tukey’s multiple comparison test. * p<0.05, ** p<0.01.

FIG. 26A shows expression of miR-96, miR-182, and miR-183 in HCT116 cells. Expression is shown relative to miR-96.

FIG. 26B shows expression of miR-182 and miR-183 in HCT116 cells relative to expression levels in Neuro2a(N2A) cells.

FIG. 26C shows relative expression levels of miR-96, miR-182, and miR-183 in HCT116, rat DRG, rhesus(RH)-DRG, and human(HU)-DRG cells. Expression levels are shown relative to miR-96 in HCT116 cells.

FIGS. 27A - 27D show evaluation of GFP expression in HCT116 cells following transduction with AA9.GFP vectors have an increasing number (1x-8x) of miR-183 target sequences (AA VCB7.CI.eGFP.miR-182(1x-8x).rBG), 4x miR-182 target sequences (AAV.CB7.CI.eGFP.miR-182(4x).rBG), or 4x miR-182 target sequences + 4x miR-183 target sequences (AAV.CB7.CI.eGFP.miR-182(4x).miR-183(4x).rBG). FIG. 27A shows fluorescence microscopy and FIG. 27B shows flow cytometric analysis of transduced cells. FIG. 27C and FIG. 27D show quantification of results from flow cytometric analysis, as provided in FIG. 27B.

FIGS. 28A - 28J show results from a mouse study to evaluate the effects of miR target sequences on transgene expression. AAVhu68.GFP (no miR target sequences), AAVhu68.GFP-miR-183(4x), AA Vhu68. GFP-miR-182(4x), and AAVhu68.GFP-miR-182-miR-183(4x+4x) vectors were administered IV (4 x 10¹² GC) or ICV (1 x 10¹¹ GC). Mice were sacrificed four weeks post-administration. (FIG. 28A and FIG. 28B) IHC for transgene (GFP) expression in DRG and quantification of findings. (FIGS. 28C - 28E) IHC for transgene (GFP) expression in brain and spinal cord and quantification of findings. (FIGS. 28F - FIG. 28J) IHC for transgene (GFP) expression in liver, kidney, heart, and quadriceps muscle and quantification of findings.

FIGS. 29A - 29D show results from an NHP study to evaluate the effects of miR target sequences of transgene expression. AAVhu68.GFP (no miR target sequences), AAVhu68.GFP-miR-182(4x), and AAVhu68.GFP-miR-182-miR-183(4x+4x) vectors were administered ICM (3 x 10¹³ GC). Animals were sacrificed five weeks post-administration. (FIG. 29A) IHC for transgene (GFP) expression in DRG (FIG. 29A) and spinal cord (FIG. 29B) from cervical, thoracic, and lumbar regions. (FIG. 29C and FIG. 29D) Scoring of DRG toxicity / secondary axonopathy. Vectors with miR target sequences demonstrated similar silencing of GFP expression and reduction of pathology.

FIGS. 30A - 30C show the incidence and severity of background DRG/TRG (FIG. 30A), spinal cord (FIG. 30B), and peripheral nerve (FIG. 30C) findings in control animals (naive and ICM vehicle-administered) across multiple studies.

FIG. 31A and FIG. 31B show the incidence and severity of background DRG toxicity in historical control animals (naive and ICM vehicle-administered) across multiple studies.

### DETAILED DESCRIPTION OF THE INVENTION

The compositions and methods provided herein are useful in therapies for gene delivery for repressing transgene expression in DRG neurons through the use of miRNA target sequences. As used herein, the term “repression” includes partial reduction or complete extinction or silencing of transgene expression. Transgene expression may be assessed using an assay suitable for the selected transgene. The compositions and methods provided decrease toxicity of the DRG characterized by neuronal degeneration, secondary dorsal spinal cord axonal degeneration, and/or mononuclear cell infiltrate. In certain embodiments, the expression cassette or vector genome comprises miRNA target sequences in the untranslated region (UTR) 3′ to a gene product coding sequence. As provided herein, the expression cassette or vector genome comprises at least eight miR target sequences. In certain embodiments, each target sequence is independently selected and is specific for miR-183 or miR-182. In certain embodiments, an expression cassette comprises four independently selected miR-183 target sequences and four independently selected miR-182 target sequences, wherein the miR target sequences are operably linked to the 3′ end of the coding sequence. In other embodiments, an expression cassette comprises eight miR-183 target sequences or eight miR-183 target sequences. Other combinations of miR sequences may be selected as described herein. Suitably, two or more miRNA target sequences are provided in tandem, optionally separated by a spacer sequence. In certain embodiments, three or more miRNA target sequences are provided in tandem, optionally separated by a spacer sequence. In certain embodiments, eight miRNA sequences are provided in tandem, optionally separated by spacer sequences. A variety of delivery systems may be used to deliver the expression cassette to a subject, e.g., a human patient. Such delivery systems may be a viral vector, a non-viral vector, or a non-vector-based system (e.g., a liposome, naked DNA, naked RNA, etc.). These delivery systems may be used for delivery directly to the central nervous system (CNS), peripheral nervous system (PNS), or for intravenous or an alternative route of delivery. In other embodiments, these compositions and methods are used for systemic delivery of gene therapy vectors (e.g., rAAV). In certain embodiments, these compositions and methods are useful where high doses of vector (e.g., rAAV) are delivered. In certain embodiments, the compositions and methods provided herein permit a reduced dose, reduced length, and/or reduced number of immunomodulators to be co-administered with a gene therapy vector (e.g., a rAAV-mediated gene therapy). In certain embodiments, the compositions and methods provided herein eliminate the need to co-administer immunosuppressants or immunomodulatory therapy prior to, with, and/or following administration of a viral vector (e.g. a rAAV).

A “5′ UTR” is upstream of the initiation codon for a gene product coding sequence. The 5′ UTR is generally shorter than the 3′ UTR. Generally, the 5′ UTR is about 3 nucleotides to about 200 nucleotides in length, but may optionally be longer.

A “3′ UTR” is downstream of the coding sequence for a gene product and is generally longer than the 5′ UTR. In certain embodiments, the 3′ UTR is about 200 nucleotides to about 800 nucleotides in length, but may optionally be longer or shorter.

As used herein, an “miRNA” or “miR” refers to a microRNA which is a small non-coding RNA molecule that regulates mRNA and reduces its translation to protein. The miRNA contains a “seed sequence” which is a region of nucleotides which specifically binds to mRNA by complementary base pairing, leading to destruction or silencing of the mRNA. In certain embodiments, the seed sequence is located on the mature miRNA (5′ to 3′) and is generally located at position 2 to 7 or 2 to 8 (from the 5′ end of the sense (+) strand) of the miRNA, although it may be longer than in length. In certain embodiments, the length of the seed sequence is no less than about 30% of the length of the miRNA sequence, which may be at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides.

As used herein, an “miRNA target sequence” or “miR target sequence” is a sequence located on the DNA positive strand (5′ to 3′) and is at least partially complementary to a miRNA sequence, including the miRNA seed sequence. The miRNA target sequence is exogenous to the untranslated region of the encoded transgene product and is designed to be specifically targeted by miRNA in cells in which repression of transgene expression is desired. The term “miR-183 cluster target sequence” refers to a target sequence that responds to one or members of the miR-183 cluster (alternatively termed family), including miR-183, -96 and -182 (as described by Dambal, S. et al. Nucleic Acids Res 43:7173-7188, 2015, which is incorporated herein by reference).Without wishing to be bound by theory, the messenger RNA (mRNA) for the transgene (encoding the gene product) is present in a cell type to which the expression cassette containing the miRNA is delivered, such that specific binding of the miRNA to the 3′ UTR miRNA target sequences results in mRNA silencing and cleavage, thereby reducing or eliminating transgene expression only in the cells that express the miRNA.

Typically, the miRNA target sequence is at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides, and which contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two or three copies) of the sequence which is 100% complementary to the seed sequence. In certain embodiments, the region of 100% complementarity comprises at least 30% of the length of the target sequence. In certain embodiments, the remainder of the target sequence has at least about 80 % to about 99% complementarity to the miRNA. In certain embodiments, in an expression cassette containing a DNA positive strand, the miRNA target sequence is the reverse complement of the miRNA.

In certain embodiments, provided herein are engineered expression cassettes or vector genomes comprising at least one copy of an miR target sequence directed to one or more members of the miR-183 family or cluster operably linked to a transgene to repress expression of the transgene in DRG and/or reduce or eliminate DRG toxicity and/or axonopathy. In certain embodiments, the engineered expression cassette or vector genome comprises multiple miRNA target sequences, such that the number of miRNA target sequences is sufficient to reduce or minimize transgene expression in DRG to reduce and/or eliminate DRG toxicity and/or axonopathy. The expression cassette or vector genome may be delivered via any suitable carrier system, viral vector or non-viral vector, via any route, but is particularly useful for intrathecal administration.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration directly into the cerebrospinal fluid of the cisterna magna cerebellomedularis, more specifically via a suboccipital puncture or by direct injection into the cisterna magna or via permanently positioned tube.

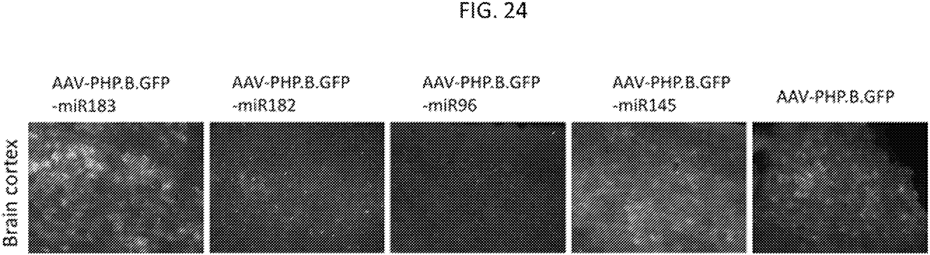

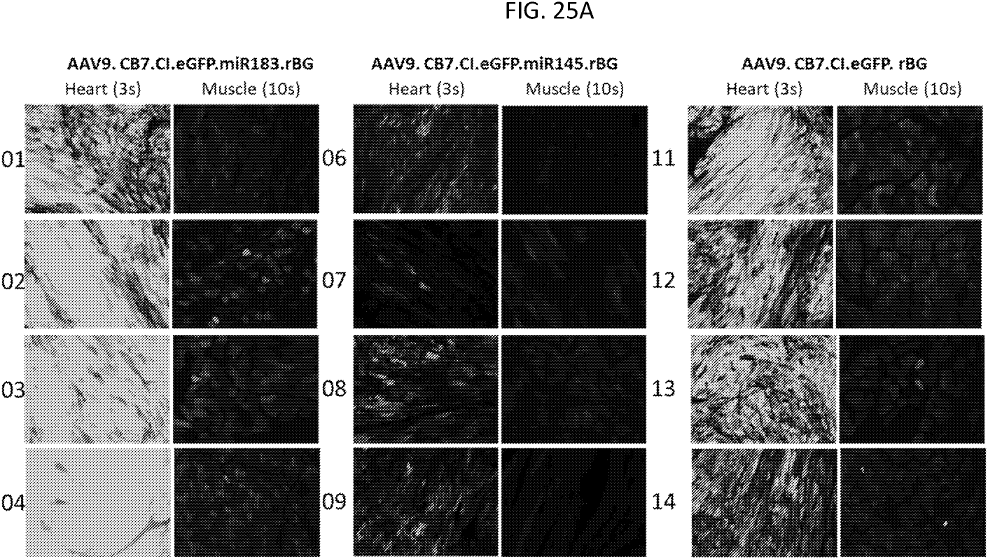

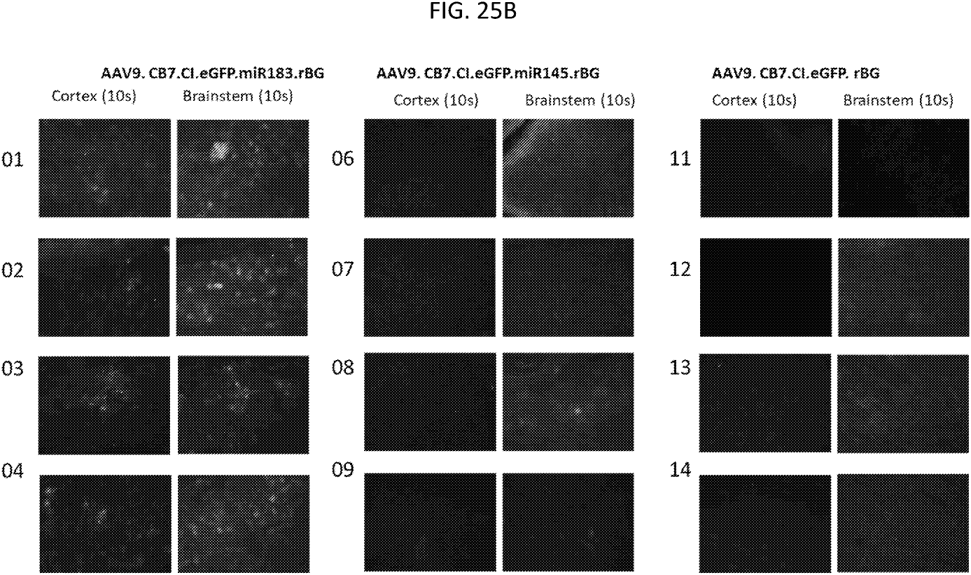

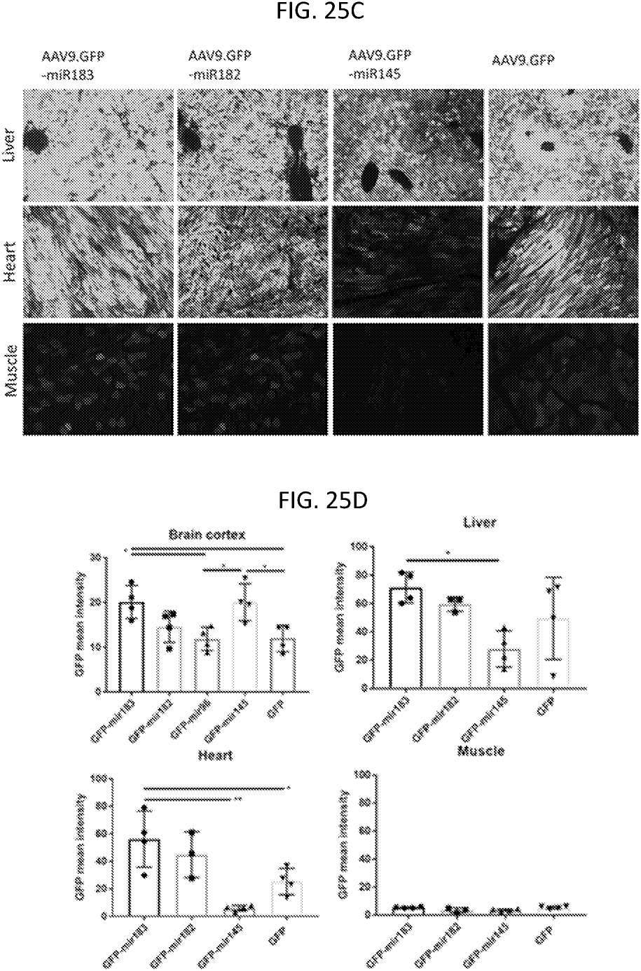

Unexpectedly, compositions comprising the miR-183 target sequences described herein for repressing expression in the DRG have been observed to provide enhanced transgene expression in one or more different cell types (other than the DRG) within the central nervous system, including, but not limited to, neurons (including, e.g., pyramidal, purkinje, granule, spindle, and interneuron cells) or glial cells (including, e.g., astrocytes, oligodendrocytes, microglia, and ependymal cells). While this observation was initially made following an intrathecal delivery route, this expression -enhancing effect is not limited to CNS-delivery routes. Enhanced expression has also been observed following intravenous delivery and may also be achieved using other routes, e.g., intravenous (e.g., particularly high dose delivery), intramuscular (particularly high dose delivery), or other systemic delivery routes. In certain embodiments, compositions comprising the miR-183 target sequences described herein provide enhanced transgene expression in heart tissue (see FIG. 24A). For example, the inventors have observed a statistically significant reduction of GFP expression in DRG with a mir-183-target containing vector compared with a control vector, whereas expression was enhanced in the lumbar motor neurons and cerebellum. This enhanced expression was also associated with a remarkable reduction of pathology across the DRG and eight other regions, i.e., dorsal spinal axonopathy at cervical, thoracic, and lumbar spine, and axonopathy of median, peroneal, and radial nerves.

In certain embodiments, one may wish to select miR-182 target sequences and/or miR-96 target sequences for expression cassettes comprising transgenes which are not targeted to the CNS, so as to avoid enhancing CNS expression of the transgene (while repressing DRG expression). For example, expression cassettes comprising transgenes for delivery to skeletal muscle or the liver may wish to avoid any enhancement of CNS expression, but prevent DRG-toxicity and/or axonopathy which can be associated with the high doses which may be required.

In certain embodiments, the vector genome or expression cassette contains at least one miRNA target sequence that is a miR-183 target sequence. In certain embodiments, the vector genome or expression cassette contains an miR-183 target sequence that includes AGTGAATTCTACCAGTGCCATA (SEQ ID NO:1), where the sequence complementary to the miR-183 seed sequence is underlined. In certain embodiments, the vector genome or expression cassette contains more than one copy (e.g. two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 1 and, thus, when aligned to SEQ ID NO: 1, there are one or more mismatches. In certain embodiments, a miR-183 target sequence comprises a sequence having at least 1, 2, 3, 4, 5, 6, 7, 8, 9, or 10 mismatches when aligned to SEQ ID NO: 1, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 1, i.e., a sequence that lacks at least 1, 2, 3, 4, 5, 6, 7, 8, 9, or 10 nucleotides at either or both the 5′ or 3′ ends of SEQ ID NO: 1. The expression cassette or vector genome comprises at least eight miR target sequences. In certain embodiments, each target sequence is independently selected and is specific for miR-183 or miR-182. In certain embodiments, an expression cassette comprises 4 independently selected miR-183 target sequences and 4 independently selected miR-182 target sequences, wherein the miR target sequences are operably linked to the 3′ end of the coding sequence. In other embodiments, an expression cassette comprises 8 miR-183 target sequences or 8 miR-183 target sequences. Other combinations of miR sequences may be selected as described herein. In certain embodiments, the expression cassette or vector genome comprises a transgene and one miR-183 target sequence. In yet other embodiments, the expression cassette or vector genome comprises at least two, at least three,at least four, at least five, at least six, or at least seven miR-183 or miR-182 target sequences. In yet other embodiments, the expression cassette or vector genome comprises eight miR-183 target sequences.

Compositions comprising a transgene and miR-182 have been observed to minimize or eliminate dorsal root ganglia toxicity and/or prevent axonopathy. However, while effective for this purpose, the expression cassettes or vector genomes containing miR-182 target sequence have not been observed to enhance CNS expression as was unexpectedly found in the composited which had the miR-183 target sequence. Thus, these compositions may be desirable for genes to be targeted outside the CNS. In certain embodiments, provided herein is an expression cassette or vector genome that comprises one or more miR-183 family target sequences and lacks a transgene (i.e. the miR-183 family target sequence(s) is not operably linked to a sequence encoding a heterologous gene product).

As provided herein, the expression cassette or vector genome comprises at least eight miR target sequences. In certain embodiments, each target sequence is independently selected and is specific for miR-183 or miR-182. In certain embodiments, an expression cassette comprises four independently selected miR-183 target sequences and four independently selected miR-182 target sequences, wherein the miR target sequences are operably linked to the 3′ end of the coding sequence. In other embodiments, an expression cassette comprises eight miR-183 target sequences or eight miR-183 target sequences. Other combinations of miR sequences may be selected as described herein. In certain embodiments, the vector genome or expression cassette contains at least one miRNA target sequence that is a miR-182 target sequence. In certain embodiments, the vector genome or expression cassette contains an miR-182 target sequence that includes AGTGTGAGTTCTACCATTGCCAAA (SEQ ID NO: 3). In certain embodiments, the vector genome or expression cassette contains more than one copy (e.g. two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 3 and, thus, when aligned to SEQ ID NO: 3, there are one or more mismatches. In certain embodiments, a miR-183 target sequence comprises a sequence having at least 1, 2, 3, 4, 5, 6, 7, 8, 9, or 10 mismatches when aligned to SEQ ID NO: 3, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 3, i.e., a sequence that lacks at least 1, 2, 3, 4, 5, 6, 7, 8, 9, or 10 nucleotides at either or both the 5′ or 3′ ends of SEQ ID NO: 3. In certain embodiments, the expression cassette or vector genome comprises a transgene and one miR-182 target sequence. In yet other embodiments, the expression cassette or vector genome comprises at least two, three or four miR-182 target sequences.

In certain embodiments, an expression cassette or vector genome has two or more consecutive miRNA target sequences are continuous and not separated by a spacer. In certain embodiments, wherein two or more of the miRNA target sequences are separated by a spacer. In certain embodiments, the spacer is a non-coding sequence of about 1 to about 12 nucleotides, or about 2 to about 10 nucleotides in length, or about 3 to about 10 nucleotides, about 4 to about 6 nucleotide in length, or 3, 4, 5, 6, 7, 8, 9, 10 or 11 nucleotide in length.. Optionally, a single expression cassette may contain three or more miRNA target sequences, optionally having different spacer sequences therebetween. In certain embodiments, one or more spacer is independently selected from (i) GGAT (SEQ ID NO:5); (ii) CACGTG (SEQ ID NO: 6); or (iii) GCATGC (SEQ ID NO: 7). In certain embodiments, a spacer is located 3′ to the first miRNA target sequence and/or 5′ to the last miRNA target sequence. In certain embodiments, the spacers between the miRNA target sequences are the same.

In certain embodiments, an expression cassette comprises a transgene and one miR-183 target sequence and one or more different miRNA target sequences. In certain embodiments, expression cassettes contains miR-96 target sequence: mRNA and on DNA positive strand (5′ to 3′): AGCAAAAATGTGCTAGTGCCAAA (SEQ ID NO: 2); miR-182 target sequence: mRNA and on DNA positive strand (5′ to 3′): and/or AGTGTGAGTTCTACCATTGCCAAA (SEQ ID NO: 3).

Although miR-145 has been associated with brain in the literature, the studies to date have shown that miR-145 target sequences have no effect in reducing transgene expression in dorsal root ganglia. miR-145 target sequence: mRNA and on DNA positive strand (5′ to 3′): AGGGATTCCTGGGAAAACTGGAC (SEQ ID NO: 4).

As provided herein, expression cassettes and vector genomes contain transgenes operably linked, or under the control, of regulatory sequences which direct expression of the transgene product in the target cell. In certain embodiments, the expression cassette or vector genome contains a transgene that is operably linked to one or more miRNA target sequences provided herein. In certain embodiments, the expression cassette or vector genome is designed to contain multiple miRNA target sequences. The miRNA target sequences are incorporated into the UTR of the transgene (i.e., 3′ or downstream of the gene open reading frame).

The term “transgene” is used herein to refer to a DNA sequence from an exogenous source which is inserted into a target cell. The transgene is a nucleotide sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression of a gene produce in a target cell. The heterologous nucleic acid sequence (transgene) can be derived from any organism. An rAAV may comprise one or more transgenes. In certain embodiments, the transgene is gene editing enzyme (e.g. CRISPR-Cas enzyme or meganuclease). In further embodiments, transgene is a nucleotide sequence that is introduced (“knocked-in”) in a target cell genome. An expression cassette or vector genome may contain such a transgene alone or combination with a sequence encoding a gene editing enzyme.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain at least two, at least three, at least four, at least five, at least six, at least seven, or more of the same miRNA target sequence. In certain embodiments, the tandem repeats include up to eight miRNA target sequences which may be the same for different. In certain embodiment, the expression cassette contains eight miR-183 target sequence, e.g. seven identical target sequences separated by spacer sequences as provided in the vector genome of SEQ ID NO: 27 or eight identical target sequences separated by spacer sequences as provided in the vector genome of SEQ ID NO: 28. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

It is to be noted that the term “a” or “an”, refers to one or more, for example, “a vector”, is understood to represent one or more vector(s). As such, the terms “a” (or “an”), “one or more,” and “at least one” is used interchangeably herein.

As used herein, the term “about” means a variability of plus or minus 10 % from the reference given, unless otherwise specified.

### 1. Expression Cassette

An “expression cassette” as described herein, includes a nucleic acid sequence encoding a functional gene product operably linked to regulatory sequences which direct its expression in a target cell and miRNA target sequences in the UTR. As described herein, the miRNA target sequences are designed to be specifically recognized by miRNA present in cells in which transgene expression is undesirable and/or reduced levels of transgene expression are desired. In certain embodiments, the miRNA target sequences specifically reduce expression of the transgene in dorsal root ganglion. In certain embodiments, the miRNA target sequences are located in the 3′ UTR, 5′ UTR, and/or in both 3′ and 5′ UTR. The discussion of the miRNA target sequences found in this specification is incorporated by reference herein.

In one embodiment, the expression cassette is designed for expression in a human subject while reducing or eliminating DRG-expression of the transgene product. In one embodiment, the expression cassette is designed for expression in the central nervous system (CNS), including the cerebral spinal fluid and brain. In certain embodiments, the expression cassette or vector genome is designed for expression or enhanced expression of the transgene in one or more cell type present in the CNS (excluding the dorsal root ganglia), including nerve cells (such as, pyramidal, purkinje, granule, spindle, and interneuron cells) and glia cells (such as astrocytes, oligodendrocytes, microglia, and ependymal cells). In certain embodiments, enhanced expression of the transgene is achieved in one or more cell type with little to no expression of the transgene in another cell type of the CNS. In certain embodiments, the expression cassette is useful for expression in cells other than those of the CNS.

As used herein, the term “expression” or “gene expression” refers to the process by which information from a gene is used in the synthesis of a functional gene product. The gene product may be a protein, a peptide, or a nucleic acid polymer (such as a RNA, a DNA or a PNA).

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked.

As used herein, the term “operably linked” refers to both expression control sequences that are contiguous with the nucleic acid sequence encoding a gene product and/or expression control sequences that act in trans or at a distance to control the transcription and expression thereof.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g. a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

In one embodiment, the regulatory sequence comprises a promoter. In one embodiment, the promoter is a chicken β-actin promoter. In a further embodiment, the promoter is a hybrid of a cytomegalovirus immediate-early enhancer and the chicken β-actin promoter (a CB7 promoter). In another embodiment, a suitable promoter may include without limitation, an elongation factor 1 alpha (EF1 alpha) promoter (see, e.g., Kim DW et al, Use of the human elongation factor 1 alpha promoter as a versatile and efficient expression system. Gene. 1990 Jul 16;91(2):217-23), a Synapsin 1 promoter (see, e.g., Kügler S et al, Human synapsin 1 gene promoter confers highly neuron-specific long-term transgene expression from an adenoviral vector in the adult rat brain depending on the transduced area. Gene Ther. 2003 Feb;10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 Feb;145(2):613-9. Epub 2003 Oct 16), or a CB6 promoter (see, e.g., Large-Scale Production of Adeno-Associated Viral Vector Serotype-9 Carrying the Human Survival Motor Neuron Gene, Mol Biotechnol. 2016 Jan;58(1):30-6. doi: 10.1007/s12033-015-9899-5).

Suitable promoters may be selected, including but not limited to a constitutive promoter, a tissue-specific promoter or an inducible/regulatory promoter. Example of a constitutive promoter is chicken beta-actin promoter. A variety of chicken beta-actin promoters have been described alone, or in combination with various enhancer elements (e.g., CB7 is a chicken beta-actin promoter with cytomegalovirus enhancer elements; a CAG promoter, which includes the promoter, the first exon and first intron of chicken beta actin, and the splice acceptor of the rabbit beta-globin gene; a CBh promoter, SJ Gray et al, Hu Gene Ther, 2011 Sep; 22(9): 1143-1153). Examples of promoters that are tissue-specific are well known for liver (albumin, Miyatake et al., (1997) J. Virol., 71:5124-32; hepatitis B virus core promoter, Sandig et al., (1996) Gene Ther., 3: 1002-9; alpha-fetoprotein (AFP), Arbuthnot et al., (1996) Hum. Gene Ther., 7:1503-14), neuron (such as neuron-specific enolase (NSE) promoter, Andersen et al., (1993) Cell. Mol. Neurobiol., 13:503-15; neurofilament light-chain gene, Piccioli et al., (1991) Proc. Natl. Acad. Sci. USA, 88:5611-5; and the neuron-specific vgf gene, Piccioli et al., (1995) Neuron, 15:373-84), and other tissues. Alternatively, a regulatable promoter may be selected. See, e.g., WO 2011/126808B2, incorporated by reference herein.

In one embodiment, the regulatory sequence further comprises an enhancer. In one embodiment, the regulatory sequence comprises one enhancer. In another embodiment, the regulatory sequence contains two or more expression enhancers. These enhancers may be the same or may be different. For example, an enhancer may include an alpha mic/bik enhancer or a CMV enhancer. This enhancer may be present in two copies which are located adjacent to one another. Alternatively, the dual copies of the enhancer may be separated by one or more sequences.

In one embodiment, the regulatory sequence further comprises an intron. In a further embodiment, the intron is a chicken beta-actin intron. Other suitable introns include those known in the art may by a human β-globulin intron, and/or a commercially available Promega® intron, and those described in WO 2011/126808.

In one embodiment, the regulatory sequence further comprises a Polyadenylation signal (polyA). In a further embodiment, the polyA is a rabbit globin poly A. See, e.g., WO 2014/151341. Alternatively, another polyA, e.g., a human growth hormone (hGH) polyadenylation sequence, an SV40 polyA, or a synthetic polyA may be included in an expression cassette.

Expression cassettes can be delivered via any suitable non-viral vector delivery system or by a suitable viral vector. Suitable non-viral vector delivery systems are known in the art (see, e.g., Ramamoorth and Narvekar. J Clin Diagn Res. 2015 Jan; 9(1):GE01-GE06, which is incorporated herein by reference) and can be readily selected by one of skill in the art and may include, e.g., naked DNA, naked RNA, dendrimers, PLGA, polymethacrylate, an inorganic particle, a lipid particle, a polymer-based vector, or a chitosan-based formulation.

It should be understood that the description of the expression cassettes is intended to be applied to other compositions, regiments, aspects, embodiments and methods described across the Specification.

### 2. Vector

A “vector” as used herein is a biological or chemical moiety comprising a nucleic acid sequence which can be introduced into an appropriate target cell for replication or expression of said nucleic acid sequence. Examples of vectors include but are not limited to a recombinant virus, a plasmid, lipoplexes, a polymersome, polyplexes, a dendrimer, a cell penetrating peptide (CPP) conjugate, a magnetic particle, or a nanoparticle. In one embodiment, the vector is a nucleic acid molecule into which an exogenous or heterologous or engineered nucleic acid encoding a functional gene product, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In certain embodiments, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid - nucleic acid compositions, poly-glycan compositions and other polymers, lipid and/or cholesterol-based - nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010.053572 and WO 2012/170930, all of which are incorporated herein by reference.

In certain embodiments, the vector described herein is a “replication-defective virus” or a “viral vector” which refers to a synthetic or artificial viral particle in which an expression cassette containing a nucleic acid sequence encoding a functional gene product and the DRG-detargeting miRNA target sequence(s) packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless” -containing only the nucleic acid sequence encoding flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant viral vector is any suitable viral vector. The examples provide illustrative recombinant adeno-associated viruses (rAAV). Other suitable viral vectors may include, e.g., an adenovirus, a poxvirus, a bocavirus, a hybrid AAV/bocavirus, a herpes simplex virus, or a lentivirus. In preferred embodiments, these recombinant viruses are replication incompetent.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

As used herein, the term “target cell” refers to any target cell in which expression of the functional gene product is desired. Examples of target cells may include, but are not limited to, a liver cell, a kidney cell, a cell of the central nervous system, a neuron, a glial cell, and a stem cell. In certain embodiments, the vector is delivered to a target cell ex vivo. In certain embodiments, the vector is delivered to the target cell in vivo.

As used herein, a “vector genome” refers to the nucleic acid sequence packaged inside a viral vector. In one example, a “vector genome” contains, at a minimum, from 5′ to 3′, a vector-specific sequence, a nucleic acid sequence encoding a functional gene product operably linked to regulatory control sequences which direct it expression in a target cell and miRNA target sequences in the untranslated region(s) and a vector-specific sequence. For example, an AAV vector genome contains inverted terminal repeat sequences and an expression cassette which comprises, e.g., a nucleic acid sequence encoding a functional gene product operably linked to regulatory control sequences which direct it expression in a target cell and miRNA target sequences in the untranslated region(s). As described herein, the miRNA target sequences are designed to be specifically recognized by miRNA sequences in cells in which transgene expression is undesirable (e.g., dorsal root ganglia) and/or reduced levels of transgene expression are desired.

It should be understood that the description of the vectors is intended to be applied to other compositions, regiments, aspects, embodiments and methods described across the Specification.

### 3. Adeno-Associated Virus (AAV)

In one aspect, provided herein is a recombinant AAV (rAAV) comprising an AAV capsid and a vector genome packaged therein.

In certain embodiments, the vector genome comprises an AAV 5′ inverted terminal repeat (ITR), an expression cassette as described herein, and an AAV 3′ ITR. In one embodiment, the vector genome refers to the nucleic acid sequence packaged inside a rAAV capsid forming an rAAV vector. Such a nucleic acid sequence contains AAV inverted terminal repeat sequences (ITRs) flanking an expression cassette. In one example, a “vector genome” contains, at a minimum, from 5′ to 3′, an AAV 5′ ITR, a nucleic acid sequence encoding a functional gene product operably linked to regulatory control sequences which direct it expression in a target cell and miRNA target sequences in the untranslated region(s) and an AAV 3′ ITR. In certain embodiments, the ITRs are from AAV2 and the capsid is from a different AAV. Alternatively, other ITRs may be used. As described herein, the miRNA target sequences are designed to be specifically recognized by miRNA sequences in cells in which transgene expression is undesirable and/or reduced levels of transgene expression are desired.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (AITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, a coding sequence and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, full-length AAV 5′ ITR and AAV 3′ ITR are used. In certain embodiments, the vector genome includes a shortened 5′ and/or 3′ AAV2 ITR of 130 base pairs, wherein the external “a” element is deleted. The shortened ITR is reverted back to the wild-type length of 145 base pairs during vector DNA amplification using the internal A element as a template.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., U.S. Published Pat. Application No. 2007-0036760-A1; U.S. Published Pat. Application No. 2009-0197338-A1; EP 1310571. See also, PCT/US19/19861, filed Feb. 27, 2019, and PCT/US19/19804, filed Feb. 27, 2019. See also, WO 2003/042397 (AAV7 and other simian AAV), U.S. Pat. 7790449 and U.S. Pat. 7282199 (AAV8), WO 2005/033321 and US 7,906,111 (AAV9), and WO 2006/110689, and WO 2003/042397 (rh.10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV6.2, AAV7, AAV8, AAV9, AAV8bp, AAVrh10, AAVhu37, AAV7M8 and AAVAnc80, AAVrh90 (PCTUS20/30273, filed Apr. 28, 2020), AAVrh91 (PCTUS20/30266, filed Apr. 28, 2020), and AAVrh92, rh93, and rh91.93 (PCTUS20/30281, filed Apr. 28, 2020), and variants of any of the known or mentioned AAVs or AAVs yet to be discovered or variants or mixtures thereof. See, e.g., WO 2005/033321, which is incorporated herein by reference. In one embodiment, the AAV capsid is an AAV9 capsid or variant thereof. In certain embodiments, the capsid protein is designated by a number or a combination of numbers and letters following the term “AAV” in the name of the rAAV vector.

As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9 % identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV” and “artificial AAV” used interchangeably, mean, without limitation, a AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV⅖ and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, “AAV9 capsid” refers to the AAV9 having the amino acid sequence of (a) GenBank accession: AAS99264, is incorporated by reference herein and the AAV vp1 capsid protein is reproduced in SEQ ID NO: 17, and/or (b) the amino acid sequence encoded by the nucleotide sequence of GenBank Accession: AY530579.1: (nt 1...2211) (reproduced in SEQ ID NO: 16). Some variation from this encoded sequence is encompassed by the present invention, which may include sequences having about 99% identity to the referenced amino acid sequence in GenBank accession: AAS99264 and US7906111 (also WO 2005/033321) (i.e., less than about 1% variation from the referenced sequence). Such AAV may include, e.g., natural isolates (e.g., hu68, hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in US 9,102,949, US 8,927,514, US2015/349911; WO 2016/049230A11; US 9,623,120; US 9,585,971. However, in other embodiments, other variants of AAV9, or AAV9 capsids having at least about 95% identity to the above-referenced sequences may be selected. See, e.g., U.S. Published Pat. Application No. 2015/0079038. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

AAVhu68 varies from another Clade F virus AAV9 by two encoded amino acids at positions 67 and 157 of vp1, SEQ ID NO: 9. In contrast, the other Clade F AAV (AAV9, hu31, hu31) have an Ala at position 67 and an Ala at position 157. Provided are novel AAVhu68 capsids and/or engineered AAV capsids having valine (Val or V) at position 157 based on the numbering of SEQ ID NO: 9 and optionally, a glutamic acid (Glu or E) at position 67. See, also, WO 2018/160582, which is incorporate by reference herein in its entirety (which includes the sequence listing).

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 Jun; 78(10: 6381-6388, which identifies Clades A, B, C, D, E and F, and provides nucleic acid sequences of novel AAV, GenBank Accession Numbers AY530553 to AY530629. See, also, WO 2005/033321.

In certain embodiments, an AAVhu68 capsid is further characterized by one or more of the following. AAV hu68 capsid proteins comprise: AAVhu68 vp1 proteins produced by expression from a nucleic acid sequence which encodes the predicted amino acid sequence of 1 to 736 of SEQ ID NO: 9, vp1 proteins produced from SEQ ID NO: 8, or vp1 proteins produced from a nucleic acid sequence at least 70% identical to SEQ ID NO: 8 which encodes the predicted amino acid sequence of 1 to 736 of SEQ ID NO: 9; AAVhu68 vp2 proteins produced by expression from a nucleic acid sequence which encodes the predicted amino acid sequence of at least about amino acids 138 to 736 of SEQ ID NO:9, vp2 proteins produced from a sequence comprising at least nucleotides 412 to 2211 of SEQ ID NO: 8, or vp2 proteins produced from a nucleic acid sequence at least 70% identical to at least nucleotides 412 to 2211 of SEQ ID NO: 8 which encodes the predicted amino acid sequence of at least about amino acids 138 to 736 of SEQ ID NO: 9, and/or AAVhu68 vp3 proteins produced by expression from a nucleic acid sequence which encodes the predicted amino acid sequence of at least about amino acids 203 to 736 of SEQ ID NO: 9, vp3 proteins produced from a sequence comprising at least nucleotides 607 to 2211 of SEQ ID NO: 8, or vp3 proteins produced from a nucleic acid sequence at least 70% identical to at least nucleotides 607 to 2211 of SEQ ID NO: 8 which encodes the predicted amino acid sequence of at least about amino acids 203 to 736 of SEQ ID NO: 9.

The AAVhu68 vp1, vp2 and vp3 proteins are typically expressed as alternative splice variants encoded by the same nucleic acid sequence which encodes the full-length vp1 amino acid sequence of SEQ ID NO: 9 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 9 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 8), or a sequence at least 70% to at least 99% (e.g., at least 85%, at least 90%, at least 95%, at least 97%, at least 98% or at least 99%) identical to SEQ ID NO: 8 which encodes aa 203 to 736 of SEQ ID NO: 9. Additionally, or alternatively, the vp1-encoding and/or the vp2-encoding sequence may be co-expressed with the nucleic acid sequence which encodes the AAVhu68 vp2 amino acid sequence of SEQ ID NO: 9 (about aa 138 to 736) without the vp1-unique region (about aa 1 to about 137), or a strand complementary thereto, the corresponding mRNA or tRNA (nt 412 to 22121 of SEQ ID NO: 8), or a sequence at least 70% to at least 99% (e.g., at least 85%, at least 90%, at least 95%, at least 97%, at least 98% or at least 99%) identical to SEQ ID NO: 8which encodes about aa 138 to 736 of SEQ ID NO: 9.

As described herein, a rAAVhu68 has a rAAVhu68 capsid produced in a production system expressing capsids from an AAVhu68 nucleic acid which encodes the vp1 amino acid sequence of SEQ ID NO: 9, and optionally additional nucleic acid sequences, e.g., encoding a vp 3 protein free of the vp1 and/or vp2-unique regions. The rAAVhu68 resulting from production using a single nucleic acid sequence vp1 produces the heterogeneous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 9. These subpopulations include, at a minimum, deamidated asparagine (N or Asn) residues. For example, asparagines in asparagine - glycine pairs are highly deamidated.

In one embodiment, the AAVhu68 vp1 nucleic acid sequence has the sequence of SEQ ID NO: 8, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 9 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 8). In certain embodiments, also provided is a nucleic acid sequence which encodes the AAVhu68 vp2 amino acid sequence of SEQ ID NO: 9 (about aa 138 to 736) without the vp1-unique region (about aa 1 to about 137), or a strand complementary thereto, the corresponding mRNA or tRNA (nt 412 to 2211 of SEQ ID NO:8).

However, other nucleic acid sequences which encode the amino acid sequence of SEQ ID NO: 9 may be selected for use in producing rAAVhu68 capsids. In certain embodiments, the nucleic acid sequence has the nucleic acid sequence of SEQ ID NO:8 or a sequence at least 70% to 99% identical, at least 75%, at least 80%, at least 85%, at least 90%, at least 95%, at least 97%, at least 99%, identical to SEQ ID NO: 8 which encodes SEQ ID NO: 9. In certain embodiments, the nucleic acid sequence has the nucleic acid sequence of SEQ ID NO: 8 or a sequence at least 70% to 99.%, at least 75%, at least 80%, at least 85%, at least 90%, at least 95%, at least 97%, at least 99%, identical to about nt 412 to about nt 2211 of SEQ ID NO: 8 which encodes the vp2 capsid protein (about aa 138 to 736) of SEQ ID NO: 9. In certain embodiments, the nucleic acid sequence has the nucleic acid sequence of about nt 607 to about nt 2211 of SEQ ID NO: 8 or a sequence at least 70% to 99.%, at least 75%, at least 80%, at least 85%, at least 90%, at least 95%, at least 97%, at least 99%, identical to nt SEQ ID NO: 8 which encodes the vp3 capsid protein (about aa 203 to 736) of SEQ ID NO: 9.

In certain embodiments, the AAVhu68 capsid is produced using a nucleic acid sequence of SEQ ID NO: 8 or a sequence at least 70%, at least 75%, at least 80%, at least 85%, at least 90%, at least 95%, at least 97%, at least 99%, which encodes the vp1 amino acid sequence of SEQ ID NO: 9 with a modification (e.g., deamidated amino acid) as described herein. In certain embodiments, the vp1 amino acid sequence is reproduced in SEQ ID NO: 9.

As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. SEQ ID NO: 9 provides the encoded amino acid sequence of the AAVhu68 vp1 protein. The term “heterogeneous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine - glycine pairs and optionally further comprising other deamidated amino acids, wherein the deamidation results in an amino acid change and other optional modifications. See PCT/US19/19861, filed Feb. 27, 2019, and PCT/US19/19804, filed Feb. 27, 2019, both entitled “Novel Adeno-Associated Virus (AAV) Vectors, AAV Vectors Having Reduced Capsid Deamidation and Uses Therefor,” which are incorporated by reference herein.

As used herein, a “subpopulation” of vp proteins refers to a group of vp proteins which has at least one defined characteristic in common and which consists of at least one group member to less than all members of the reference group, unless otherwise specified.

For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine - glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 based on the numbering of SEQ ID NO: 9 [AAVhu68] may be deamidated based on the total vp1 proteins may be deamidated based on the total vp1, vp2 and vp3 proteins). Such percentages may be determined using 2D-gel, mass spectrometry techniques, or other suitable techniques.

In the AAVhu68 capsid protein, 4 residues (N57, N329, N452, N512) routinely display levels of deamidation >70% and it most cases >90% across various lots. Additional asparagine residues (N94, N253, N270, N304, N409, N477, and Q599) also display deamidation levels up to ~20% across various lots. The deamidation levels were initially identified using a trypsin digest and verified with a chymotrypsin digestion.

The AAVhu68 capsid contains subpopulations within the vp1 proteins, within the vp2 proteins and within the vp3 proteins which have modifications from the predicted amino acid residues in SEQ ID NO:9. These subpopulations include, at a minimum, certain deamidated asparagine (N or Asn) residues. For example, certain subpopulations comprise at least one, two, three or four highly deamidated asparagines (N) positions in asparagine -glycine pairs in SEQ ID NO: 9 and optionally further comprising other deamidated amino acids, wherein the deamidation results in an amino acid change and other optional modifications.

In other embodiments, the method involves increasing yield of a rAAV and thus, increasing the amount of an rAAV which is present in supernatant prior to, or without requiring cell lysis. This method involves engineering an AAV VP1 capsid gene to express a capsid protein having Glu at position 67, Val at position 157, or both based on an alignment having the amino acid numbering of the AAVhu68 vp1 capsid protein. In other embodiments, the method involves engineering the VP2 capsid gene to express a capsid protein having the Val at position 157. In still other embodiments, the rAAV has a modified capsid comprising both vp1 and vp2 capsid proteins Glu at position 67 and Val at position 157.

In certain embodiments, the rAAV as described herein is a self-complementary AAV. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In certain embodiments, the rAAV described herein is nuclease-resistant. Such nuclease may be a single nuclease, or mixtures of nucleases, and may be endonucleases or exonucleases. A nuclease-resistant rAAV indicates that the AAV capsid has fully assembled and protects these packaged genomic sequences from degradation (digestion) during nuclease incubation steps designed to remove contaminating nucleic acids which may be present from the production process. In many instances, the rAAV described herein is DNase resistant.

The recombinant adeno-associated virus (AAV) described herein may be generated using techniques which are known. See, e.g., WO 2003/042397; WO 2005/033321, WO 2006/110689; US 7588772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr 15; 20(R1): R2-R6. Published online 2011 Apr 29. doi: 10.1093/hmg/ddrl41; Aucoin MG et al., Production of adeno-associated viral vectors in insect cells using triple infection: optimization of baculovirus concentration ratios. Biotechnol Bioeng. 2006 Dec 20;95(6): 1081-92; SAMI S. THAKUR, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug 10. pii: S1525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 Feb;28(1): 15-22. doi: 10.1089/hgtb.2016.164.; Li L et al. Production and characterization of novel recombinant adeno-associated virus replicative-form genomes: a eukaryotic source of DNA for gene transfer. PLoS One. 2013 Aug 1;8(8):e69879. doi: 10.1371/joumal.pone.0069879. Print 2013; Galibert L et al, Latest developments in the large-scale production of adeno-associated virus vectors in insect cells toward the treatment of neuromuscular diseases. J Invertebr Pathol. 2011 Jul;107 Suppl: S80-93. doi: 10.1016/j.jip.2011.05.008; and Kotin RM, Large-scale recombinant adeno-associated virus production. Hum Mol Genet. 2011 Apr 15;20(R1):R2-6. doi: 10.1093/hmg/ddrl41. Epub 2011 Apr 29.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, which is incorporated by reference herein. In brief, the method for separating rAAV9 particles having packaged genomic sequences from genome-deficient AAV9 intermediates involves subjecting a suspension comprising recombinant AAV9 viral particles and AAV9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where # of GC = # of particles) are plotted against GC particles loaded. The resulting linear equation (y = mx+c) is used to calculate the number of particles in the band volumes of the test article peaks. The number of particles (pt) per 20 µL loaded is then multiplied by 50 to give particles (pt) /mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer’s instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. Endpoint assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 Apr;25(2): 115-25. doi: 10.1089/hgtb.2013.131. Epub 2014 Feb 14.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 Dec; 87(24): 13150-13160; Buller RM, Rose JA. 1978. Characterization of adenovirus-associated virus-induced polypeptides in KB cells. J. Virol. 25:331-338; and Rose JA, Maizel JV, Inman JK, Shatkin AJ. 1971. Structural proteins of adenovirus-associated viruses. J. Virol. 8:766-770.

It should be understood that the description of the rAAVs is intended to be applied to other compositions, regiments, aspects, embodiments and methods described across the Specification.

### 4. Pharmaceutical Composition

A pharmaceutical composition comprising the expression cassette comprising the transgene and the miRNA target sequences may be a liquid suspension, a lyophilized or frozen composition, or another suitable formulation. In certain embodiments, the composition comprises the expression cassette and a physiologically compatible liquid (e.g., a solution, diluent, carrier) which form a suspension. Such a liquid is preferably aqueous based and may contain one or more: buffering agent (s), a surfactant(s), pH adjuster(s), preservative(s), or other suitable excipients. Suitable components are discussed in more detail below. The pharmaceutical composition comprises the aqueous suspending liquid and any selected excipients, and the expression cassette.

The expression cassette comprising the transgene and the miRNA target sequences is as described throughout this specification herein. For example, an expression cassette may be a nucleic acid sequence comprising: (a) a coding sequence for the gene product under the control of regulatory sequences which direct expression of the gene product in a cell containing the recombinant virus; (b) regulatory sequences which direct expression of the gene product in a cell: (c) a 5′ untranslated region (UTR) sequence which is 5′ of the coding sequence; (d) a 3′ UTR sequence which is 3′ of the coding sequence; and e) at least two tandem dorsal root ganglion (DRG)-specific miRNA target sequences, wherein the at least two miRNA target sequences comprise at least a first miRNA target sequence and at least a second miRNA target sequence which may be the same or different.

In certain embodiments, the pharmaceutical composition comprises the expression cassette comprising the transgene and the miRNA target sequences and a non-viral delivery system. This may include, e.g, naked DNA, naked RNA, an inorganic particle, a lipid or lipid-like particle, a chitosan-based formulation and others known in the art and described for example by Ramamoorth and Narvekar, as cited above).

In other embodiments, the pharmaceutical composition is a suspension comprising the expression cassette comprising the transgene and the miRNA target sequences is engineered in a non-viral or viral vector system. Such a non-viral vector system may include, e.g., a plasmid or non-viral genetic element, or a protein-based vector.

In certain embodiments, the pharmaceutical composition comprises a non-replicating viral vector. Suitable viral vectors may include any suitable delivery vector, such as, e.g., a recombinant adenovirus, a recombinant lentivirus, a recombinant bocavirus, a recombinant adeno-associated virus (AAV), or another recombinant parvovirus. In certain embodiments, the viral vector is a recombinant AAV for delivery of a gene product to a patient in need thereof.

In one embodiment, the pharmaceutical composition comprises the expression cassette comprising the transgene and the miRNA target sequences and a formulation buffer suitable for delivery via intracerebroventricular (ICV), intrathecal (IT), intracisternal or intravenous (IV) injection. In one embodiment, the expression cassette comprising the transgene and the miRNA target sequences is in packaged a recombinant AAV.

In one embodiment, a composition as provided herein comprises a surfactant, preservative, excipients, and/or buffer dissolved in the aqueous suspending liquid. In one embodiment, the buffer is PBS. In another embodiment, the buffer is an artificial cerebrospinal fluid (aCSF), e.g., Eliott’s formulation buffer; or Harvard apparatus perfusion fluid (an artificial CSF with final Ion Concentrations (in mM): Na 150; K 3.0; Ca 1.4; Mg 0.8; P 1.0; Cl 155). Various suitable solutions are known including those which include one or more of buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

Suitably, the formulation is adjusted to a physiologically acceptable pH, e.g., in the range of pH 6 to 8, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other routes of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits x 100 give the approximate molecular mass of the polyoxypropylene core, and the last digit x 10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate ·7H2O), potassium chloride, calcium chloride (e.g., calcium chloride ·2H2O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B® solution [Lukare Medical].

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA

Additionally provided is a pharmaceutical composition comprising a pharmaceutically acceptable carrier and a vector comprising a nucleic acid sequence as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells. In particular, the rAAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

The aqueous suspension or pharmaceutical compositions described herein are designed for delivery to subjects in need thereof by any suitable route or a combination of different routes.

In one embodiment, the pharmaceutical composition is formulated for delivery via intracerebroventricular (ICV), intrathecal (IT), or intracisternal injection. In one embodiment, the compositions described herein are designed for delivery to subjects in need thereof by intravenous injection. Alternatively, other routes of administration may be selected (e.g., oral, inhalation, intranasal, intratracheal, intraarterial, intraocular, intramuscular, and other parenteral routes).

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, suboccipital/intracistemal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

In one aspect, provided herein is a pharmaceutical composition comprising a vector as described herein in a formulation buffer. In certain embodiments, the replication-defective virus compositions can be formulated in dosage units to contain an amount of replication-defective virus that is in the range of about 1.0 x 10⁹ GC to about 1.0 x 10¹⁶ GC (to treat an average subject of 70 kg in body weight) including all integers or fractional amounts within the range, and preferably 1.0 x 10¹² GC to 1.0 x 10¹⁴ GC for a human patient. In one embodiment, the compositions are formulated to contain at least 1x10⁹, 2x10⁹, 3x10⁹, 4x10⁹, 5x10⁹, 6x10⁹, 7x10⁹, 8x10⁹, or 9x10⁹ GC per dose including all integers or fractional amounts within the range. In another embodiment, the compositions are formulated to contain at least 1x10¹⁰, 2x10¹⁰, 3x10¹⁰, 4x10¹⁰, 5x10¹⁰, 6x10¹⁰, 7x10¹⁰, 8x10¹⁰, or 9x10¹⁰ GC per dose including all integers or fractional amounts within the range. In another embodiment, the compositions are formulated to contain at least 1x10¹¹, 2x10¹¹, 3x10¹¹, 4x10¹¹, 5x10¹¹, 6x10¹¹, 7x10¹¹, 8x10¹¹, or 9x10¹¹ GC per dose including all integers or fractional amounts within the range. In another embodiment, the compositions are formulated to contain at least 1x10¹², 2x10¹², 3x10¹², 4x10¹², 5x10¹², 6x10¹², 7x10¹², 8x10¹², or 9x10¹² GC per dose including all integers or fractional amounts within the range. In another embodiment, the compositions are formulated to contain at least 1x10¹³, 2x10¹³, 3x10¹³, 4x10¹³, 5x10¹³, 6x10¹³, 7x10¹³, 8x10¹³, or 9x10¹³ GC per dose including all integers or fractional amounts within the range. In another embodiment, the compositions are formulated to contain at least 1x10¹⁴, 2x10¹⁴, 3x10¹⁴, 4x10¹⁴, 5x10¹⁴, 6x10¹⁴, 7x10¹⁴, 8x10¹⁴, or 9x10¹⁴ GC per dose including all integers or fractional amounts within the range. In another embodiment, the compositions are formulated to contain at least 1x10¹⁵, 2x10¹⁵, 3x10¹⁵, 4x10¹⁵, 5x10¹⁵, 6x10¹⁵, 7x10¹⁵, 8x10¹⁵, or 9x10¹⁵ GC per dose including all integers or fractional amounts within the range. In one embodiment, for human application the dose can range from 1x10¹⁰ to about 1x10¹² GC per dose including all integers or fractional amounts within the range.

In one embodiment, provided is a pharmaceutical composition comprising a rAAV as described herein in a formulation buffer. In one embodiment, the rAAV is formulated at about 1 x 10⁹ genome copies (GC)/mL to about 1 x 10¹⁴ GC/mL. In a further embodiment, the rAAV is formulated at about 3 x 10⁹ GC/mL to about 3 x 10¹³ GC/mL. In yet a further embodiment, the rAAV is formulated at about 1 x 10⁹ GC/mL to about 1 x 10¹³ GC/mL. In one embodiment, the rAAV is formulated at least about 1 x 10¹¹ GC/mL. In one embodiment, the pharmaceutical composition comprising a rAAV as described herein is administrable at a dose of about 1 x 10⁹ GC per gram of brain mass to about 1 x 10¹⁴ GC per gram of brain mass.

In certain embodiments, the composition may be formulated in a suitable aqueous suspension media (e.g., a buffered saline) for delivery by any suitable route. The compositions provided herein are useful for systemic delivery of high doses of viral vector. For rAAV, a high dose may be at least 1 x10¹³ GC or at least 1 x10¹⁴ GC. However, for improved safety, the miRNA sequences provided herein may be included in expression cassettes and/or vector genomes which are delivered at other lower doses.

In certain embodiments, the composition is delivered by two different routes at essentially the same time.

It should be understood that the description of the pharmaceutical compositions is intended to be applied to other compositions, regimens, aspects, embodiments and methods described across the Specification.

### 5. Method of Treatment

In certain embodiments, the compositions provided herein are useful for delivery of a desired transgene product to patient, while for repressing transgene expression in dorsal root ganglion neurons. The method involves delivering a composition comprising an expression cassette comprising the transgene and miRNA target sequences to a patient. Useful transgenes include those that encode a variety of gene products that replace a defective or deficient gene, inactivate or “knock-out”, or “knock-down” or reduce the expression of a gene that is expressing at an undesirably high level, or delivering a gene product that has a desired therapeutic effect.

Suitably, the methods of treatment comprise dosing a patient with vectors comprising an expression cassette or vector genomes comprising the transgenes described in this specification in combination with multiple miR target sequences described herein. Suitably, these expression cassettes and vector genomes are packaged into a suitable viral (e.g., AAV) capsid. In certain embodiments, the expression cassette comprises eight miR targeting sequences (e.g, 4x miR-182 targeting sequences + 4x miR-183 targeting sequences, or other combinations) may be generated. In other embodiments, various combinations of miR-targeting sequences may be generated.

Examples of suitable transgenes useful in treatment of one or more neurodegenerative disorders. Such disorders may include, without limitation, transmissible spongiform encephalopathies (e.g., Creutzfeld-Jacob disease), Duchenne muscular dystrophy (DMD), myotubular myopathy and other myopathies, Parkinson’s disease, amyotrophic lateral sclerosis (ALS), multiple sclerosis, Alzheimer’s Disease, Huntington disease, Canavan’s disease, traumatic brain injury, spinal cord injury (ATI335, anti-nogol by Novartis), migraine (ALD403 by Alder Biopharmaceuticals; LY2951742 by Eli; RN307 by Labrys Biologics), lysosomal storage diseases, stroke, and infectious disease affecting the central nervous system. Examples of lysosomal storage disease include, e.g., Gaucher disease, Fabry disease, Niemann-Pick disease, Hunter syndrome, glycogen storage disease II (Pompe disease), or Tay-Sachs disease. For certain of these conditions, e.g., DMD and myopathies, the compositions provided herein are useful in reducing or eliminating axonopathy associated with high doses of expression cassettes (e.g., carried by a viral vector) for transduction or invention of skeletal and cardiac muscle.

Still other nucleic acids may encode an immunoglobulin which is directed to leucine rich repeat and immunoglobulin-like domain-containing protein 1 (LINGO-1), which is a functional component of the Nogo receptor and which is associated with essential tremors in patients which multiple sclerosis, Parkinson’s Disease or essential tremor. One such commercially available antibody is ocrelizumab (Biogen, BIIB033). See, e.g., U.S. Pat. 8,425,910. In one embodiment, the nucleic acid constructs encode immunoglobulin constructs useful for patients with ALS. Examples of suitable antibodies include antibodies against the ALS enzyme superoxide dismutase 1 (SOD1) and variants thereof (e.g., ALS variant G93A, C4F6 SOD1 antibody); MS785, which directed to Derlin-1-binding region); antibodies against neurite outgrowth inhibitor (NOGO-A or Reticulon 4), e.g., GSK1223249, ozanezumab (humanized, GSK, also described as useful for multiple sclerosis). Nucleic acid sequences may be designed or selected which encode immunoglobulins useful in patients having Alzheimer’s Disease. Such antibody constructs include, e.g., adumanucab (Biogen), Bapineuzumab (Elan; a humanised mAb directed at the amino terminus of Aβ); Solanezumab Eli Lilly, a humanized mAb against the central part of soluble Aβ); Gantenerumab (Chugai and Hoffmann-La Roche, is a full human mAb directed against both the amino terminus and central portions of Aβ); Crenezumab (Genentech, a humanized mAb that acts on monomeric and conformational epitopes, including oligomeric and protofibrillar forms of Aβ; BAN2401 (Esai Co., Ltd, a humanized immunoglobulin G1 (IgGl) mAb that selectively binds to Aβ protofibrils and is thought to either enhance clearance of Aβ protofibrils and/or to neutralize their toxic effects on neurons in the brain); GSK 933776 (a humanised IgGl monoclonal antibody directed against the amino terminus of Aβ); AAB-001, AAB-002, AAB-003 (Fc-engineered bapineuzumab); SAR228810 (a humanized mAb directed against protofibrils and low molecular weight Aβ); BIIB037/BART (a full human IgGl against insoluble fibrillar human Aβ, Biogen Idec), an anti-Aβ antibody such m266, tg2576 (relative specificity for Aβ oligomers) [Brody and Holtzman, Annu Rev Neurosci, 2008; 31: 175-193]. Other antibodies may be targeted to beta-amyloid proteins, Aβ, beta secretase and/or the tau protein. In still other embodiments, an anti-β-amyloid antibody is derived from an IgG4 monoclonal antibodies to target β-amyloid in order to minimize effector functions, or construct other than an scFv which lacks an Fc region is selected in order to avoid amyloid related imaging abnormality (ARIA) and inflammatory response. In certain of these embodiments, the heavy chain variable region and/or the light chain variable region of one or more of the scFv constructs is used in another suitable immunoglobulin construct as provided herein. These scFV and other engineered immunoglobulins may reduce the half-life of the immunoglobulin in the serum, as compared to immunoglobulins containing Fc regions. Reducing the serum concentration of anti-amyloid molecules may further reduce the risk of ARIA, as extremely high levels of anti-amyloid antibodies in serum may destabilize cerebral vessels with a high burden of amyloid plaques, causing vascular permeability. Nucleic acids encoding other immunoglobulin constructs for treatment of patients with Parkinson’s disease may be engineered or designed to express constructs, including, e.g., leucine-rich repeat kinase 2, dardarin (LRRK2) antibodies,; anti-synuclein and alpha-synuclein antibodies and DJ-1 (PARK7) antibodies,. Other antibodies may include, PRX002 (Prothena and Roche) Parkinson’s disease and related synucleinopathies. These antibodies, particularly anti-synuclein antibodies may also be useful in treatment of one or more lysosomal storage disease.

One may engineer or select nucleic acid constructs encoding an immunoglobulin construct for treating conditions associated with central nervous system (CNS) disorders or diseases including, e.g, multiple sclerosis, Parkinson’s disease, Alzheimer’s disease, ALS or various cancers, which are engineered into expression cassettes containing the miR-182 target and/or miR-183 target sequences as provided herein in order to reduce or prevent drg toxicity. Such immunoglobulins may include or be derived from antibodies such as natalizumab (a humanized anti-a4-ingrin, iNATA, Tysabri, Biogen Idec and Elan Pharmaceuticals), which was approved in 2006, alemtuzumab (Campath®-1H, a humanized anti-CD52), rituximab (Rituxin®, a chimeric anti-CD20), daclizumab (Zenepax, a humanized anti-CD25), ocrelizumab (humanized, anti-CD20, Roche), ustekinumab (CNTO-1275, a human anti-IL12 p40+IL23p40); anti-LINGO-1, an anti-CD30 antibody (e.g., brentuximab - vedotin (Adcentris®)); and ch5D12 (a chimeric anti-CD40), and rHIgM22 (a remyelinated monoclonal antibody; Acorda and the Mayo Foundation for Medical Education and Research). Still other anti-a4-integrin antibodies, anti-CD20 antibodies (e.g., ofatumumab (Arzerra®), Gaztvaro®, Gazwa/Obinutuzumab), Mabthera®, anti-CD52 antibodies, anti-VEGF or anti-VEGF2 antibodies (e.g., Cyramza® (ramucirumab)), anti-CD38 (e.g., Darzalex® (daratumumab), anti-EGFR (e.g., Erbitux® (cetuximab) or Vectibix® (panitumumab)), anti-Her2, e.g., trastuzumab or pertuzumab, anti-PDl (eg., nivolumab), anti-RANKL (e.g., denosumab), anti-PD-L1 (eg., atezolizumab), anti-EGFR (e.g., panitumumab), anti-CTLA4 (e.g., ipilimumab), anti-IL17, anti-CD19, anti-SEMA4D, and anti-CD40 antibodies may be delivered via the AAV vectors as described herein. Still other immunoglobulin constructs or monoclonal antibodies may be selected for use in the invention. See, e.g., US2018/0339065, which is incorporated by reference herein. Antibodies may be CNS-targeted or delivered via other routes.

Antibodies against various infections of the central nervous system is also contemplated by the present invention. Such infectious diseases may include fungal diseases such as cryptoccocal meningitis, brain abscess, spinal epidural infection caused by, e.g., Cryptococcus neoformans, Coccidioides immitis, order Mucorales, Aspergillus spp, and Candida spp; protozoal, such as toxoplasmosis, malaria, and primary amoebic meningoencephalitis, caused by agents such as, e.g., Toxoplasma gondii, Taenia solium, Plasmodium falciparus, Spirometra mansonoides (sparaganoisis), Echinococcus spp (causing neuro hydatosis), and cerebral amoebiasis; bacterial, such as, e.g., tuberculosis, leprosy, neurosyphilis, bacterial meningitis, lyme disease (Borrelia burgdorferi), Rocky Mountain spotted fever (Rickettsia rickettsia), CNS nocardiosis (Nocardia spp), CNS tuberculosis (Mycobacterium tuberculosis), CNS listeriosis (Listeria monocytogenes), brain abscess, and neuroborreliosis; viral infections, such as, e.g., viral meningitis, Eastern equine encephalitis (EEE), St Louis encepthalitis, West Nile virus and/or encephalitis, rabies, California encephalitis virus, La Crosse encepthalitis, measles encephalitis, poliomyelitis, which may be caused by, e.g., herpes family viruses (HSV), HSV-1, HSV-2 (neonatal herpes simplex encephalitis), varicella zoster virus (VZV), Bickerstaff encephalitis, Epstein-Barr virus (EBV), cytomegalovirus (CMV, such as TCN-202 is in development by Theraclone Sciences), human herpesvirus 6 (HHV-6), B virus (herpesvirus simiae), Flavivirus encephalitis, Japanese encephalitis, Murray valley fever, JC virus (progressive multifocal leukoencephalopathy), Nipah Virus (NiV), measles (subacute sclerosing panencephalitis); and other infections, such as, e.g., subactuate sclerosing panencephalitis, progressive multifocal leukoencephalopathy; human immunodeficiency virus (acquired immunodeficiency syndrome (AIDS)); streptococcus pyogenes and other β- hemolytic Streptococcus (e.g., Pediatric Autoimmune Neuropsychiatric Disorders Associated with Streptococcal Infection, PANDAS) and/or Syndenham’s chorea, and Guillain-Barre syndrome, and prions.

Examples of suitable antibody constructs may include those described, e.g., in WO 2007/012924A2, Jan. 29, 2015, which is incorporated by reference herein.

For example, other nucleic acid sequences comprising the drg-targeting sequences provided herein may be operably linked to sequences which encode anti-prion immunoglobulin constructs. Such immunoglobulins may be directed against major prion protein (PrP, for prion protein or protease-resistant protein, also known as CD230 (cluster of differentiation 230). The amino acid sequence of PrP is provided, e.g., http://www.ncbi.nlm.nih.gov/protein/NP_000302, incorporated by reference herein. The protein can exist in multiple isoforms, the normal PrPC, the disease-causing PrPSc, and an isoform located in mitochondria. The misfolded version PrPSc is associated with a variety of cognitive disorders and neurodegenerative diseases such as Creutzfeldt-Jakob disease, bovine spongiform encephalopathy, Gerstmann-Straussler-Scheinker syndrome, fatal familial insomnia, and kuru.

Examples of suitable gene products may include those associated expressed from vector genomes comprising the miR-182/miR-183 targeting sequences provided herein operably linked to coding sequences for a therapeutic gene(s) useful for treatment with familial hypercholesterolemia, muscular dystrophy, cystic fibrosis, and rare or orphan diseases. Examples of such rare disease may include spinal muscular atrophy (SMA), Huntingdon’s Disease, Rett Syndrome (e.g., methyl-CpG-binding protein 2 (MeCP2) UniProtKB - P51608); Angelman’s Disease (e.g., ubiquitin-protein ligase E3A (UBE3A), also known as E6AP, Amyotrophic Lateral Sclerosis (ALS), Duchenne Type Muscular dystrophy, Friedrichs Ataxia (e.g., frataxin), progranulin (PRGN) (associated with non-Alzheimer’s cerebral degenerations, including, frontotemporal dementia (FTD), progressive non-fluent aphasia (PNFA) and semantic demential), among others. Other useful gene products include, carbamoyl synthetase I, omithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha- fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)).

Further illustrative genes which may be delivered via the rAAV containing vector genome with the miR targeting sequences provided herein operably linked to a gene selected from, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase, associated with phenylketonuria (PKU); branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; a methylmalonic acidemia (MMA); Niemann-Pick disease, type C1); propionic academia (PA); low density lipoprotein receptor (LDLR) protein, associated with familial hypercholesterolemia (FH); UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); ATP7B associated with Wilson’s Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson’s disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes. Additional genes and diseases of interest include, e.g., dystonin gene related diseases such as Hereditary Sensory and Autonomic Neuropathy Type VI (the DST gene encodes dystonin; dual AAV vectors may be required due to the size of the protein (~7570 aa); SCN9A related diseases, in which loss of function mutants cause inability to feel pain and gain of function mutants cause pain conditions, such as erythromelagia. Another condition is Charcot-Marie-Tooth type 1F and 2E due to mutations in the NEFL gene (neurofilament light chain). characterized by a progressive peripheral motor and sensory neuropathy with variable clinical and electrophysiologic expression. In certain embodiments, the vectors described herein may be used in treatment of mucopolysaccaridoses (MPS) disorders. Such vectors may contain carry a nucleic acid sequence encoding α-L-iduronidase (IDUA) for treating MPS I (Hurler, Hurler-Scheie and Scheie syndromes); a nucleic acid sequence encoding iduronate-2-sulfatase (IDS) for treating MPS II (Hunter syndrome); a nucleic acid sequence encoding sulfamidase (SGSH) for treating MPSIII A, B, C, and D (Sanfilippo syndrome); a nucleic acid sequence encoding N-acetylgalactosamine-6-sulfate sulfatase (GALNS) for treating MPS IV A and B (Morquio syndrome); a nucleic acid sequence encoding arylsulfatase B (ARSB) for treating MPS VI (Maroteaux-Lamy syndrome); a nucleic acid sequence encoding hyaluronidase for treating MPSI IX (hyaluronidase deficiency) and a nucleic acid sequence encoding beta-glucuronidase for treating MPS VII (Sly syndrome). See, e.g., www.orpha.net/consor/cgi-bin/Disease_Search_List.php; rarediseases.info.nih.gov/diseases.

Examples of other suitable genes which may be in an expression cassette or vector genome operably linked to the miR-targeting sequences may include, e.g., hormones and growth and differentiation factors including, without limitation, insulin, glucagon, glucagon-like peptide -1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-⅘, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1α, IL-1β, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-CD20, anti-NGF, anti-GnRH. Useful gene products also include complement regulatory proteins such as complement regulatory proteins, membrane cofactor protein (MCP), decay accelerating factor (DAF), CR1, CF2, CD59, and C1 esterase inhibitor (C1-INH). Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

The drg-detargeting sequences may also be used in delivery vectors for gene editing. The drg-detargeting sequence may be delivered downstream of a nuclease. In one embodiment, the coding sequence encodes a nuclease selected from a meganuclease, a zinc finger nuclease, a transcription activator-like (TAL) effector nuclease (TALEN), and a clustered, regularly interspaced short palindromic repeat (CRISPR)/endonuclease (Cas9, Cpf1, etc). Examples of suitable meganucleases are described, e.g., in U.S. Pat. 8,445,251; US 9,340,777; US 9,434,931; US 9,683,257, and WO 2018/195449. Other suitable enzymes include nuclease-inactive S. pyogenes CRISPR/Cas9 that can bind RNA in a nucleic-acid-programmed manner (Nelles et al, Programmable RNA Tracking in Live Cells with CRISPR/Cas9, Cell, 165(2):P488-96 (April 2016)), and base editors (e.g., Levy et al. Cytosine and adenine base editing of the brain, liver, retina, heart and skeletal muscle of mice via adeno-associated viruses, Nature Biomedical Engineering, 4, 97-110 (January 2020)). In certain embodiments, the nuclease is not a zinc finger nuclease. In certain embodiments, the nuclease is not a CRISPR-associated nuclease. In certain embodiments, the nuclease is not a TALEN. In one embodiment, the nuclease is not a meganuclease. In certain embodiments, the nuclease is a member of the LAGLIDADG (SEQ ID NO: 24) family of homing endonucleases. In certain embodiments, the nuclease is a member of the I-CreI family of homing endonucleases which recognizes and cuts a 22 base pair recognition sequence SEQ ID NO: 25 - CAAAACGTCGTGAGACAGTTTG. See, e.g., WO 2009/059195. Methods for rationally-designing mono-LAGLIDADG homing endonucleases were described which are capable of comprehensively redesigning ICreI and other homing endonucleases to target widely-divergent DNA sites, including sites in mammalian, yeast, plant, bacterial, and viral genomes (WO 2007/047859).

Suitable gene editing targets include, e.g., liver-expressed genes such as, without limitation, proprotein convertase subtilisin/kexin type 9 (PCSK9) (cholesterol related disorders), transthyretin (TTR) (transthyretin amyloidosis), HAO, apolipoprotein C-III(APOC3), Factor VIII, Factor IX, low density lipoprotein receptor (LDLr), lipoprotein lipase (LPL) (Lipoprotein Lipase Deficiency), lecithin-cholesterol acyltransferase (LCAT), ornithine transcarbamylase (OTC), carnosinase (CN1), sphingomyelin phosphodiesterase (SMPD1) (Niemann-Pick disease), hypoxanthine-guanine phosphoribosyltransferase (HGPRT), branched-chain alpha-keto acid dehydrogenase complex (BCKDC) (maple syrup urine disease), erythropoietin (EPO), Carbamyl Phosphate Synthetase (CPS1), N-Acetylglutamate Synthetase (NAGS), Argininosuccinic Acid Synthetase (Citrullinemia), Argininosuccinate Lyase (ASL) (Argininosuccinic Aciduria), and Arginase (AG).

Other editing gene targets may include, e.g., hydroxymethylbilane synthase (HMBS), carbamoyl synthetase I, ornithine transcarbamylase (OTC), arginosuccinate synthetase, arginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase. glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson’s Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; Nieman Pick disease type B, Nieman Pick disease tupe c, argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson’s disease; PRKN for the treatment of Parkinson’s disease, the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

Methods for sequencing a protein, peptide, or polypeptide (e.g., as an immunoglobulin) are known to those of skill in the art. Once the sequence of a protein is known, there are web-based and commercially available computer programs, as well as service based companies which back translate the amino acids sequences to nucleic acid coding sequences. See, e.g., backtranseq by EMBOSS, available at www.ebi.ac.uk/Tools/st/ ; Gene Infinity, available at geneinfinity.org/sms/sms_backtranslation.html); ExPasy, available at expasy.org/tools/. In one embodiment, the RNA and/or cDNA coding sequences are designed for optimal expression in human cells.

In certain embodiments, the compositions provided herein are useful for a method for modulating neuronal degeneration and/or decrease secondary dorsal spinal cord axonal degeneration following intrathecal or systemic gene therapy administration. Thus, while the compositions provided herein are particularly useful for delivery of gene therapy to the CNS, they may also be useful for other routes of delivery, including e.g. systemic IV delivery, where high doses of the gene therapy may result in DRG transduction and toxicity. The method involves delivering a composition comprising an expression cassette or vector genome comprising the transgene and miRNA target(s) to a patient.

In certain embodiments, the compositions provided herein are useful in methods for repressing transgene expression in the DRG. In certain embodiments, the method comprises delivering an expression cassette or vector genome that includes a miR-183 target sequence to repress transgene expression levels in the DRG. In certain embodiments, the method comprises delivering an expression cassette or vector genome useful for repressing transgene expression in the DRG, wherein the expression cassette or vector genome includes at least two miR-183 target sequences, at least three miR-183 target sequences, at least four miR-183 target sequences, at least five miR-183 target sequences, at least six miR-183 target sequences, or at least seven miR-183 target sequences. In certain embodiments, the method comprises delivering an expression cassette or vector genome useful for repressing transgene expression in the DRG, wherein the expression cassette or vector genome comprises eight miR-183 target sequences. In certain embodiments, the method enhances expression in one or more cells present in the CNS selected from one or more of pyramidal neurons, purkinje neurons, granule cells, spindle neurons, interneuron cells, astrocytes, oligodendrocytes, microglia, and/or ependymal cells.

In certain embodiments, provided is a method useful for delivering and/or enhancing expression of transgene in lower motor neurons the retina, inner ear, and olfactory receptors comprising delivering an expression cassette or vector genome that includes a transgene operably linked to one or more miR-183 target sequences and/or more miR-183 target sequences. In certain embodiments, the cells or tissues may be one or more of liver, or heart.

In yet another embodiment, provided is a method comprising delivering an expression cassette or vector genome to cells present in the CNS wherein the expression cassette or vector genome comprises one or more miR-183 target sequences and lacks a transgene (i.e. a sequence encoding a heterologous gene product). In such embodiments, delivery of miR-183 to cells of the CNS is achieved. In certain embodiments, delivery of an expression cassette or vector genome comprising miR-183 sequences results in repression of DRG expression and enhanced gene expression in certain other cells present in the CNS.

In certain embodiments, the compositions provided herein are useful in methods for enhancing expression of a transgene in a cell outside the CNS. In certain embodiments, methods for enhancing expression in a cell outside the CNS comprise delivering an expression cassette or vector genome that includes a miR-182 target sequence to a patient.

In one embodiment, the suspension has a pH of about 6.8 to about 7.32.

Suitable volumes for delivery of these doses and concentrations may be determined by one of skill in the art. For example, volumes of about 1 µL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In one embodiment, the composition comprising an rAAV as described herein is administrable at a dose of about 1 × 10⁹ GC per gram of brain mass to about 1 × 10¹⁴ GC per gram of brain mass. In certain embodiments, the rAAV is co-administered systemically at a dose of about 1 × 10⁹ GC per kg body weight to about 1 × 10¹³ GC per kg body weight

In one embodiment, the subject is delivered a therapeutically effective amount of the expression cassettes described herein. As used herein, a “therapeutically effective amount” refers to the amount of the expression cassette comprising the nucleic acid sequence encoding the gene product and the miRNA target sequences which delivers and expresses in the target cells and which specifically detargets DRG expression.

The use of rAAV for delivering for the treatment of various conditions have been previously described. The expression cassettes for these rAAVs can be modified to include eight miRNA target sequences described herein (including, e.g., miR-183 target sequences, miR-182 target sequences, or combinations thereof) to, for example, reduce transgene expression in DRG and/or reduce or eliminate DRG toxicity and/or axonopathy. Examples of rAAV vector genomes that can be modified to include miRNA target sequences include the genes described in WO 2017/136500 (MPSI), WO 2017/181113 (MPSII), WO 2019/108857 (MPSIIIA), WO 2019/108856 (MPSIIIB), MPSIV, MPSVII, WO 2017/106354 (SMN1), WO 2018/160585 (SMN1), Batten’s disease as caused by CLN1, CLN2, CLN3, CLN4, CLN5, CLN6, CLN8 (see, e.g., WO 2018/209205 (Batten disease), WO 2015/164723 (AAV-mediated delivery of anti-HER2 antibody),

Other suitable transgenes may include, e.g., WO2015/138348 (OTC), WO 2015/164778 (LDLR variants for FH); WO2017/106345 (Crigler-Najjar), WO 2017/106326 (anti-PCSK9 Abs), WO 2017/180857 (hemophilia A, Factor VIII), WO 2017/180861 (hemophilia B, Factor IX), as well as vectors in trials for treatment of Myotubular Myopathy (such as AT132, AAV8, Audentes).

Expression cassettes or vector genomes comprising the transgenes described in this specification and multiple miR target sequences may be generated as described herein. In certain embodiments, the expression cassette comprising eight miR targeting sequences (e.g, 4x miR-182 targeting sequences + 4x miR-183 targeting sequences, or other combinations) are generated. In other embodiments, various combinations of miR-targeting sequences may be generated. Suitably, these expression cassettes and vector genomes are packaged into a suitable viral (e.g., AAV) capsid.

In certain embodiments, an AAV.alpha-L-iduronidase (AAV.IDUA) gene therapy vector comprises a vector genome comprising eight miR target sequences of the miRNA183 cluster (including miR-182 target sequences, and miR-183 target sequences, or combinations thereof) operably linked to the coding sequence for the IDUA gene (see, e.g., nt 1938-3908 of SEQ ID NO: 15). In certain embodiments, the vector genome comprises multiple copies of the same miR target sequence each separated by a spacer which may be the same or which may differ from each other. In another embodiment, the vector genome comprises multiple copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.iduronate-2-sulfatase (IDS) (AAV.IDS) gene therapy vector comprises a vector genome comprising eight miR target sequences of the miR-183 cluster (including miR-182 target sequences, and miR-183 target sequences, or combinations thereof) operably linked to the coding sequence for the IDS gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.N-sulfoglucosamine sulfohydrolase (AAV.SGSH) gene therapy vector comprises a vector genome comprising eight miR target sequences of the miR-183 cluster (including miR-182 target sequences, and miR-183 target sequences, or combinations thereof) operably linked to the coding sequence for the SGSH gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.N-acetyl-alpha-D-glucosaminidase (AAV.NAGLU) gene therapy vector comprises a vector genome comprising eight miR target sequences of the miR-183 cluster (including miR-182 target sequences, and miR-183 target sequences, or combinations thereof) operably linked to the coding sequence for the NAGLU gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.survival motor neuron 1 (AAV.SMN1) gene therapy vector comprises a vector genome comprising eight miR target sequences of the miR-183 cluster (including miR-182 target sequences, and miR-183 target sequences, or combinations thereof) operably linked to the coding sequence for the SMN1 gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.tripeptidyl peptidase 1 (AAV.TPP1) gene therapy vector comprises a vector genome comprising eight miR target sequences of the miR-183 cluster (including miR-182 target sequences, and miR-183 target sequences, or combinations thereof) operably linked to the coding sequence for the TPP1 gene. In certain embodiments, the vector genome comprises multiple copies of the same miR target sequence each separated by a spacer which may be the same or which may differ from each other. In another embodiment, the vector genome comprises multiple copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.anti-human epidermal growth factor receptor 2 antibody (AAV.anti-HER2) gene therapy vector comprises a vector genome comprising eight miR target sequences of the miR-183 cluster (including miR-182 target sequences target sequences, or combinations thereof) operably linked to the coding sequence for the anti-HER2 antibody. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.ornithine transcarbamylase (AAV.OTC) gene therapy vector comprises a vector genome comprising eight miR target sequences of the miR-183 cluster (including miR-182 target sequences, and miR-183 target sequences, or combinations thereof) operably linked to the coding sequence for the OTC gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.low-density lipoprotein receptor (AAV.LDLR) gene therapy vector comprises a vector genome comprising eight miR target sequences of the miRNA183 cluster (including miR-182 target sequences, and miR-183 target sequences, or combinations thereof) operably linked to the coding sequence for the LDLR gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.uridine diphosphate glucuronosyl transferase 1A1 (AAV.UGT1A1) gene therapy vector comprises a vector genome comprising eight miR target sequences of the miR-183 cluster (including miR-182 target sequences, and miR-183 target sequences, or combinations thereof) operably linked to the coding sequence for the UGT1A1 gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.anti-proprotein convertase subtilisin/kexin type 9 antibody (AAV.anti-PCSK9 Ab) gene therapy vector comprises a vector genome comprising aeight miR target sequences of the miR-183 cluster (including miR-182 target sequences, and miR-183 target sequences, or combinations thereof) operably linked to the coding sequence for the anti-PCSK9 Ab. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.Factor VIII (AAV.FVIII) gene therapy vector comprises a vector genome comprising eight target sequences of the miR-183 cluster (including miR-182 target sequences, and miR-183 target sequences, or combinations thereof) operably linked to the coding sequence for the FVIII gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.Factor IX (AAV.IX) gene therapy vector comprises a vector genome comprising eight miR target sequences of the miR-183 cluster (including miR-182 target sequences, and miR-183 target sequences, or combinations thereof) operably linked to the coding sequence for the FIX gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In certain embodiments, an AAV.myotubularin 1 (AAV.MTM1) gene therapy vector comprises a vector genome comprising eight target sequences of the miRNA183 cluster (including miR-182 target sequences, and miR-183 target sequences, or combinations thereof) operably linked to the coding sequence for the MTM1 gene. In certain embodiments, the vector genome comprises multiple copies of the same miR target sequence each separated by a spacer which may be the same or which may differ from each other. In another embodiment, the vector genome comprises three to six copies of a miR-183 cluster target sequence, optionally wherein one or more of the target sequences is at least about 80% to about 99% complementarity to a miR-183 cluster member. In another embodiment, the vector comprises one, two, three, or four copies of a miR-183 target sequence. Such a vector genome may optionally contain additional target sequences that correspond to members of the miR-183 cluster. In certain embodiments, the vector genome contains a single miR target sequence for a miR-183 cluster member. In certain embodiments, the vector genome contains two miR target sequences for miR-183 cluster members and optionally at least one spacer. In certain embodiments, the vector contains three miR target sequences for miR-183 cluster members and optionally at least two spacers. In certain embodiments, the vector genome contains two or more miR target sequences for the miR-183 cluster which differ in sequence from one another. In certain embodiments, the vector genomes described herein are carried by a non-AAV vector.

In one embodiment, the expression cassette is in a vector genome delivered in an amount of about 1 × 10⁹ GC per gram of brain mass to about 1 × 10¹³ genome copies (GC) per gram (g) of brain mass, including all integers or fractional amounts within the range and the endpoints. In another embodiment, the dosage is 1 × 10¹⁰ GC per gram of brain mass to about 1 × 10¹³ GC per gram of brain mass. In specific embodiments, the dose of the vector administered to a patient is at least about 1.0 × 10⁹ GC/g, about 1.5 × 10⁹ GC/g, about 2.0 × 10⁹ GC/g, about 2.5 × 10⁹ GC/g, about 3.0 × 10⁹ GC/g, about 3.5 × 10⁹ GC/g, about 4.0 × 10⁹ GC/g, about 4.5 × 10⁹ GC/g, about 5.0 × 10⁹ GC/g, about 5.5 × 10⁹ GC/g, about 6.0 × 10⁹ GC/g, about 6.5 × 10⁹ GC/g, about 7.0 × 10⁹ GC/g, about 7.5 × 10⁹ GC/g, about 8.0 × 10⁹ GC/g, about 8.5 × 10⁹ GC/g, about 9.0 × 10⁹ GC/g, about 9.5 × 10⁹ GC/g, about 1.0 × 10¹⁰ GC/g, about 1.5 × 10¹⁰ GC/g, about 2.0 × 10¹⁰ GC/g, about 2.5 × 10¹⁰ GC/g, about 3.0 × 10¹⁰ GC/g, about 3.5 × 10¹⁰ GC/g, about 4.0 × 10¹⁰ GC/g, about 4.5 × 10¹⁰ GC/g, about 5.0 × 10¹⁰ GC/g, about 5.5 × 10¹⁰ GC/g, about 6.0 × 10¹⁰ GC/g, about 6.5 × 10¹⁰ GC/g, about 7.0 × 10¹⁰ GC/g, about 7.5 × 10¹⁰ GC/g, about 8.0 × 10¹⁰ GC/g, about 8.5 × 10¹⁰ GC/g, about 9.0 × 10¹⁰ GC/g, about 9.5 × 10¹⁰ GC/g, about 1.0 × 10¹¹ GC/g, about 1.5 × 10¹¹ GC/g, about 2.0 × 10¹¹ GC/g, about 2.5 × 10¹¹ GC/g, about 3.0 × 10¹¹ GC/g, about 3.5 × 10¹¹ GC/g, about 4.0 × 10¹¹ GC/g, about 4.5 × 10¹¹ GC/g, about 5.0 × 10¹¹ GC/g, about 5.5 × 10¹¹ GC/g, about 6.0 × 10¹¹ GC/g, about 6.5 × 10¹¹ GC/g, about 7.0 × 10¹¹ GC/g, about 7.5 × 10¹¹ GC/g, about 8.0 × 10¹¹ GC/g, about 8.5 × 10¹¹ GC/g, about 9.0 × 10¹¹ GC/g, about 9.5 × 10¹¹ GC/g, about 1.0 × 10¹² GC/g, about 1.5 × 10¹² GC/g, about 2.0 × 10¹² GC/g, about 2.5 × 10¹² GC/g, about 3.0 × 10¹² GC/g, about 3.5 × 10¹² GC/g, about 4.0 × 10¹² GC/g, about 4.5 × 10¹² GC/g, about 5.0 × 10¹² GC/g, about 5.5 × 10¹² GC/g, about 6.0 × 10¹² GC/g, about 6.5 × 10¹² GC/g, about 7.0 × 10¹² GC/g, about 7.5 × 10¹² GC/g, about 8.0 × 10¹² GC/g, about 8.5 × 10¹² GC/g, about 9.0 × 10¹² GC/g, about 9.5 × 10¹² GC/g, about 1.0 × 10¹³ GC/g, about 1.5 × 10¹³ GC/g, about 2.0 × 10¹³ GC/g, about 2.5 × 10¹³ GC/g, about 3.0 × 10¹³ GC/g, about 3.5 × 10¹³ GC/g, about 4.0 × 10¹³ GC/g, about 4.5 × 10¹³ GC/g, about 5.0 × 10¹³ GC/g, about 5.5 × 10¹³ GC/g, about 6.0 × 10¹³ GC/g, about 6.5 × 10¹³ GC/g, about 7.0 × 10¹³ GC/g, about 7.5 × 10¹³ GC/g, about 8.0 × 10¹³ GC/g, about 8.5 × 10¹³ GC/g, about 9.0 × 10¹³ GC/g, about 9.5 × 10¹³ GC/g, or about 1.0 × 10¹⁴ GC/g brain mass.

In certain embodiments, the miR target sequence -containing compositions provided herein minimize the dose, duration, and/or amount of immunosuppressive co-therapy required by the patient. Currently, immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an antimetabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 In certain embodiments, the miR target sequence -containing compositions provided herein eliminate the need for immunosuppressive therapy prior to, during, or following delivery of a gene therapy (e.g., rAAV) vector.

In one embodiment, a composition comprising the expression cassette as described herein is administrated once to the subject in need. In certain embodiments, the expression cassette is delivered via an rAAV.

It should be understood that the description of the methods is intended to be applied to other compositions, regiments, aspects, embodiments and methods described across the Specification.

### 6. Kit

In certain embodiments, a kit is provided which includes a concentrated expression cassette (e.g., in a viral or non-viral vector) suspended in a formulation (optionally frozen), optional dilution buffer, and devices and components required for intrathecal, intracerebroventricular or intracisternal administration. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the description of the kits is intended to be applied to other compositions, regiments, aspects, embodiments and methods described across the Specification.

### 7. Device

In one aspect, the compositions provided herein may be administered intrathecally via the method and/or the device described, e.g., in WO 2017/136500, which is incorporated herein by reference in its entirety. Alternatively, other devices and methods may be selected. In summary, the method comprises the steps of advancing a spinal needle into the cisterna magna of a patient, connecting a length of flexible tubing to a proximal hub of the spinal needle and an output port of a valve to a proximal end of the flexible tubing, and after said advancing and connecting steps and after permitting the tubing to be self-primed with the patient’s cerebrospinal fluid, connecting a first vessel containing an amount of isotonic solution to a flush inlet port of the valve and thereafter connecting a second vessel containing an amount of a pharmaceutical composition to a vector inlet port of the valve. After connecting the first and second vessels to the valve, a path for fluid flow is opened between the vector inlet port and the outlet port of the valve and the pharmaceutical composition is injected into the patient through the spinal needle, and after injecting the pharmaceutical composition, a path for fluid flow is opened through the flush inlet port and the outlet port of the valve and the isotonic solution is injected into the spinal needle to flush the pharmaceutical composition into the patient. This method and this device may each optionally be used for intrathecal delivery of the compositions provided herein. Alternatively, other methods and devices may be used for such intrathecal delivery.

It should be understood that the description of the devices is intended to be applied to other compositions, regiments, aspects, embodiments and methods described across the Specification.

### EXAMPLES

The invention is now described with reference to the following examples. These examples are provided for the purpose of illustration only and the invention should in no way be construed as being limited to these examples but rather should be construed to encompass any and all variations that become evident as a result of the teachings provided herein.

### Example 1: Methods

### Animals

All animal procedures were approved by the Institutional Animal Care and Use Committee of the University of Pennsylvania. Rhesus macaques (Macaca mulatta) were procured from Covance Research Products, Inc. (Alice, TX) and Primgen/Prelabs Primates (Hines, IL). Animals were housed in an Association for Assessment and Accreditation of Laboratory Animal Care (AAALAC) International-accredited Nonhuman Primate Research Program facility at the University of Pennsylvania in stainless steel squeeze back cages. Animals received varied enrichments such as food treats, visual and auditory stimuli, manipulatives, and social interactions.

C56BL/6J mice (stock #000664) were purchased from the Jackson Laboratory. Animals were housed in an AAALAC International-accredited mouse barrier vivarium at the Gene Therapy Program, University of Pennsylvania, in standard caging of 2 to 5 animals per cage with enrichment (Nestlets nesting material). Cages, water bottles, and bedding substrates were autoclaved in the barrier facility and cages were changed once per week. An automatically controlled 12-hour light/dark cycle was maintained. Each dark period began at 1,900 hours (± 30 minutes). Irradiated laboratory rodent food was provided ad libitum.

### Vectors

The AAV9.PHP.B trans plasmid (pAAV2/PHP.B) was generated with a QuikChange Lightning Site-Directed Mutagenesis Kit (Agilent Technologies, Santa Clara, CA, Cat #210515) using pAAV2/9 (Penn Vector Core) as the template, following the manufacturer’s manual. pAAV2/9 and pAAV2/hu68 were provided by the Penn Vector Core. AAV vectors were produced and titrated by the Penn Vector Core (as described previously by Lock, M., et al. Hum Gene Ther 21:1259-1271, 2010). Briefly, HEK293 cells were triple-transfected and the culture supernatant was harvested, concentrated, and purified with an iodixanol gradient. The purified vectors were titrated with droplet digital PCR using primers targeting the rabbit beta-globin polyA sequence (as previously by Lock, M., e al. Hum Gene Ther Methods 25:115-125, 2014). The human alpha -L-iduronidase (hIDUA) sequence was obtained through back-translation and codon-optimization of the hIDUA isoform a precursor protein sequence NP_0.0194.2 and was cloned under the CB7 promoter. Dorsal root ganglion (DRG)-enriched microRNA sequences were selected from the public database available at mirbase.org. Four tandem repeats of the target for the DRG-enriched miR were cloned in the 3′ untranslated region (UTR) of green fluorescent protein (GFP) or hIDUA cis plasmids.

### In Vivo Studies

Mice received 1×10¹² genome copies (GCs; 5×10¹³ GC/kg) of AAV-PHP.B, or 4×10¹² GCs (2×10¹⁴ GC/kg) of AAV9 vectors encoding enhanced GFP with or without miR targets in 0.1 mL via the lateral tail vein and were euthanized by inhalation of CO₂ 21 days post injection. Tissues were promptly collected, starting with brain, and immersion-fixed in 10% neutral buffered formalin for about 24 h, washed briefly in phosphate buffered saline (PBS), and equilibrated sequentially in 15% and 30% sucrose in PBS at 4° C. Tissues were then frozen in optimum cutting temperature embedding medium and cryosectioned for direct GFP visualization (brain were sectioned at 30 µm, and other tissues at 8 µm thickness). Images were acquired with a Nikon Eclipse Ti-E fluorescence microscope. GFP expression in DRGs was analyzed by immunohistochemistry (IHC). Spinal columns with DRGs were fixed in formalin for 24 h, decalcified in 10% ethylenediaminetetraacetic acid (pH 7.5) until soft, and paraffin embedded following standard protocols. Sections were deparaffinized through an ethanol and xylene series, boiled for 6 min in 10 mM citrate buffer (pH 6.0) to perform antigen retrieval, blocked sequentially with 2% H₂O₂ (15 min), avidin/biotin blocking reagents (15 min each; Vector Laboratories, Burlingame, CA), and blocking buffer (1% donkey serum in PBS + 0.2% Triton for 10 min) followed by incubation with primary (1 h at 37° C.) and biotinylated secondary antibodies (diluted 1:500, 45 min; Jackson ImmunoResearch, West Grove, PA) diluted in blocking buffer. As rabbit antibody against GFP was used as the primary antibody (NB600-308, Novus Biologicals, Centennial, CO; diluted 1:500). A Vectastain Elite ABC kit (Vector Laboratories, Burlingame, CA) with DAB as substrate allowed us to visualize bound antibodies as brown precipitate.

Non-human primates (NHP) received 3.5 × 10¹³ GCs of AAVhu68.GFP vectors or 1 × 10¹³ GCs of AAVhu68.hIDUA vectors in a total volume of 1 mL injected into the cisterna magna, under fluoroscopic guidance (as previously described by Katz, N., et al. Hum Gene Ther Methods 29:212-219, 2018). Period blood collection and cerebrospinal fluid (CSF) taps were performed for safety readouts. Serum chemistry, hematology, coagulation, and CSF analyses were performed by the contract facility Antech Diagnostics (Morrisville, NC). Animals were euthanized with intravenous pentobarbital overdose and necropsied; the tissues were then harvested for comprehensive histopathologic examination. Collected tissues were immediately fixed in formalin and paraffin embedded. For histopathology, tissue sections were stained with hematoxylin and eosin following standard protocols. IHC for GFP expression was carried out as described for the mouse studies but using a different antibody against GFP (goat antibody NB100-1770, Novus Biologicals; diluted 1:500, incubated overnight at 4° C.). Immunostaining for hIDUA was performed using a sheep antibody against hIDUA (AF4119, R&D Systems, Minneapolis, MN; diluted 1:200) following the above protocol for IHC. In addition, sections were stained for hIDUA by immunofluorescence (IF) using the same primary antibody. For IF, sections were deparaffinized and treated for antigen retrieval as described above, and then blocked with 1% donkey serum in PBS + 0.2% Triton for 15 min followed by sequential incubation with primary (2 h at room temperature, diluted 1:50) and FITC-labeled secondary (45 min; Jackson ImmunoResearch; diluted 1:100) antibodies diluted in blocking buffer. Sections were mounted in Fluoromount G with DAPI as a nuclear counterstain.

In situ hybridization (ISH) was performed using probes specific for the codon-optimized RNA transcribed from the vector genome that do not bind to endogenous monkey IDUA RNA. Z-shaped probe pairs were synthesized by Life Technologies (Carlsbad, CA) and ISH was performed on paraffin sections using the Life Technologies ViewRNA ISH Tissue Assay kit according to the manufacturer’s protocol. The deposition of Fast Red precipitates indicating positive signals was imaged by fluorescence microscopy using a rhodamine filter set. Tissue sections with IDUA IHC were scanned for quantification purposes using an Aperio Versa slide scanner (Leica Biosystems, Buffalo Grove, IL).

### Histopathology and Morphometry

A board-certified veterinary pathologist who was blinded to the vector group established severity grades defined with 0 as absence of lesion, 1 as minimal (<10%), 2 mild (10-25%), 3 moderate (25-50%), 4 marked (50-95%), and 5 severe (>95%). Dorsal axonopathy scores were established in each animal from at least 3 cervical, 3 thoracic, and 3 lumbar sections; the DRG severity grades were established from at least 3 cervical, 3 thoracic, and 3 lumbar segments; and the median nerve score was the sum of axonopathy and fibrosis severity grades with a maximal possible score of 10 and was established on the distal and proximal portions of left and right nerves. For quantification of transgene expression, a board-certified Veterinary Pathologist counted cells immunostained with anti-GFP or anti-hIDUA antibodies by comparing with signal from control slides obtained from untreated animals. The total number of positive cells per x20 magnification field was counted using the ImageJ cell counter tool on a minimum of five fields per structure and per animal.

### Vector Biodistribution

NHP tissue DNA was extracted with a QIAamp DNA Mini Kit (Qiagen, Germany, Cat #51306) and vector genomes were quantified by real-time PCR using Taqman reagents (Applied Biosystems, Life Technologies, Foster City, CA) and primers/probes targeting the rBG polyadenylation sequence of the vectors.

### Immunology

Peripheral blood T-cell responses against hIDUA were measured by interferon gamma enzyme-linked immunosorbent spot assays according to previously published methods (Gao et al., 2009), using peptide libraries specific for the hIDUA transgene. Positive response criteria were >55 spot forming units per 10⁶ lymphocytes and three times the medium negative control upon no stimulation. In addition, T-cell responses were assayed in lymphocytes that were extracted from spleen, liver, and deep cervical lymph nodes after necropsy on study day 90. Antibodies to hIDUA were measured in serum (1:1,000 sample dilution) (as previously described by Hinderer, C., et al. Mol Ther 23:1298-1307, 2015).

Cytokine/Chemokine analysis: CSF samples were collected and stored at -80C until the time of analysis. CSF samples were analyzed using a Milliplex MAP kit containing the following analytes: sCD137, Eotaxin, sFasL, FGF-2, Fractalkine, Granzyme A, Granzyme B, IL-1α, IL-2, IL-4, IL-6, IL-16, IL-17A, IL-17E/IL-25, IL-21, IL-22, IL-23, IL-28A, IL-31, IL-33, IP-10, MIP-3α, Perforin, TNFβ. CSF samples were evaluated in duplicate and analyzed in a FLEXMAP 3D instrument using Luminex® xPONENT® 4.2; Bio-Plex Manager™ Software 6.1. Only samples with a %CV of less than 20% were included in the analysis.

### In Vitro Studies

The miR-183 human microRNA expression plasmid was modified from Origene MI0000273 vector by deleting the KpnI-PstI fragment encoding GFP and partial internal ribosome entry sites. We confirmed the lack of GFP expression from the modified vector by transient transfection and anti-GFP immunoblotting. We performed polyethylenimine-mediated transient transfection in HEK293 cells with GFP cis-plasmids harboring microRNA binding sites located in the 3′-UTR of the GFP expression cassette. At 72 hours post-transfection, we lysed the cells in 50 mM Tris-HCl, pH 8.0, 150 mM NaCl, and 0.5% Triton X-100 with protease inhibitors. A total of 13 µg of cell lysates was used for anti-GFP immunoblotting followed by electrochemiluminescence-based signal detection and quantification. We performed triplicate experiments for statistical analysis.

### Statistical Analysis

Statistical differences between groups were assessed using the Wilcoxon rank sum test.

### Example 2: microRNA Mediated Inhibition of Transgene Expression Reduces Dorsal Root Ganglia Toxicity by AAV

Delivering adeno-associated virus (AAV) vectors into the CNS of non-human primates (NHP) via the blood or cerebral spinal fluid is associated with dorsal root ganglia (DRG) toxicity. This may be caused by high rates of transduction, which can cause endoplasmic reticulum stress from overproduction of the transgene product. We developed an approach to eliminate toxicity associated with CNS-directed AAV gene therapy by introducing miRNA target sequences into the vector genome within the 3′ untranslated region of the corresponding transgene mRNA. The expression cassette for ITR.CB7.CI.eGFP.miR-145(four copies).rabbit beta globin, 3′ITR is provided in SEQ ID NO: 10, the expression cassette for ITR.CB7.CI.GFP.miR-182(four copies).rabbit beta globin, 3′ITR is provided in SEQ ID NO: 11, the expression cassette for ITR.CB7.CI.GFP.miR-96(four copies).rabbit beta globin, 3′ITR is provided in SEQ ID NO: 12, and the expression cassette for ITR.CB7.CI.GFP.miR-183(four copies).rabbit beta globin, 3′ITR is provided in SEQ ID NO: 13.

### AAV Vectors Cause DRG Degeneration in NHPs

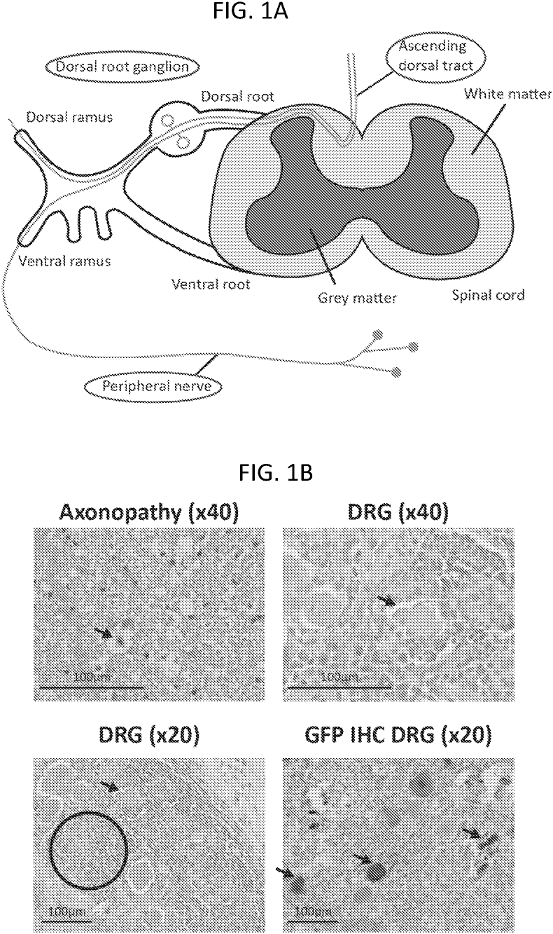

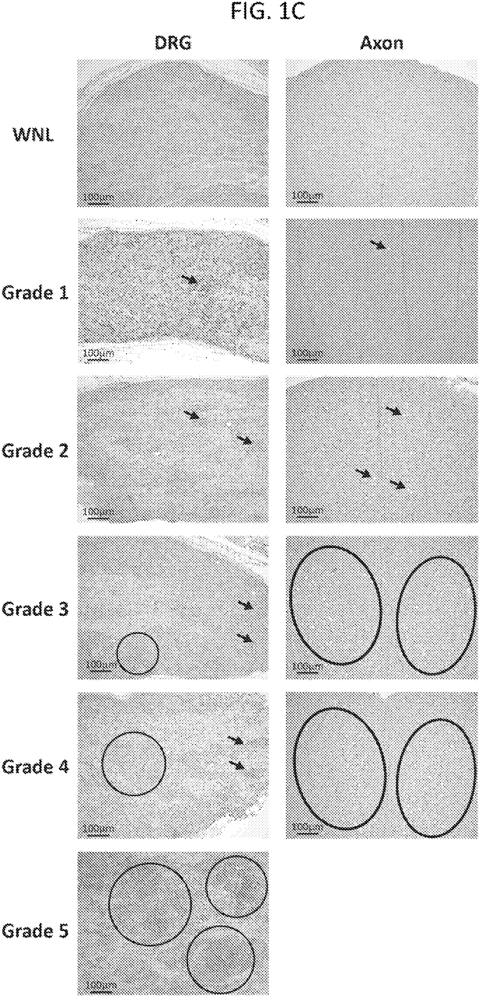

Based on our experience in DRG toxicity in NHPs, we developed a system to quantify the severity of toxicity. We evaluated cell bodies located along the spinal cord in the DRGs, the axons within the peripheral nerves, and the axons that ascend the dorsal white-matter tracts (FIG. 1B). We believe the primary lesion is degeneration of the sensory neuron cell body located in DRG. The lesion is histologically characterized by hypereosinophilia, irregular cell shapes, disruption of Nissl substance (central chromatolysis), and loss of nuclear boundaries along with mononuclear cell infiltration (FIG. 1B). Cells expressing high levels of transgene protein are more likely to undergo degeneration as evidenced by transgene product immunostaining in animals that received an ICM administration of an AAV vector expressing green fluorescent protein (GFP; FIG. 1B). Secondary to the cell body death is axonopathy, which is degeneration of the distal and proximal axons. Axonopathy is characterized by missing axons, dilated myelin sheaths surrounding cell debris, and macrophages (FIG. 1B). FIG. 1C illustrates examples of different levels of DRG toxicity and spinal cord axonopathy. The grades are based on the proportion of affected tissue at high-power field histopathologic examination: 1 minimal (<10%), 2 mild (10-25%), 3 moderate (25-50%), 4 marked (50-95%) and 5 severe (>95%).

Our total experience of adolescent/adult NHPs administered AAV vectors into the CSF via ICM or lumbar puncture (LP) route totals 219 monkeys spanning 27 studies that encompasses previous published toxicology studies (Hordeaux, J., et al. Mol Ther Methods Clin Dev 10:68-78, 2018; Hordeaux, J., et al. Mol Ther Methods Clin Dev 10:79-88, 2018) and the NHP experiments described in the Examples below as well as in a number of unpublished studies. This experience includes five capsids, 20 transgenes, five promoters (CAG, CB7, UBC, hSyn, and MeP426), doses from 1 × 10¹² GC to 3 × 10¹⁴ GC, vector purified by gradients or columns, three formulations (phosphate buffered saline and two different artificial CSF), and rhesus and cynomologus macaques at various developmental stages. In every experimental group, we observed DRG toxicity and axonopathy. The pathology peaks about one month after injection and does not progress for up to six months, which is the longest period evaluated in mature macaques. In most cases, the pathology is mild to moderate. However, high doses of vectors expressing GFP injected ICM can lead to severe pathology associated with ataxia.

### miRNAs Specifically Expressed in DRG Neurons Can Ablate AAV Transgene Expression

Several mechanisms were evaluated when considering ways to mitigate DRG toxicity. In previous studies, we analyzed the role of destructive adaptive immune responses to the transduced DRGs by immune suppressing NHPs that were administered ICM AAV9 vectors expressing human IDUA or human IDS. Treatment with mycophenolate mofetil (MMF) and rapamycin blunted the adaptive immune response to the vector and transgene product but did not significantly impact DRG toxicity and axonopathy (Hordeaux, J., et al. Mol Ther Methods Clin Dev 10:68-78, 2018; Hordeaux, J., et al. Mol Ther Methods Clin Dev 10:79-88, 2018).

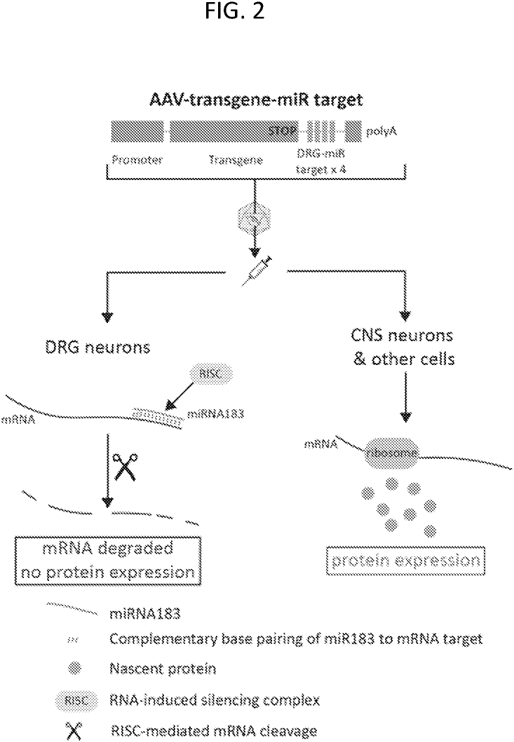

One possibility that has not been previously investigated is whether overexpression of transgene products in highly transduced DRGs is the cause neuronal injury and degeneration of the cell body and associated axons followed by a reactive inflammatory response (FIG. 2A). Accordingly, to specifically ablate transgene expression in DRG we cloned miRNA targets that are solely expressed in DRG neurons into the 3′ untranslated regions of the transgene (FIG. 2B). Any mRNA expressed from the vector would be destroyed by the endogenously expressed miRNAs.

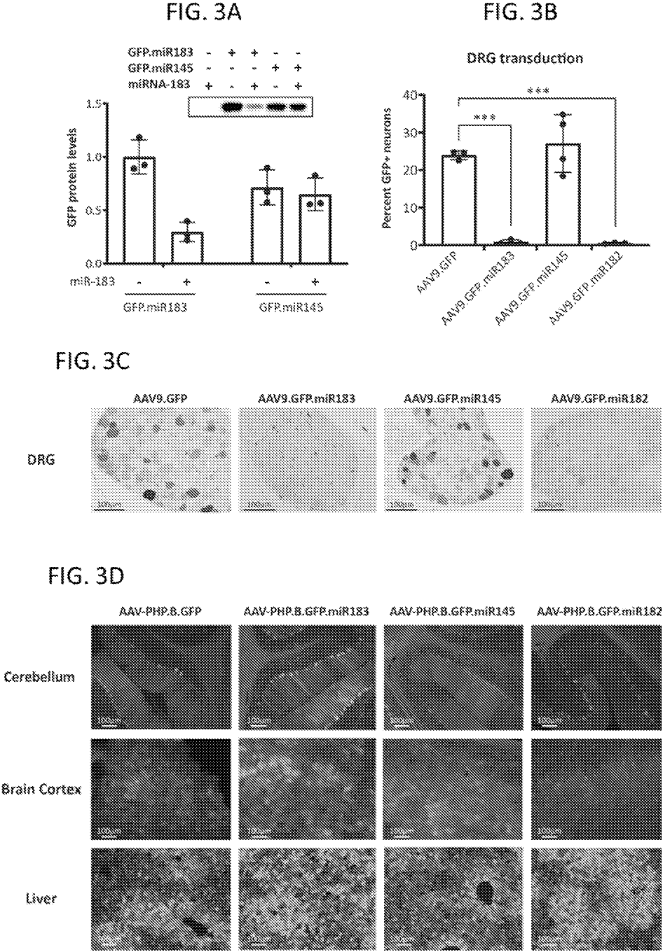

We used an in vitro assay to evaluate the activity and specificity of the miRNA strategy. We constructed AAV cis plasmids to include four repeat concatemers of the target miRNA sequences in the 3′ untranslated region of the expression cassette (FIG. 2B). AAV cis plasmids were co-transfected into HEK293 cells with plasmids expressing miR-183. Expression of the transgene GFP was reduced in the presence of miR-183 only when it contained the cognate recognition sequence (FIG. 3A).

The in vivo activity and specificity of potential miRNA targets within AAV vectors was screened in C57Bl/6J mice. We evaluated GFP-expressing vectors with or without miRNA targets from two members of the miRNA-183 complex (miR-182 and miR-183) as well as miR-145. We initially tested miR-96, another member of the miR-183 complex, but eliminated it due to decreased transgene expression in mice cortices (not shown). Animals received high-dose intravenous (IV) injections of AAV9 to target DRGs and high-dose AAV-PHP.B (AAV9-PHP.B.CB7.CI.GFP.rBG) injections to target the CNS. Animals were necropsied on day 21 and analyzed for GFP expression in DRGs by immunohistochemistry (IHC) and direct-fluorescence microscopy in brain and liver. Expression of GFP in DRG neurons was substantially reduced with vectors containing miR-183 and miR-182 targets, however miR-145 targets had no effect (FIG. 3B and FIG. 3C). There was no apparent reduction of expression in liver or other CNS compartments with vectors containing any of the miR targets. Expression seemed to be slightly enhanced in CNS with the miR-183 vector (FIG. 3D). In this mouse experiment, we were unable to assess the impact of miR-183 transgene suppression on pathology since the vector-induced DRG toxicity has only been observed in NHPs.

### Restricted Transgene Expression by miR-183 Reduces DRG Toxicity in NHPs

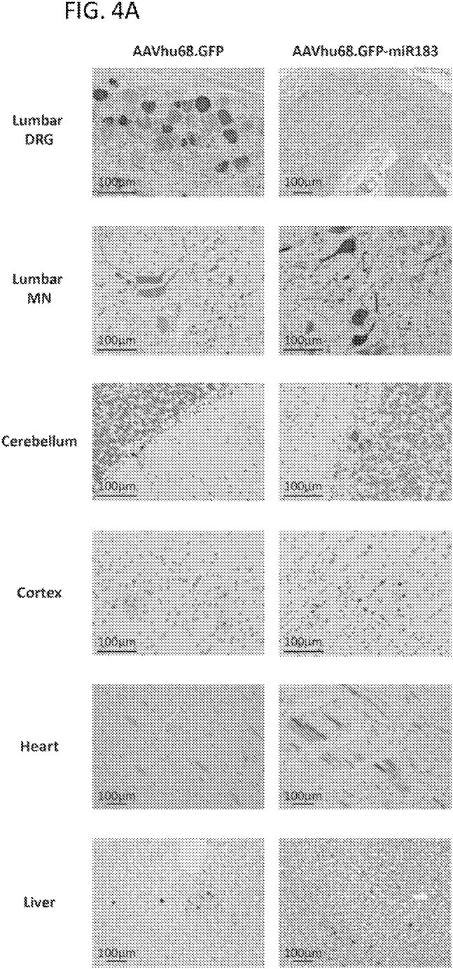

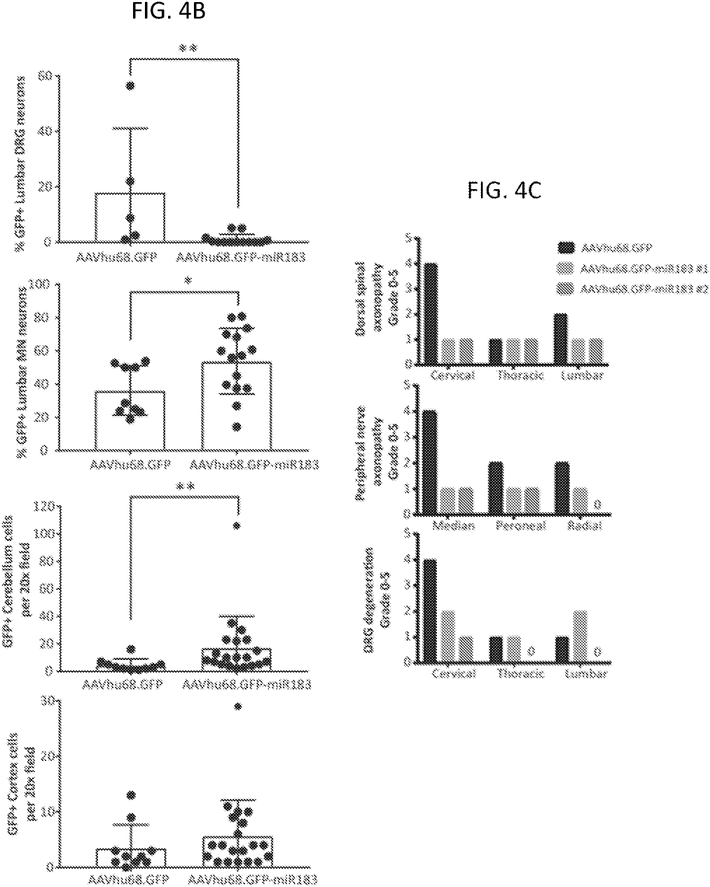

Based on the encouraging data in mice, we evaluated the GFP miR-183 expression cassette in NHPs. We ICM injected AAVhu68 vectors expressing GFP (AAV9.CB7.CI.GFP.rBG) (N=2) or GFP miR-183 (AAV9.CB7.CI.GFP.miR-183.rBG) (N=4) from a CB7 promoter in rhesus macaques (3.5 × 10¹³ GC). Half of the animals were necropsied on day 14 for GFP expression (FIG. 4B - representative IHC for GFP expression; FIG. 4B - quantitation of expression). The remaining animals were necropsied on day 60 to evaluate expression and DRG toxicity (FIG. 4C - DRG degeneration, dorsal spinal axonopathy, and peripheral nerve axonopathy). Animals tolerated the ICM-administered vector without clinical sequalae. There was a statistically significant reduction of GFP expression in DRG with the miR-183 vector and enhancement or similar expression elsewhere including lumbar motor neurons, cerebellum, cortex, heart, and liver (FIG. 4A and FIG. 4B; Table 1). This was associated with a remarkable reduction of pathology across nine regions (DRG and dorsal spinal axonopathy at cervical, thoracic and lumbar spine and axonopathy of median, peroneal and radial nerves; FIG. 4C). Without miR-183 targets in the vector, pathology was present in all regions and evenly distributed between grade 4, grade 2, and grade 1. With the miR-183 vector, the greatest pathology was grade 2 and was present in only 11% of regions; the remaining regions were either grade 1 (72%) or no pathology (16%).

These studies demonstrated that GFP expression is selectively repressed in DRG sensory neurons with vectors that contain miR-183 targets. Other CNS neurons and peripheral organs were not affected. Accordingly, DRG toxicity and secondary axonopathy were reduced from marked/severe to minimal levels in the context of a highly immunogenic/toxic transgene (GFP).

### Example 3: Specific Repression of Therapeutic Protein Expression in DRG Following Delivery Via AAV With a Vector Genome Having miRNA Target Sequences

We further evaluated miR-183 target sequences in NHPs using vectors that expressed human IDUA—an enzyme deficient in patients with mucopolysaccharidosis I. Studies with this human transgene were the first to highlight DRG toxicity in NHPs (Hordeaux, J., et al. Mol Ther Methods Clin Dev 10:79-88, 2018). The experiment included three groups (N=3/group): 1) group 1 - control vector alone without miR-183 targets (AAVhu68.CB7.CI.hIDUAcoV1.rBG); 2) group 2 - control vector without miR-183 targets (AAVhu68.CB7.CI.hIDUAcoV1.rBG) in animals treated with steroids (prednisolone 1 mg/kg/day from day minus 7 to day 30 followed by progressive taper off); and 3) group 3 -vector with miR-183 targets (AAVhu68.CB7.CI.hIDUAcoV1.miR-183.rBG). All vector genomes included an hIDUA coding sequence under the control of a chicken β-actin promoter and CMV enhancer elements (referred to as the CB7 promoter), a chimeric intron (CI) consisting of a chicken β-actin splice donor (973 bp, GenBank: X00182.1) and a rabbit β-globin splice acceptor element, and a rabbit β-globin polyadenylation signal (rBG, 127 bp, GenBank: V00882.1). The vector genome for ITR.CB7.CI.hIDUAcoV1.rBG.ITR is provided in SEQ ID NO: 14. The vector genome for ITR.CB7.CI.hIDUAcoV1.miR-183.rBG.ITR is provided in SEQ ID NO: 15. All animals received an ICM injection of an AAVhu68 vector (3.5 × 10¹³ GC) expressing hIDUA from the constitutive promoter CB7. Half of the animals were necropsied on day 14 for GFP expression. We conducted necropsies at day 90 to evaluate transgene expression (individual data points in Table 1) and DRG-related toxicity (individual data points in Table 2).

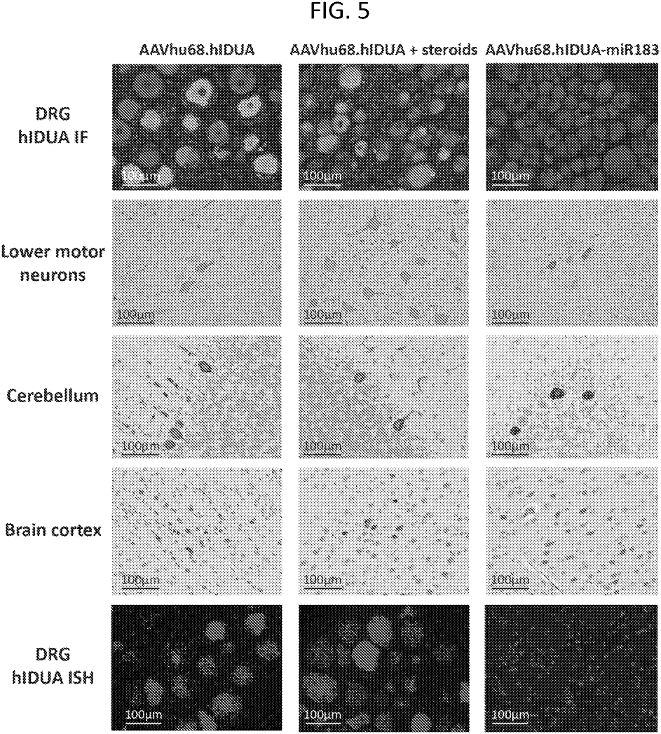

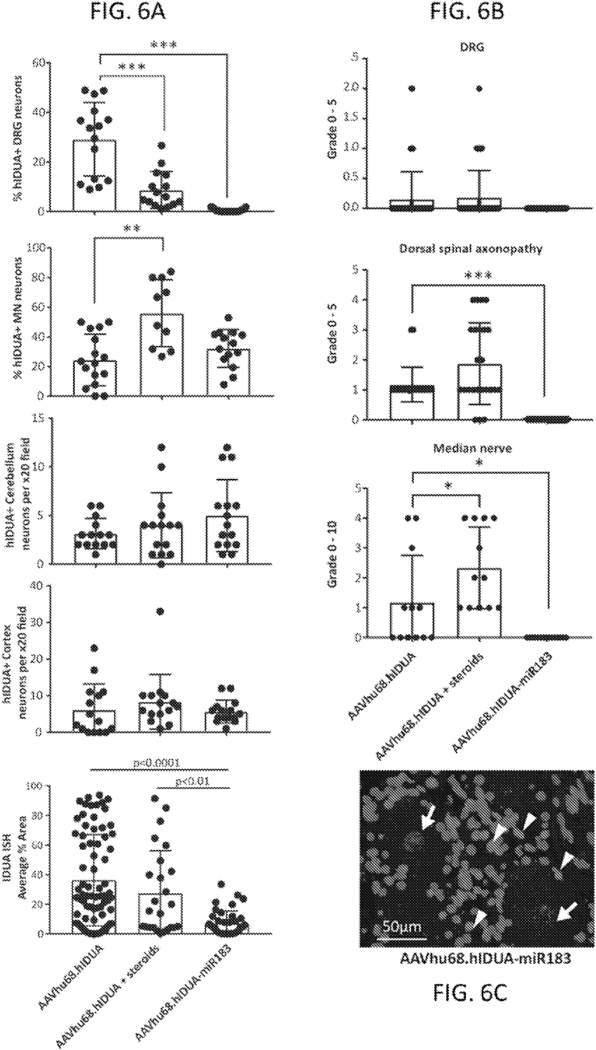

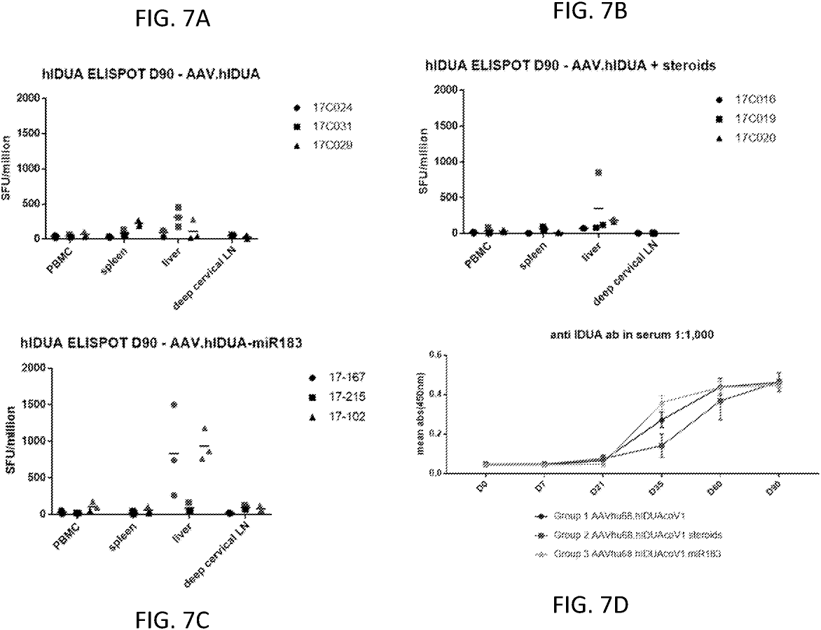

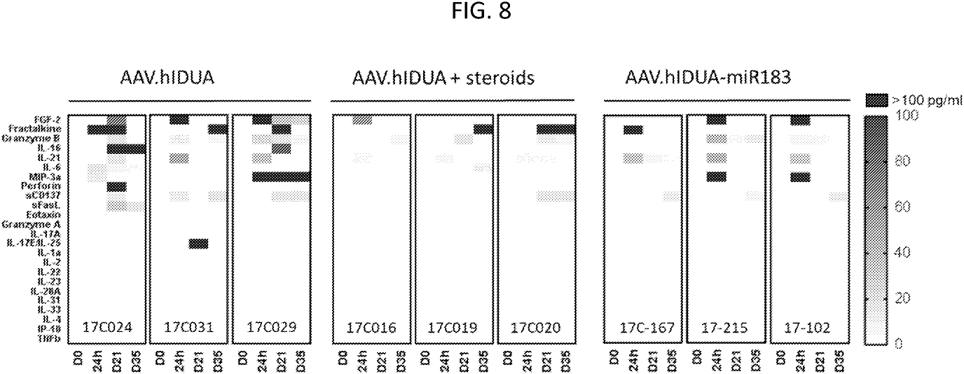

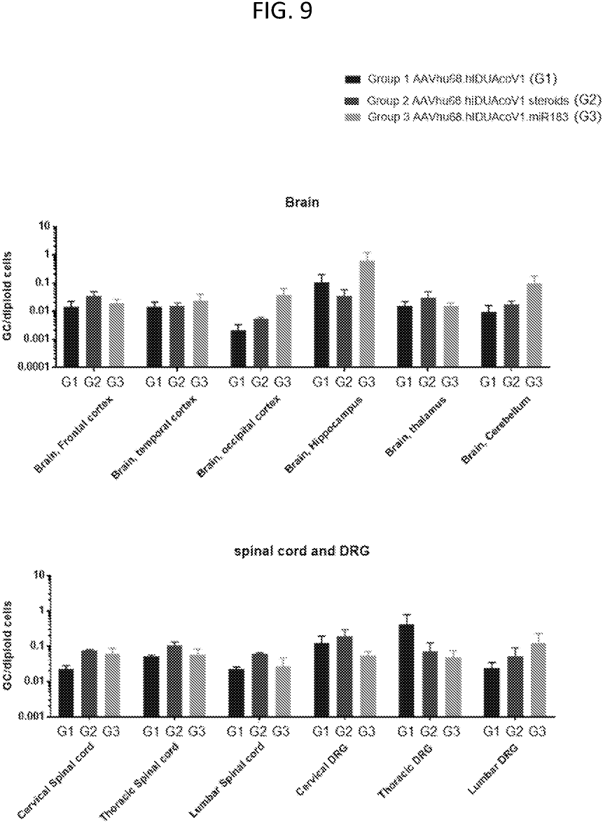

Animals from all groups tolerated ICM vector with no vector-related clinical findings or abnormalities in clinical pathology (Tables 3 and 4). Pleocytosis in CSF was very low and limited to one animal in group 2 and one animal in group 3 (Table 5). Both T-cell responses (measured by ELISPOT) and antibodies to hIDUA were detected in all three groups (FIGS. 7A - 7D). CSF cytokines were reduced in group 3 compared to group 1 at 21 and 35 days post-injection while levels were reduced in group 2 (steroids) at 24 hours (FIG. 8). Day 21-35 corresponds to peak expression of transgene when overexpression induced stress would be expected. Using immunofluorescence and in situ hybridization (ISH), we observed high expression of hIDUA in DRG in groups 1 and 2, which used the control vector (without miR-183 target; FIG. 5 and FIG. 6A). We detected low to moderate levels of hIDUA expression in other CNS compartments including lower motor neurons of the spinal cord and cerebellum and cortical neurons (FIG. 5 and FIG. 6A). Incorporating the miR-183 target into the vector (i.e., AAVhu68.hIDUA-mir-183/group 3) ablated hIDUA expression in DRG neurons without decreasing expression in the CNS(i.e., spinal cord, cerebellum, and cortex) as highlighted by immunofluorescence and immunohistochemistry (FIG. 5 and FIG. 6A). At the mRNA level (FIG. 5), cytoplasmic ISH signal in transduced neurons was decreased from 42% of area in animals dosed with AAVhu68.hIDUA to 7% in animals dosed with AAVhu68.hIDUA-miR-183 (FIG. 6A), which represents an 83% reduction. Reduction of hIDUA expression in DRGs by miR-183 was not due to decreased gene transfer since the biodistribution of vector throughout the CNS and DRGs was essentially the same across all groups (FIG. 9). Steroids moderately decreased expression in DRGs (p=0.0001) and increased it in lower motor neurons compared with group 1 (FIG. 5 and FIG. 6A). As expected, administration with the control vector (group 1) resulted in DRGs, dorsal column, and peripheral nerve pathology that was comparatively milder than with GFP-expressing vectors. However, pathology was completely absent in the DRG (p=0.0583), dorsal column (p<0.0001) and peripheral (median) nerve (p=0.0137) of animals transduced with miR-183 target-containing vector (group 3, FIG. 6B). Co-treatment with steroids (group 2) did not reduce toxicity of the parent vector (i.e. not containing miR-183 targets) (FIG. 6B) but was instead associated with a trend of worsening toxicity in the peripheral nerves (p=0.0256) and dorsal column (p=0.066).

TABLE 1

Individual transgene expression counts for NHP

| NHP id | Vector | % transgene + DRG neurons | % transgene + motor neurons | transgene + cells per high power field, cerebellum | Transgene + cells per high power field, cerebral cortex |
|---|---|---|---|---|---|
| RA2309 | AAVhu68.CB7.CI.eGFP.rBG | 0.9; 22; 8.7 | 28.6; 23.8; 18.75; 23.1; 25 | 7; 3; 1; 5; 2 | 9; 13; 3; 2; 1 |
| RQ9475 | AAVhu68.CB7.CI.eGFP.rBG | 56.5; 2.4 | 52.6; 50; 53.8; 50 | 2; 5; 1; 3; 16 | 3; 2; 0; 1; 1 |
| RA2102 | AAVhu68.CB7.CI.eGFP.miR183.rBG | 0; 1.6; 0.7 | 26.9; 55.9 | 8; 5; 2; 5; 10 | 4; 29; 10; 9; 2 |
| RA1652 | AAVhu68.CB7.CI.eGFP.miR183.rBG | 0.3; 0; 0; 5 | 57.1; 73.7; 70; 37.5 | 10; 4; 7; 10; 4 | 10; 6; 8; 4; 11 |
| RA2141 | AAVhu68.CB7.CI.eGFP.miR183.rBG | 5.1; 0; 0; 0 | 80.8; 60.7; 80; 37.5; 39.5 | 22; 15; 7; 3; 30 | 1; 1; 4; 4; 1 |
| RA2027 | AAVhu68.CB7.CI.eGFP.miR183.rBG | 0; 0 | 60; 68.2; 14.3; 45 | 13; 23; 35; 106; 23 | 3; 1; 2; 1; 3 |
| 17C024 | AAVhu68.CB7.CI.hIDUAcoV1.rBG | 48.7; 25.2; 47.4; 40.6; 36.7 | 26.1; 45.7; 23.5; 7.7; 14.3; 0 | 2; 3; 2; 4 | 8; 5; 17; 0; 0 |
| 17C031 | AAVhu68.CB7.CI.hIDUAcoV1.rBG | 34.5; 29.6; 49; 37; 33.7 | 28.6; 38.1; 19; 22.2; 50; 50 | 3; 2; 2; 3; 6 | 0; 11; 1; 1; 0 |
| 17C016 | AAVhu68.CB7.CI.MDUAcoV1.rBG + steroids | 7.8; 10.1; 3.9; 10.2; 1.8 | 47.6; 43.5 | 1; 0; 1; 2; 4 | 7; 8; 10; 10; 8 |
| 17C019 | AAVhu68.CB7.CI.hIDUAcoV1.rBG + steroids | 14.8; 26.6; 15.7; 19.5; 1.1; 33 | 26.7; 31.8 | 2; 4; 4; 12; 1 | 6; 1; 2; 3; 5 |
| 17C029 | AAVhu68.CB7.CI.hIDUAcoV1.rBG | 12.4; 10.9; 13.1; 9.8; 8.9 | 15; 4.8; 46.7; 0 | 5; 6; 2; 3; 1 | 3; 2; 11; 10; 23 |
| 17C020 | AAVhu68:CB7:CI.hIDUAcoV1.rBG + steroids | 2.5; 6; 4; 2.9; 4.7 | 80; 70; 84; 80; 66.7; 30 | 5; 4; 10; 4; 6 | 11; 6; 5; 10; 33 |
| 17-167 | AAVhu68.CB7.CI.hIDUAcoV1.miR183.rBG | 1.6; 0.5; 0; 0; 0.4 | 25; 19.4; 42.9; 7.7 | 5; 4; 2; 6; 3 | 12; 4; 6; 3; 6 |
| 17-215 | AAVhu68.CB7.CI.hIDUAcoV1.miR183.rBG | 0; 0; 0; 0; 0 | 42.9; 41.7; 12.5; 29.4; 52.9; 35.7 | 1; 1; 2; 6; 6 | 4; 4; 1; 5; 3 |
| 17-102 | AAVhu68.CB7.CI.hIDUAcoV1.miR183.rBG | 0.8; 0.4; 1.9; 1.8; 0 | 39.3; 41.2; 31.8; 28 | 12; 11; 3; 2; 11 | 7; 4; 12; 8; 6 |

TABLE 2

Individual DRG pathology, spinal cord dorsal columns axonopathy, and peripheral nerve axonopathy and fibrosis severity grades

| NHP id | Vector | DRG | Dorsal spinal cord | Peripheral nerves |
|---|---|---|---|---|
| RA2309 | AAVhu68.CB7.CI.eGFP.rBG | 4; 1; 1 | 4; 1; 2 | Median; 4 Peroneal; 2 Radial; 2 |
| RQ9475 | AA Vhu68.CB7.CI.eGFP.rBG | N/A (D14) | N/A (D14) | N/A (D14) |
| RA2102 | AAVhu68.CB7.CI.eGFP.miR183.rBG | 1; 0; 0 | 1; 1; 1 | Median; 1 Peroneal; 1 Radial; 0 |
| RA1652 | AAVhu68.CB7.CI.eGFP.miR183.rBG | 2; 1; 2 | 1; 1; 1 | Median; 1 Peroneal; 1 Radial; 1 |
| RA2141 | AAVhu68.CB7.CI.eGFP.miR183.rBG | N/A (D14) | N/A (D14) | N/A (D14) |
| RA2027 | AAVhu68.CB7.CI.eGFP.miR183.rBG | N/A (D14) | N/A (D14) | N/A (D14) |
| 17C024 | AAVhu68.CB7.CI.hIDUAcoV1.rBG | 2; 2; 0; 0; 0; 0; 0; 0; 0; 0; 0; 0; 0; 0 | 3; 3; 1; 1; 1; 1; 1; 1 | Median; 4; 4; 3; 1 |
| 17C031 | AAVhu68.CB7.CI.hIDUAcoV1.rBG | 1; 1; 1; 0; 0; 0; 0; 0; 0; 0; 0; 0; 0; 0; 0; 0 | 1; 1; 1; 1; 1; 1; 1; 1 | Median; 1; 1; 0; 0 |
| 17C016 | AAVhu68.CB7.CI.hIDUAcoV1.rBG + steroids | 0; 0; 0; 0; 0; 0; 0; 0; 0; 0; 0 | 1; 1; 1; 0; 0; 0; 1; 1 | Median; 1; 1; 1; 1 |
| 17C019 | AAVhu68.CB7.CI.hIDUAcoV1.rBG + steroids | 2; 1; 0; 0; 0; 0; 0; 0; 0; 0; 0; 0 | 3; 2; 1; 1; 1; 1; 1 | Median; 4; 4; 4; 4 |
| 17C029 | AAVhu68.CB7.CI.hIDUAcoV1.rBG | 0; 0; 0; 0; 0; 0; 0; 0; 0; 0; 0; 0; 0; 0; 0; 0; 0 | 1; 1; 1; 1; 1; 1; 1 | Median; 0; 0; 0; 0 |
| 17C020 | AAVhu68.CB7.CI.hIDUAcoV1.rBG + steroids | 0; 0; 0; 0; 0; 0; 0; 0; 0; 1; 1; 0; 0; 0; 1; 1 | 3; 2; 3; 3; 3; 4; 4; 4; 4 | Median; 2; 3; 2; 1 |
| 17-167 | AAVhu68.CB7.CI.hIDUAcoV1.miR183.rBG | 0; 0; 0; 0; 0; 0; 0; 0; 0; 0; 0; 0; 0 | 0; 0; 0; 0; 0; 0; 0; 0; 0 | Median; 0; 0; 0; 0 |
| 17-215 | AAVhu68.CB7.CI.hIDUAcoV1.miR183.rBG | 0; 0; 0; 0; 0; 0; 0; 0; 0; 0; 0; 0; 0; 0 | 0; 0; 0; 0; 0; 0; 0; 0; 0 | Median; 0; 0; 0; 0 |
| 17-102 | AAVhu68.CB7.CI.hIDUAcoV1.miR183.rBG | 0; 0; 0; 0; 0; 0; 0; 0; 0; 0; 0; 0; 0; 0 | 0; 0; 0; 0; 0; 0; 0; 0; 0; 0; 0 | Median; 0; 0; 0; 0 |

TABLE 3

Blood chemistry in NHP injected ICM with AAV.hIDUA vectors

| Animal # and group | Timepoint | Total Protein | Albumin | Globulim | A/G Ratio | AST | ALT | Alk Prosphatase | GGT | Total Bilirubin | BUN | Creatinine |
|---|---|---|---|---|---|---|---|---|---|---|---|---|
| | | g/ dL | g/ dL | g/ dL | - | IU /L | IU/ L | IU/L | IU/ L | mg/ dL | mg/d L | mg/ dL |
| 17C024 AAVhu6 8.hIDUA | Baseline | 5.6 | 3.8 | 1.8 | 2.1 | 33 | 40 | 703 | 64 | 0.1 | 27 | 0.6 |
| | D0 | 6.0 | 3.7 | 2.3 | 1.6 | 28 | 36 | 682 | 61 | 0.1 | 23 | 0.4 |
| | D7 | 6.1 | 3.9 | 2.2 | 1.8 | 30 | 33 | 680 | 64 | 0.1 | 30 | 0.5 |
| | D21 | 5.9 | 3.7 | 2.2 | 1.7 | 25 | 22 | 825 | 66 | 0.1 | 21 | 0.5 |
| | D35 | 5.9 | 3.4 | 2.5 | 1.4 | 32 | 24 | 777 | 74 | 0.1 | 22 | 0.5 |
| | D60 | 5.9 | 3.7 | 2.2 | 1.7 | 28 | 30 | 815 | 74 | 0.1 | 28 | 0.4 |
| | D90 | 5.5 | 3.6 | 1.9 | 1.9 | 29 | 25 | 689 | 76 | 0.1 | 24 | 0.6 |
| 17C031 AAVhu6 8.hIDUA | Baseline | 5.7 | 3.8 | 1.9 | 2.0 | 35 | 39 | 539 | 36 | 0.1 | 27 | 0.5 |
| | D0 | 6.5 | 4.0 | 2.5 | 1.6 | 37 | 43 | 570 | 47 | 0.2 | 23 | 0.4 |
| | D7 | 6.3 | 3.9 | 2.4 | 1.6 | 27 | 39 | 547 | 45 | 0.1 | 26 | 0.5 |
| | D21 | 6.6 | 3.9 | 2.7 | 1.4 | 33 | 34 | 476 | 39 | 0.1 | 25 | 0.6 |
| | D35 | 6.3 | 3.4 | 2.9 | 1.2 | 32 | 31 | 591 | 42 | 0.1 | 24 | 0.5 |
| | D60 | 6.2 | 4.0 | 2.2 | 1.8 | 32 | 33 | 545 | 45 | 0.1 | 25 | 0.4 |
| | D90 | 6.0 | 3.6 | 2.4 | 1.5 | 34 | 27 | 478 | 43 | 0.1 | 24 | 0.5 |
| 17C029 AAVhu6 8.hIDUA | Baseline | 6.8 | 3.6 | 3.2 | 1.1 | 41 | 43 | 574 | 74 | 0.1 | 27 | 0.7 |
| | D0 | 6.9 | 4.0 | 2.9 | 1.4 | 27 | 40 | 607 | 79 | 0.1 | 18 | 0.7 |
| | D7 | 7.1 | 3.8 | 3.3 | 1.2 | 29 | 40 | 503 | 71 | 0.1 | 24 | 0.6 |
| | D21 | 6.9 | 3.9 | 3.0 | 1.3 | 49 | 38 | 557 | 77 | 0.1 | 15 | 0.6 |
| | D35 | 6.6 | 3.6 | 3.0 | 1.2 | 30 | 33 | 555 | 76 | 0.1 | 19 | 0.6 |
| | D60 | 6.3 | 4.0 | 2.3 | 1.7 | 38 | 43 | 545 | 86 | 0.1 | 22 | 0.6 |
| | D90 | 6.2 | 3.7 | 2.5 | 1.5 | 31 | 23 | 506 | 81 | 0.1 | 20 | 0.6 |
| 17C016 AAVhu6 8.hIDUA + | Baseline | 6.0 | 3.9 | 2.1 | 1.9 | 42 | 35 | 623 | 59 | 0.2 | 16 | 0.5 |
| | D0 | 6.8 | 4.1 | 2.7 | 1.5 | 33 | 58 | 588 | 59 | 0.2 | 16 | 0.4 |
| | D7 | 6.3 | 3.9 | 2.4 | 1.6 | 26 | 28 | 444 | 51 | 0.1 | 20 | 0.5 |
| | D21 | 6.4 | 3.7 | 2.7 | 1.4 | 29 | 23 | 393 | 44 | 0.2 | 16 | 0.6 |
| steroids | D35 | 6.9 | 3.9 | 3.0 | 1.3 | 32 | 29 | 364 | 41 | 0.1 | 21 | 0.5 |
| | D60 | 6.5 | 3.8 | 2.7 | 1.4 | 41 | 29 | 525 | 63 | 0.2 | 23 | 0.5 |
| | D90 | 5.7 | 3.6 | 2.1 | 1.7 | 32 | 29 | 752 | 79 | 0.1 | 12 | 0.5 |
| 17C019 AAVhu6 8.hIDUA + Steroids | Baseline | 5.5 | 3.4 | 2.1 | 1.6 | 35 | 42 | 338 | 49 | 0.1 | 31 | 0.6 |
| | D0 | 6.3 | 3.7 | 2.6 | 1.4 | 32 | 39 | 383 | 49 | 0.1 | 19 | 0.6 |
| | D7 | 6.1 | 3.6 | 2.5 | 1.4 | 26 | 34 | 322 | 47 | 0.1 | 30 | 0.7 |
| | D21 | 6.0 | 3.3 | 2.7 | 1.2 | 28 | 32 | 332 | 53 | 0.1 | 19 | 0.7 |
| | D35 | 5.9 | 3.5 | 2.4 | 1.5 | 42 | 38 | 378 | 49 | 0.1 | 27 | 0.6 |
| | D60 | 5.6 | 3.6 | 2.0 | 1.8 | 35 | 36 | 437 | 61 | 0.2 | 23 | 0.5 |
| | D90 | 5.6 | 3.6 | 2.0 | 1.8 | 41 | 34 | 650 | 73 | 0.2 | 21 | 0.6 |
| 17C020 AAVhu6 8.hIDUA + steroids | Baseline | 6.1 | 4.1 | 2.0 | 2.1 | 26 | 33 | 641 | 87 | 0.1 | 21 | 0.7 |
| | D0 | 6.5 | 4.1 | 2.4 | 1.7 | 21 | 25 | 538 | 61 | 0.1 | 12 | 0.6 |
| | D7 | 6.9 | 4.0 | 2.9 | 1.4 | 21 | 28 | 463 | 58 | 0.1 | 21 | 0.7 |
| | D21 | 6.6 | 4.0 | 2.6 | 1.5 | 23 | 29 | 456 | 55 | 0.1 | 13 | 0.5 |
| | D35 | 6.2 | 3.7 | 2.5 | 1.5 | 22 | 26 | 309 | 54 | 0.1 | 20 | 0.6 |
| | D60 | 6.2 | 4.1 | 2.1 | 2.0 | 26 | 27 | 516 | 90 | 0.1 | 19 | 0.5 |
| | D90 | 6.1 | 3.8 | 2.3 | 1.7 | 23 | 22 | 605 | 82 | 0.1 | 16 | 0.6 |
| 17-167 AAVhu6 8.hIDUA-miR-183 | Baseline | 6.6 | 3.9 | 2.7 | 1.4 | 24 | 21 | 764 | 112 | 0.1 | 18 | 0.5 |
| | D0 | 6.7 | 4.1 | 2.6 | 1.6 | 27 | 21 | 606 | 88 | 0.1 | 20 | 0.4 |
| | D7 | 6.6 | 3.9 | 2.7 | 1.4 | 31 | 27 | 606 | 87 | 0.1 | 24 | 0.6 |
| | D21 | 6.8 | 4.2 | 2.6 | 1.6 | 29 | 27 | 584 | 102 | 0.1 | 22 | 0.5 |
| | D35 | 6.6 | 4.0 | 2.6 | 1.5 | 35 | 34 | 642 | 93 | 0.1 | 16 | 0.5 |
| | D60 | 6.0 | 3.7 | 2.3 | 1.6 | 37 | 25 | 689 | 79 | 0.1 | 15 | 0.6 |
| | D90 | 6.6 | 3.9 | 2.7 | 1.4 | 24 | 21 | 764 | 112 | 0.1 | 18 | 0.5 |
| 17-215 | Baseline | 6.1 | 3.4 | 2.7 | 1.3 | 24 | 34 | 536 | 51 | 0.1 | 22 | 0.5 |
| | D0 | 6.5 | 3.6 | 2.9 | 1.2 | 29 | 50 | 568 | 54 | 0.2 | 21 | 0.5 |
| AAVhu6 8.hIDUA-miR-183 | D7 | 6.3 | 3.7 | 2.6 | 1.4 | 24 | 35 | 499 | 46 | 0.1 | 27 | 0.6 |
| | D21 | 6.2 | 3.5 | 2.7 | 1.3 | 31 | 37 | 526 | 58 | 0.1 | 21 | 0.5 |
| | D35 | 6.0 | 3.4 | 2.6 | 1.3 | 33 | 47 | 619 | 57 | 0.1 | 18 | 0.4 |
| | D60 | 6.1 | 3.7 | 2.4 | 1.5 | 40 | 50 | 628 | 45 | 0.2 | 18 | 0.5 |
| | D90 | 6.3 | 3.9 | 2.4 | 1.6 | 27 | 42 | 856 | 55 | 0.1 | 17 | 0.6 |
| 17-102 AAVhu6 8.hIDUA-miR-183 | Baseline | 6.7 | 3.8 | 2.9 | 1.3 | 24 | 31 | 613 | 57 | 0.1 | 20 | 6.7 |
| | D0 | 7.0 | 3.6 | 3.4 | 1.1 | 32 | 35 | 632 | 57 | 0.1 | 13 | 7.0 |
| | D7 | 6.9 | 3.9 | 3.0 | 1.3 | 31 | 25 | 601 | 57 | 0.1 | 16 | 6.9 |
| | D21 | 7.0 | 3.8 | 3.2 | 1.2 | 31 | 30 | 611 | 65 | 0.1 | 20 | 7.0 |
| | D35 | 6.8 | 3.8 | 3.0 | 1.3 | 28 | 29 | 684 | 59 | 0.1 | 20 | 6.8 |
| | D60 | 6.4 | 3.8 | 2.6 | 1.5 | 34 | 36 | 588 | 44 | 0.1 | 20 | 6.4 |
| | D90 | 7.0 | 4.0 | 3.0 | 1.3 | 27 | 28 | 576 | 41 | 0.1 | 18 | 7.0 |

TABLE 4

Complete blood count in NHP injected ICM with AAV.hIDUA vectors

| Animal # and group | Timepoint | WBC | RBC | HGB | HCT | Platelet | Neutrophils | Lymphocytes | Monocytes | Eosinophils | Basophils |
|---|---|---|---|---|---|---|---|---|---|---|---|
| | | x10³/ µL | x10⁶/ µL | g/dL | % | x10³/ µL | /µL | /µL | /µL | /µL | /µL |
| 17C024 AAVhu68. hIDUA | Baseline | 8.6 | 6.1 | 13.1 | 43 | 305 | 2838 | 5246 | 344 | 172 | 0 |
| | D0 | 6.1 | 5.5 | 12.7 | 40 | 304 | 3294 | 2562 | 183 | 61 | 0 |
| | D7 | 5.3 | 5.1 | 11.2 | 38 | 343 | 1060 | 3869 | 265 | 106 | 0 |
| | D21 | 6.7 | 5.7 | 12.8 | 42 | 289 | 1742 | 4422 | 335 | 201 | 0 |
| | D35 | 5.6 | 5.6 | 12.7 | 42 | 377 | 1792 | 3584 | 168 | 56 | 0 |
| | D60 | 6.6 | 5.8 | 13.0 | 42 | 356 | 1716 | 4488 | 198 | 198 | 0 |
| | D90 | 5.4 | 5.7 | 13.1 | 41 | 399 | 1674 | 3456 | 162 | 108 | 0 |
| 17C031 AAVhu68. hIDUA | Baseline | 11.9 | 5.3 | 12.6 | 41 | 386 | 4879 | 6188 | 476 | 357 | 0 |
| | D0 | 5.5 | 5.2 | 12.5 | 41 | 366 | 2255 | 2970 | 165 | 110 | 0 |
| | D7 | 14.4 | 4.8 | 11.2 | 39 | 357 | 8064 | 5040 | 864 | 432 | 0 |
| | D21 | 10.7 | 5.4 | 12.8 | 43 | 410 | 5136 | 4815 | 428 | 321 | 0 |
| | D35 | 9.7 | 5.3 | 12.8 | 43 | 267 | 4559 | 4559 | 388 | 194 | 0 |
| | D60 | 8.3 | 5.3 | 12.7 | 42 | 390 | 2988 | 4731 | 332 | 249 | 0 |
| | D90 | 8.4 | 4.9 | 12.1 | 39 | 414 | 4284 | 2940 | 672 | 420 | 84 |
| 17C029 AAVhu68. hIDUA | Baseline | 15.5 | 5.9 | 12.7 | 43 | 561 | 4495 | 9610 | 775 | 620 | 0 |
| | D0 | 15.1 | 5.7 | 12.6 | 41 | 493 | 9362 | 4681 | 755 | 302 | 0 |
| | D7 | 11.2 | 5.8 | 12.7 | 43 | 576 | 2800 | 7280 | 784 | 336 | 0 |
| | D21 | 10.6 | 5.8 | 12.6 | 42 | 496 | 4982 | 4770 | 530 | 318 | 0 |
| | D35 | 12.0 | 5.8 | 13.1 | 44 | 511 | 2520 | 8280 | 600 | 600 | 0 |
| | D60 | 11.6 | 5.8 | 13.6 | 43 | 497 | 3480 | 7192 | 696 | 232 | 0 |
| | D90 | 19.6 | 5.7 | 12.9 | 42 | 283 | 14896 | 3528 | 980 | 196 | 0 |
| 17C016 AAVhu68. hIDUA + steroids | Baseline | 10.2 | 5.9 | 13.5 | 44 | 235 | 1734 | 7752 | 306 | 408 | 0 |
| | D0 | 8.9 | 5.6 | 12.9 | 44 | 353 | 3382 | 5073 | 356 | 89 | 0 |
| | D7 | 9.6 | 5.5 | 12.3 | 41 | 346 | 2976 | 5952 | 576 | 96 | 0 |
| | D21 | 11.9 | 5.4 | 12.4 | 41 | 424 | 2856 | 8330 | 595 | 119 | 0 |
| | D35 | 7.9 | 5.4 | 12.7 | 43 | 352 | 4977 | 2528 | 316 | 79 | 0 |
| | D60 | 8.7 | 5.6 | 12.8 | 43 | 380 | 2610 | 5655 | 348 | 87 | 0 |
| | D90 | 8.4 | 5.4 | 12.7 | 41 | 357 | 2940 | 4956 | 420 | 84 | 0 |
| 17C019 | Baseline | 10.7 | 5.5 | 13.4 | 40 | 257 | 6099 | 4066 | 214 | 321 | 0 |
| | D0 | 10.6 | 6.4 | 13.9 | 49 | 358 | 7208 | 2650 | 318 | 424 | 0 |
| AAVhu68. hIDUA + Steroids | D7 | 10.8 | 5.9 | 13.1 | 44 | 286 | 3564 | 6048 | 108 0 | 108 | 0 |
| | D21 | 12.3 | 5.7 | 12.9 | 41 | 462 | 5658 | 5904 | 492 | 246 | 0 |
| | D35 | 12.4 | 5.9 | 13.4 | 45 | 297 | 7812 | 3720 | 620 | 248 | 0 |
| | D60 | 8.6 | 5.8 | 13.4 | 43 | 373 | 3612 | 4386 | 430 | 172 | 0 |
| | D90 | 11.1 | 5.5 | 13.0 | 41 | 349 | 8214 | 2442 | 333 | 111 | 0 |
| 17C020 AAVhu68. hIDUA + steroids | Baseline | 14.6 | 6.5 | 14.1 | 47 | 357 | 4818 | 8760 | 584 | 438 | 0 |
| | D0 | 15.4 | 6.1 | 13.7 | 44 | 339 | 8778 | 5390 | 616 | 616 | 0 |
| | D7 | 14.0 | 6.0 | 13.4 | 43 | 378 | 4060 | 8680 | 840 | 420 | 0 |
| | D21 | 14.4 | 5.7 | 12.9 | 42 | 327 | 6336 | 7056 | 720 | 144 | 144 |
| | D35 | 11.1 | 6.2 | 13.9 | 45 | 369 | 2664 | 7770 | 444 | 222 | 0 |
| | D60 | 10.8 | 6.2 | 14.0 | 45 | 380 | 4104 | 5940 | 324 | 432 | 0 |
| | D90 | 7.2 | 5.9 | 13.5 | 44 | 296 | 2376 | 4536 | 216 | 72 | 0 |
| 17-167 AAVhu68. hIDUA-miR-183 | Baseline | 10.6 | 5.4 | 13.7 | 44 | 245 | 1802 | 8162 | 530 | 106 | 0 |
| | D0 | 7.2 | 5.1 | 12.2 | 40 | 373 | 936 | 5904 | 288 | 72 | 0 |
| | D7 | 7.6 | 4.9 | 12.2 | 39 | 258 | 1140 | 6080 | 304 | 76 | 0 |
| | D21 | 10.2 | 5.5 | 13.4 | 45 | 270 | 3468 | 5712 | 714 | 102 | 204 |
| | D35 | 10.6 | 5.3 | 13.2 | 42 | 275 | 5406 | 4664 | 424 | 106 | 0 |
| | D60 | 9.5 | 5.2 | 13.4 | 42 | 115 | 3515 | 5510 | 380 | 95 | 0 |
| | D90 | 10.6 | 5.4 | 13.7 | 44 | 245 | 1802 | 8162 | 530 | 106 | 0 |
| 17-215 AAVhu68. hIDUA-miR-183 | Baseline | 12.9 | 5.8 | 12.7 | 44 | 380 | 2709 | 9546 | 387 | 258 | 0 |
| | D0 | 10.0 | 5.5 | 11.9 | 40 | 374 | 3600 | 5900 | 300 | 200 | 0 |
| | D7 | 11.2 | 5.4 | 12.0 | 40 | 423 | 2800 | 7616 | 560 | 224 | 0 |
| | D21 | 11.8 | 5.2 | 11.3 | 39 | 375 | 2242 | 9086 | 236 | 118 | 118 |
| | D35 | 12.5 | 5.6 | 12.4 | 42 | 337 | 5125 | 6625 | 500 | 250 | 0 |
| | D60 | 10.3 | 5.4 | 12.1 | 39 | 315 | 3811 | 5974 | 309 | 206 | 0 |
| | D90 | 13.7 | 5.3 | 12.1 | 38 | 415 | 8494 | 4795 | 274 | 137 | 0 |
| 17-102 AAVhu68. hIDUA-miR-183 | Baseline | 5.3 | 6.0 | 14.0 | 46 | 518 | 530 | 3816 | 371 | 530 | 53 |
| | D0 | 12.4 | 5.9 | 13.5 | 44 | 478 | 6944 | 4588 | 496 | 372 | 0 |
| | D7 | 9.2 | 5.8 | 13.1 | 44 | 501 | 3312 | 4968 | 368 | 552 | 0 |
| | D21 | 9.0 | 5.9 | 14.0 | 45 | 510 | 2250 | 5940 | 540 | 270 | 0 |
| | D35 | 9.4 | 6.4 | 14.5 | 49 | 409 | 3760 | 4888 | 564 | 188 | 0 |
| | D60 | 9.1 | 6.2 | 14.6 | 46 | 410 | 3276 | 5005 | 455 | 364 | 0 |
| | D90 | 7.8 | 5.9 | 14.4 | 44 | 528 | 3042 | 4212 | 312 | 234 | 0 |

TABLE 5

CSF white blood cell counts (cells per µL) in NHP injected ICM with AAV.hIDUA vectors

| Group | Animal # | Day 0 | Day 21 | Day 35 | Day 60 | Day 90 |
|---|---|---|---|---|---|---|
| AAVhu68. hIDUA | 17C024 | 0 | 1 | 2 | 1 | 2 |
| | 17C031 | 0 | 1 | 2 | 1 | 1 |
| | 17C029 | 0 | 0 | 0 | 0 | 0 |
| AAVhu68. hIDUA + steroids | 17C016 | 0 | 0 | 0 | 0 | 3 |
| | 17C019 | 0 | 1 | 2 | 1 | 0 |
| | 17C020 | 0 | 0 | 5 | 1 | Blood contamination |
| AAVhu68. hIDUA-miR-183 | 17-167 | 0 | 2 | 3 | 1 | 1 |
| | 17-215 | 0 | 1 | 2 | 1 | 1 |
| | 17-102 | 0 | 3 | 7 | 2 | 0 |

### AAV-Induced DRG Toxicity in NHPs Occurs Via Neuronal Apoptosis

In order to investigate the mechanism of neuronal degeneration in DRG, we performed immunohistochemistry (IHC) for markers of cellular apoptosis and unfolded protein response (UPR). Initial studies focused on the activation of capspase-3 which is a downstream marker of apoptosis. DRG of animals that exhibited neuronal degeneration based on H&E evaluation showed positive IHC staining for activated caspase-3 along with increases in cellular infiltrates. DRG from a non-AAV 6 injected animal and spleen served as negative and positive controls, respectively. Caspase-3 positive neurons in NRGs were higher in the GFP groups as compared to the hIDUA groups. In each case, inclusion of the miR-183 target sequence reduced the number of cells with activated caspase-3. We evaluated apoptosis induced by adaptive or innate immunity, in what is referred to as the extrinsic pathway, by evaluating up-regulation of caspase-8 by IHC. Degenerating neuronal cell bodies across all vector groups were negative for activated caspase-8 while infiltrating cells were strongly positive for caspase-8, which served as an internal positive control. Sections were also evaluated for activation of caspase-9 which is a common marker of intrinsic apoptosis. IHC demonstrated caspace-9 in multiple degenerating neuronal cell bodies of DRG in an animal that received AAVhu68.eGFP); however, none were observed in animals that received AAVhu68.eGFP.miRNA and exhibited neuronal degeneration. There was no clear increase in caspase-9 in neurons from animals that received AAVhu68.hIDUA vectors with or without miR-183, however this may simply be a function of decreased incidence of lesions observed with these vectors compared to AAVhu68.eGFP which reduces the likeliness of finding neurons at the right stage of degeneration on histological sections. Intrinsic pathway of apoptosis, which is considered the major mechanism of apoptosis, is mediated via the release of cytochrome C due to increased membrane permeability of the mitochondria and activation of caspase 9. Apoptosis via the unfolded protein response (UPR) occurs through the intrinsic pathway.

In order to support the proposed mechanism of toxicity due to protein overexpression from high levels of transgene product, IHC for activating transcription factor 6 (ATF6) was performed. The UPR triggers ATF6 activation in the Golgi to generate cytosolic fragments which migrate to the nucleus to activate transcription of ER-associated binding elements. Interestingly, IHC for ATF6 was multifocally positive in the cytoplasm of neuronal and perineuronal satellite cells in the DRG of animals that received AAVhu68.eGFP, AAVhu.68.hIDUA, and AAVhu68.eGFP.miR-183 which corresponded to lesion severity. By contrast, animals that received AAVhu68.hIDUA.miR-183, as well as the naive non-AAV-injected control NHP, were diffusely negative for ATF6. The highest level of ATF6 expression was observed in the animal injected with AAVhu68.eGFP followed by AAVhu68.hIDUA and AAVhu68.eGFP.miR-183. Consistent with the overall study findings, animals that received vector with the miR-183 showed decreased ATF6 positivity indicating decreased cellular stress.

Toxicity of DRGs is likely to occur with any therapy that relies on high systemic doses of vector or direct delivery of vector into the CSF. This safety concern is limited to primates and has usually been asymptomatic. However, DRG toxicity can cause substantial morbidity such as ataxia due to proprioceptive defects (Hinderer, C., et al. Hum Gene Ther. 29(3):285-298, 2018) or intractable neuropathic pain. The U.S. Food & Drug Administration recently paused an intrathecal AAV9 clinical trial for late-onset SMA due to NHP DRG toxicity, which underscores how this risk may limit the development of AAV therapies (Novartis. Novartis announces AVXS-101 intrathecal study update, 2019).

It was originally hypothesized that this toxicity was caused by destructive T-cell immunity to transduced neurons in DRGs directed towards foreign capsid or transgene epitopes. However, strong immune suppression regimens such as MMF and rapamycin did not prevent the toxicity in toxicology studies (Hordeaux, J., et al. Mol Ther Methods Clin Dev 10:68-78, 2018; Hordeaux, J., et al. Mol Ther Methods Clin Dev 10:79-88, 2018), nor did steroids in this study. The time course of delayed but not progressive DRG degeneration did not support the notion that adaptive immunity played a role. If cytotoxic T cells were involved, DRG degeneration and mononuclear cell infiltrates that began early and progressed over time would have been observed.

It may be that high levels of DRG transduction create cellular stress, which leads to degeneration in the highly transduced DRG neurons. Since toxicity can be prevented by suppressing transgene mRNA and protein expression, capsid or vector DNA cannot be the cellular stressors. Histological analysis demonstrated that degeneration was limited to DRG neurons that expressed the highest level of transgene protein. Neuron degeneration was also associated with caspase-3 and -9 activation, which suggest apoptosis caused by an intracellular source of stress as opposed to T-cell mediated cell death. Reduction of DRG degeneration by cell specific ablation of transgene expression via miR-183 suggests that it is overexpression of transgene derived mRNA or protein rather than capsid or vector DNA that this driving this process. Increase in ATF6 staining in neuron and satellite cells in animals receiving vectors without the miR targets compared to controls with miR targets or naive animals implicates the UPR, although the inciting mechanism may differ between non-secreted (GFP) versus secreted (IDUA) transgenes.

The time course of delayed but self-limited DRG neuronal degeneration is consistent with the notion that non-immune toxicity is restricted to a subset of highly transduced cells. It is unclear whether the DRG toxicity and axonopathy are reversible. After following adult animals for six months, consistent reductions in pathology have not been observed. The only experiment where DRG toxicity was observed in NHPs following ICM injection was when the vector was administered to one-month old macaques that were necropsied four years later (Hordeaux et al., 2019). It is possible that infant primates are resistant to DRG toxicity, or their DRG neurons have regenerative capacity, or the lesions regressed over this extended time period.

The findings presented support that DRG toxicity is caused by transgene overexpression. Therefore, the severity of DRG toxicity should be influenced by dose, promoter strength, and the nature of the transgene. It is still not understood why sensory neurons are one of the most efficiently transduced cells in primates. DRGs are easily accessed by systemically administered vectors because they reside outside of the CNS and have porous, fenestrated capillaries. Systemic vector could also access DRG neurons via retrograde transport after uptake from peripheral axons. The anatomy of sensory neuronal compartments that reside within the intrathecal space may promote high transduction of vectors delivered into the CSF. Axons of DRG neurons in the dorsal roots are exposed to CSF providing easy access to vector following ICM/LP administration. Open access of the subarachnoid space to the extracellular fluid of the DRG should allow direct contact of ICM/LP vector to the neuronal cell bodies and other cells of the DRG. Suppression of transgene expression within DRG neurons with miR-183 facilitated an analysis of transgene expression in other dells of the DRGs which should not be influenced by this miR. ISH reveled transgene mRNA in surrounding glial satellite cells that could suggest direct transduction (FIG. 6C). The functional consequence of transgene mRNA in glial cells is unknown.

Selectively inhibiting vector transgene expression should reduce and potentially eliminate DRG toxicity. The key for achieving this is a strategy for specifically extinguishing expression in DRG neurons without affecting expression elsewhere. There are currently no ways to achieve this specificity through capsid modifications or tissue-specific promoters. Including targets for miR-183 into the vector achieved the desired result of reducing/eliminating DRG toxicity without affecting vector manufacturing, potency, or biodistribution. Included in the hIDUA NHP study above was a group that received non-miR-183 vector with concomitant steroids - a standard approach for mitigating immune-mediated toxicity in AAV trials. DRG toxicity was not reduced in the steroid-treated group; in fact, there was a trend toward worsening toxicity. This experiment demonstrates the limitations of prophylactic steroids in AAV gene-therapy trials.

The modularity of this approach for diminishing DRG toxicity suggests its use in any AAV vector considered for CNS gene therapy where mitigating AAV-induced DRG toxicity is desirable. This approach can be used across a broad array of AAV vectors for therapeutic applications.

### Example 4: In Vitro and In Vivo Assessment of Expression Constructs With miR-183 Cluster Target Sequences

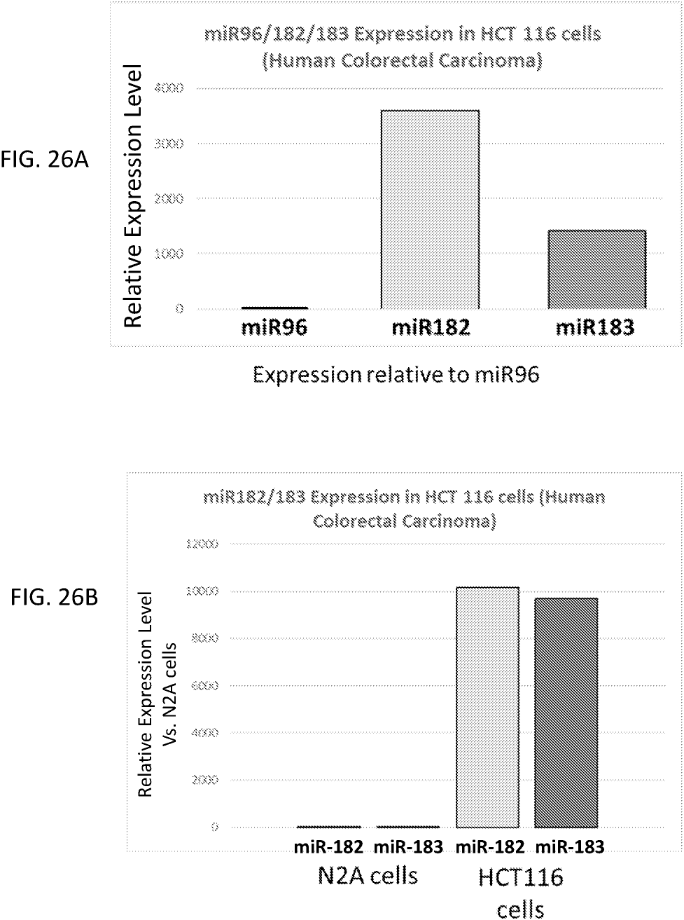

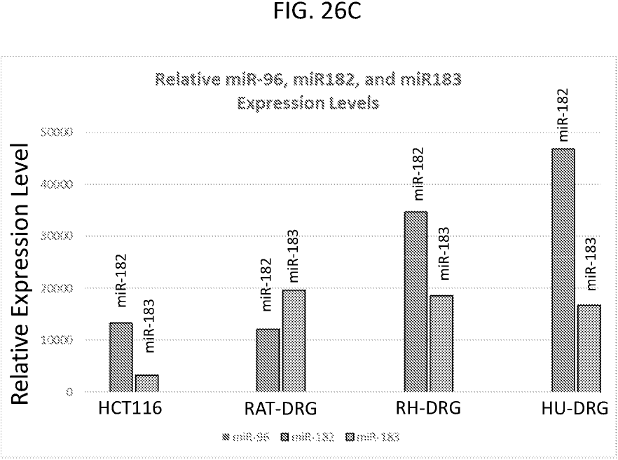

In vitro assays were used to evaluate the activity and specificity of constructs harboring miRNA target sequences. As described in Example 2 above, HEK293 cells (or another suitable cell line) can be co-transfected with a cis plasmid having the GFP transgene and plasmids expressing one or more miRNA, such as miR-182 and miR-183. The cis plasmids are designed with varying number of corresponding target miRNA sequences in the 3′UTR of the expression cassettes and alternative spacer sequences are introduced. At 72 hours post transfection, expression of GFP is quantified to determine relative levels of expression. Rat, rhesus, or human DRG cells can also be transduced to evaluate efficacy of various constructs. In addition, a screening assay was developed using the HCT 116 cell line. which expresses the miR-96, miR-182, and miR-183 (FIG. 26A and FIG. 26B).

Based on results of the in vitro studies, the suitable combination of sequences (including number of repeats) and spacers that reduce or eliminate expression of GFP are identified. For example, FIG. 24 shows the effect of including miR-183, mir-182, miR-96, or miR-96 on expression of GFP in the brain cortex following transduction with AAV-PHP.B.GFP vectors. An exemplary in vivo mouse study to evaluate CNS expression levels, including, for example, detargeting of DRG (i.e. reduction of GFP expression), is also provided in Example 2.

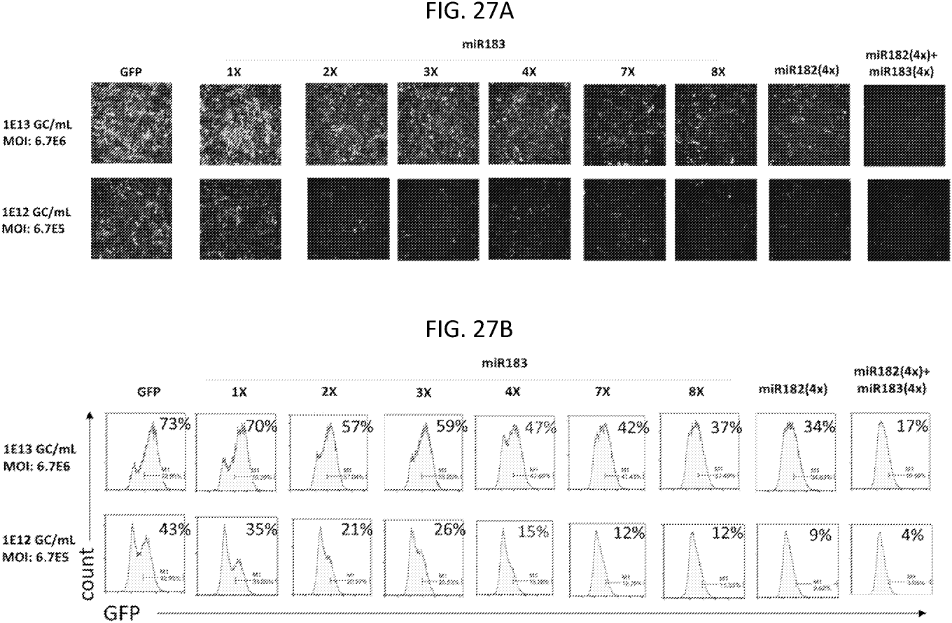

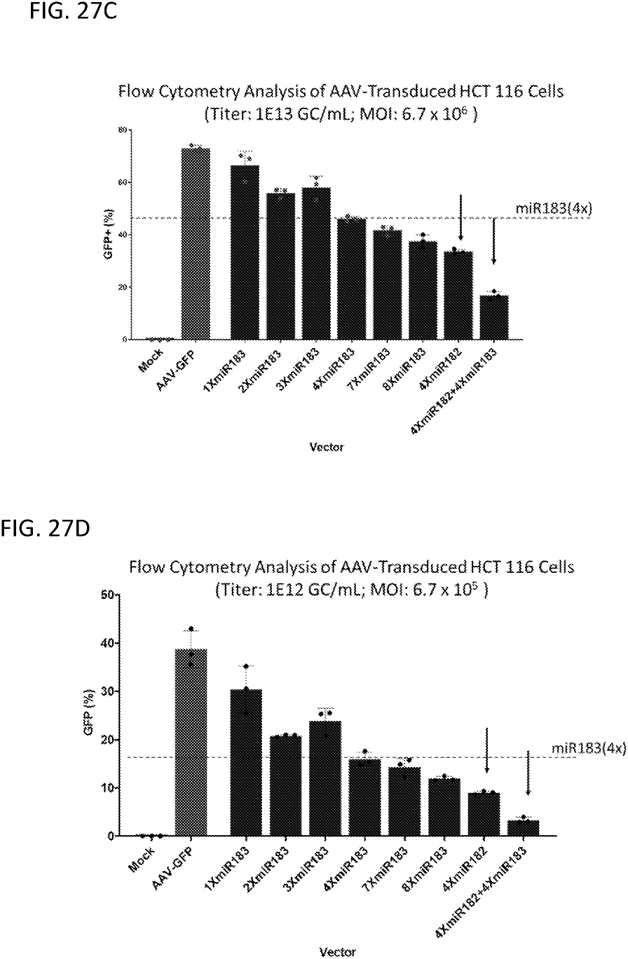

The HCT cell line is a suitable model reproducing a similar ratio of miR-183 / miR-182 compared to NHP and human DRG (FIG. 26C). FIG. 27A - FIG. 27D show results from transducing HCT116 cells with a vector having four miR-182 target sequences, as well as constructs having a combination of miR-182 and miR-183 target sequences (four miR-182 target sequences and four miR-183 target sequences). Decreased GFP expression was observed with increasing miR-183 target copies. Further, vectors having either four miR-182 target sequences or a combination of four miR-182 target sequences and four miR-182 target sequences lead to higher silencing (reduced GFP expression) versus a vector having four miR-183 target sequence (FIG. 27C and FIG. 27D).

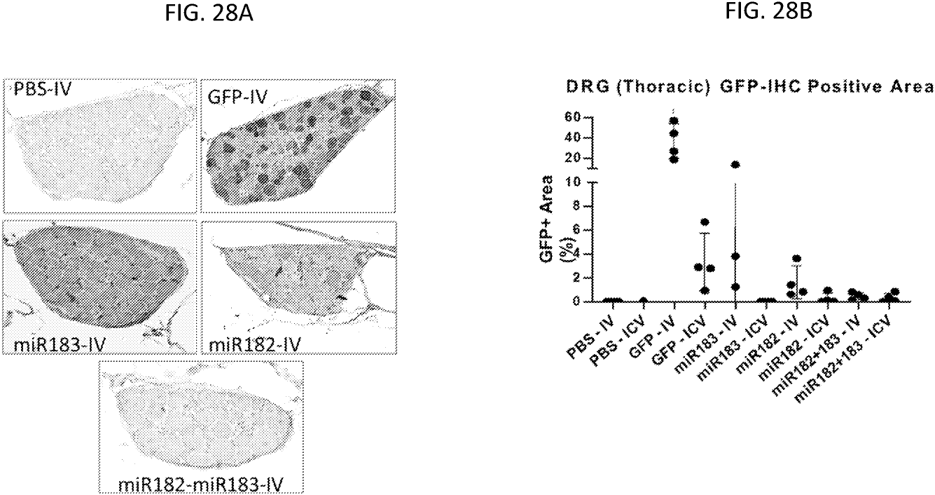

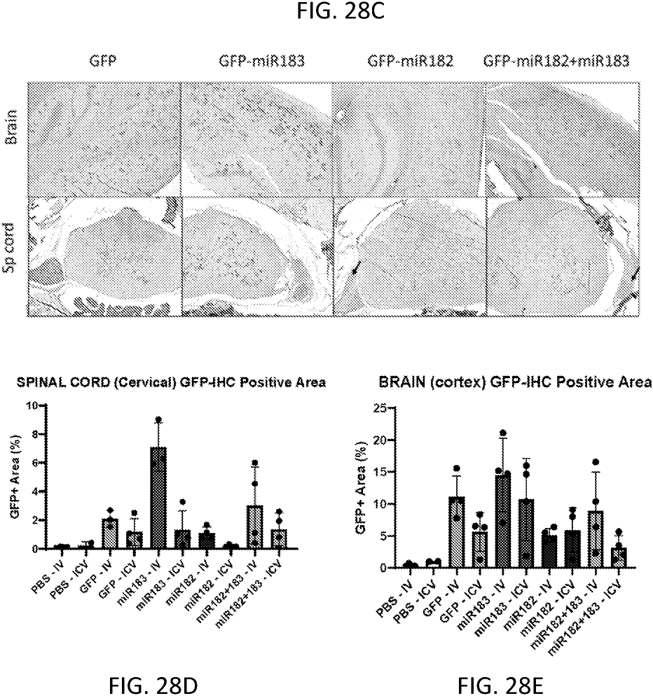

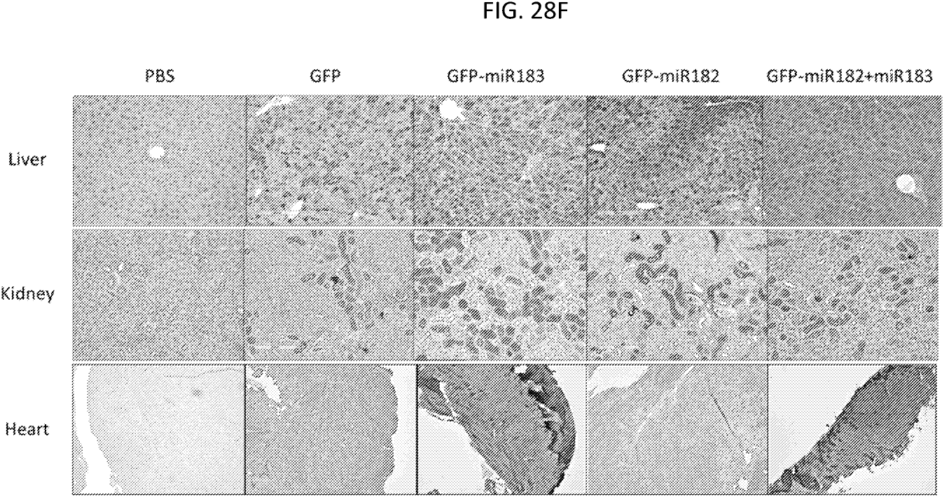

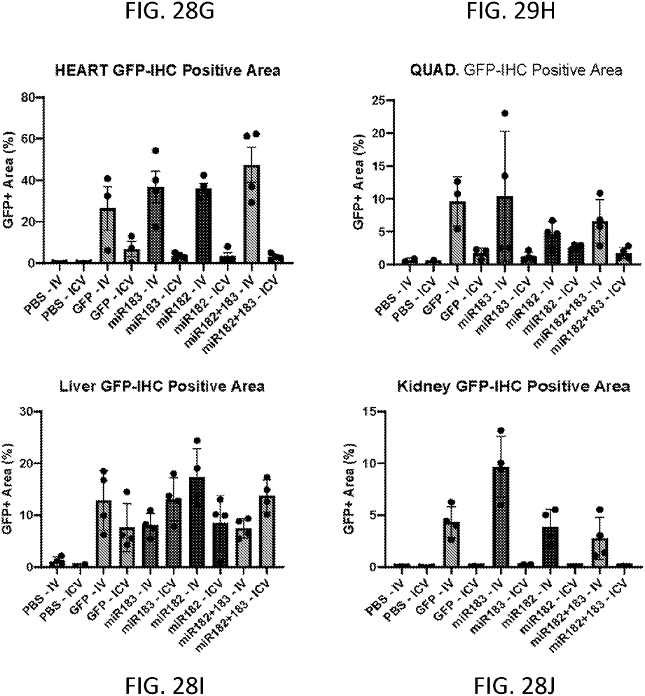

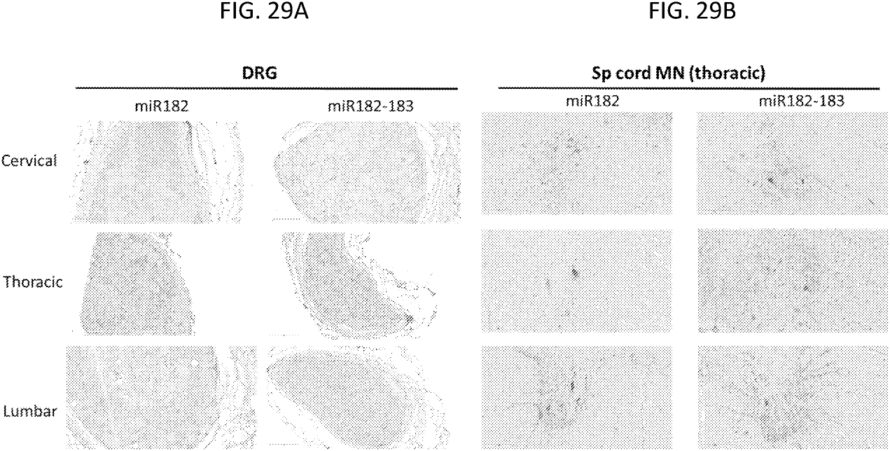

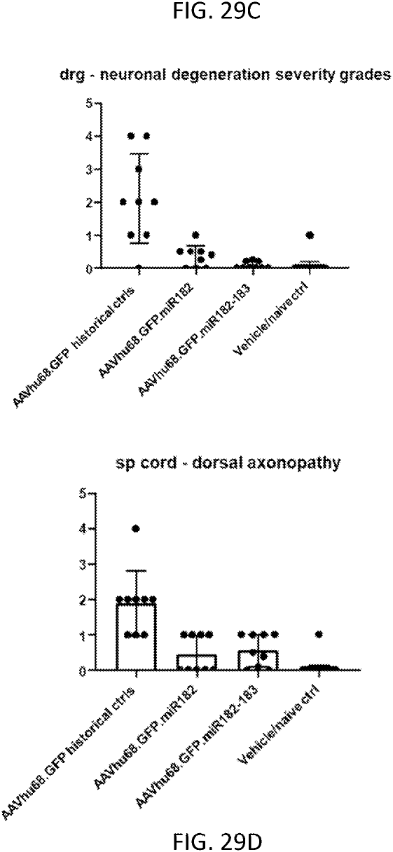

Constructs having miR-182 target sequences only and combinations of miR-182 and miR-183 target sequences that showed favorable reduced levels of expression in vitro were also evaluated in vivo. Following administration of AAVhu68 vectors, toxicity and levels of transgene expression (extent of detargeting) in cells of the CNS and DRG and was evaluated. Mice received 4×10¹² GC IV or 1×10¹¹ GC ICV of vectors that included an expression cassette with a GFP transgene and either no miR target sequences, four copies of a miR-183 target sequence, four copies of a miR-182 target sequence, or 4 copies of a miR-182 target sequence and four copies of a miR-183 target sequence (see also SEQ ID NO: 28). The results shown in FIG. 28A and FIG. 28B demonstrate that the constructs with miR-182 target sequences or miR-182 target sequences plus miR-183 target sequences silenced transgene expression in the DRG. However, transgene expression in the brain and spinal cord (FIG. 28C - FIG. 28E) and peripheral tissues (FIG. 28F - FIG. 28J) was preserved. NHP studies with the AAVhu68 vectors (ICM delivery of 3×10¹³) also demonstrated that the constructs with four copies of a miR-182 target sequence or a construct with four copies of a miR-182 target sequence and four copies of a miR-183 target sequence silenced transgene expression in DRG (FIG. 29A and FIG. 28B). Further, both constructs were associated with reduced DRG toxicity and dorsal axonopathy (FIG. 29C and FIG. 29D).

### Example 5: Detargeting of a Human Iduronate-2-Sulfatase (hIDS) Transgene for Treatment of Mucopolysaccharidosis Type II (MPS II)

One strategy for the treatment of MPS II (Hunter syndrome) is to functionally replace a patient’s defective iduronate-2-sulfatase via rAAV-based CNS-directed gene therapy (see, e.g., International Patent Application No. PCT/US2017/027770, which is incorporated by reference herein). To reduce DRG toxicity, AAV vector genomes for treatment of MPSII are modified by introducing miR target sequences. Accordingly, AAV vector genomes containing a hIDS coding sequence are designed with one, two, three, or four miR-183 target sequences. The effectiveness of DRG detargeting in vivo is measured, for example, following intrathecal administration of the AAV vector encoding hIDS to NHPs.

### Example 6: Detargeting of a SMN1 Transgene for Treatment of Spinal Muscular Atrophy (SMA)

SMA is an autosomal recessive disorder caused by mutations or deletion of the bSMN1 gene. Delivery of functional SMN protein via rAAV vectors has been effective for treatment of SMA but DRG toxicity has been observed. Suitable vectors include those described in International Patent Application No. PCT/US2018/019996, which is incorporated by reference herein, and Zolgensma®, an AAV9-based gene therapy). Reduction or elimination of DRG toxicity following delivery of AAV vectors encoding human SMN1 is achieved by incorporating miRNA target sequences, such as those recognized by miR-182 and miR-183, into the vector genome. Accordingly, AAV vectors, including those with AAV9 or AAVhu68 capsids, are generated having a nucleic acid sequence encoding a bSMN1 transcript in combination with one, two, three, or four miRNA target sequences. The target sequences are selected, for example, from miR-182 and miR-183 target sequences, or a combination thereof. DRG toxicity following IV or intrathecal administration of a hSMN1-expressing AAV vectors is evaluated in a NHP model.

### Example 7: Liver-Directed Gene Therapy Vectors Having miRNA Target Sequences

Where improved expression of a transgene in liver tissue is desirable for gene therapy, AAV vector genomes can be modified to include miRNA target sequences. For example, a rAAV designed to express a functional low-density lipoprotein receptor (hLDLR) gene and bearing an AAV8 capsid is suitable for treatment of treatment of familial hypercholesterolemia (FH) (see, e.g., International Patent Application No. PCT/US2016/065984, which is incorporated herein by reference). Enhanced expression of the hLDLR transgene in liver tissue is achieved using an rAAV with a vector genome having a hLDLR coding sequence in combination with one, two, three, or four miR-182 target sequences. Likewise, gene therapies for treatment of hemophilia A (Factor VIII) and hemophilia B (Factor IX) include vectors with tropism for the liver (see, e.g., International Patent Application No. PCT/US2017/027396 and International Patent Application No. PCT/US2017/027400, which are incorporated herein by reference). More effective delivery and expression of human factor VIII and factor IX in liver is achieved by delivering rAAVs with vectors genomes having one, two, three, or four miR-182 target sequences in combination with the transgene.

### Example 8: Assessment of DRG-Detargeting In Vector Genomes With Alternative Copies of miRNA Target Sequences

AAV9 vectors were designed encoding green fluorescent protein (eGFP) under the CB7 promoter as described previously. The expression cassettes were designed to contain a single miR-183 detargeting sequence, two copies of a miR-183 detargeting sequence, three copies of an miR-183 detargeting sequence, or eight copies of an miR-183 detargeting sequence in the 3′ UTR of the eGFP. These vectors are produced and titrated as described [Lock et al., Hum Gene Ther., October 2010; 21(10): 1259-1271]. Briefly, HEK293 cells are triple-transfected, the cells are lysed, and the vectors are harvested, concentrated, and purified as previously described. The purified vectors are titrated with droplet digital PCR using primers targeting the rabbit Beta-globin polyA sequence as previously described (Lock et al., Hum Gene Ther Methods; April 2014; 25(2): 115-125).

The sequences of a vector genome containing an eGFP transgene and one copy of the miR-183 are provided in SEQ ID NO: 20. An illustrative vector genome containing an eGFP transgene and two copies of a miR-183 detargeting sequence is provided in SEQ IDNO: 21. The sequences of a vector genome containing an eGFP transgene and 3 copies of the miR-183 are provided in SEQ ID NO: 22. An illustrative vector genome containing an eGFP transgene and four copies of a miR-183 detargeting sequence is provided in SEQ ID NO: 23. An illustrative vector genome containing an eGFP transgene and seven copies of a miR-183 detargeting sequence is provided in SEQ ID NO: 26. An illustrative vector genome containing an eGFP transgene and eight copies of a miR-183 detargeting sequence is provided in SEQ ID NO: 27. Alternatively, a vector genome has a combination of miR target sequences. For example, SEQ ID NO: 28 provides a vector genome that includes four copies of a miR-182 target sequence and four copies of a miR-183 target sequence.

AAV9 vectors, AAV9.CB7.CI.eGFP.rBG, AAV9.CB7.CI.eGFP.miR-183.rBG, and AAV9.CB7.CI.eGFP.miR-183.rBG were constructed as described in Example 1. The AAV9.CB7.CI.eGFP.miR-183.rBG and AAV9.CB7.CI.eGFP.miR-183.rBG vectors genomes include four copies of a miR-183 or miR-145 detargeting sequence in the 3′ UTR of the eGFP coding sequence and the effect on expression levels in drg and other tissue and cell types was assessed using the methods described in the preceding examples. FIG. 24A shows that the modified to include miR-145 targets showed decreased expression in heart compared to the control no-miR vector. The vector with 4X miR-183 targets showed increased GFP transduction in heart compared to the no-miR and miR-145 target vectors. FIG. 24B shows that the vector with 4X miR-183 targets showed increased GFP transduction in brain cortex and brainstem compared to the no-miR and miR-145 vectors.

### Example 9: rAAV Comprising miR-Detargeting Sequences Operably Linked to a Transgene Do Not Increase Expression of mir-183 Cluster-Regulated Genes

Human CACNA2D1 and CACNA2D2 genes (members encode voltage-gated calcium channel) are predicted targets of the miR-183 cluster (miR-183/-96/-182) and prior publications suggest a significant inverse correlation between all three miRNAs and CACNA2D1 and CACNA2D2 expression in DRG from human donors. See, e.g., Peng at al, “mirR-183 cluster scales mechanical pain sensitivity by regulating basal and neuropathic pain genes”. Science. 2017 Jun 16;356(6343): 1168-1171. doi: 10.1126/science.aam7671. Epub 2017 Jun 1. It has been reported that miR-183 downregulates CACNA2D expression. Thus, increased expression of CACNA2D would possibly result from a “sponge effect”, which would be expected to result in an increased sensitivity to pain and pressure.

Stock rAAVs containing a vector genome comprising eGFP or IDUA with or without 4xmiR-183 target sequences were diluted to 2.5 × 10¹²/mL with rat-DRG medium, and 0.25 ml of vector was added to each DRG-containing well of a 24-well-plate, after removing the old media. After 24 hours, media were removed and replaced with fresh media. The transductions were done in triplicates (i.e. 3 wells for AAV-GFP and 3 wells for AAV-GFP-miR-183 (2 wells for mock control). To enhance transductions, adenovirus AD5 (SignaGen Laboratories; Rockville, MD) was also added at an MOI of 10, along with the AAV vectors. RNA was isolated separately for each well and used for q-RT-PCRs (one reaction /well; in duplicates). Total RNA was extracted from the DRG cultures 72 hours following transductions.

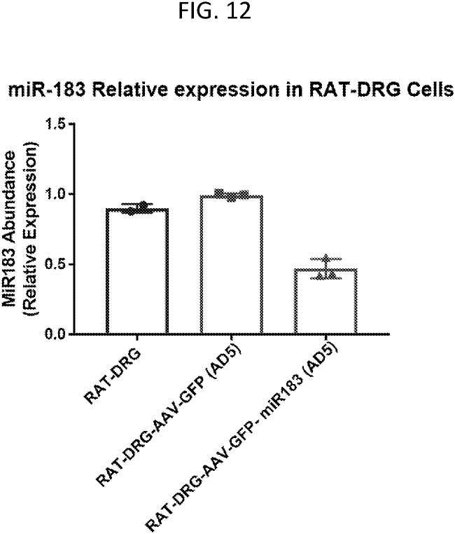

The expression levels of miR-183 and the potential sponge effect on target genes, CACNA2D1 and CACNA2D2, were determined using primers specific to Rat CACNA2D1 (Assay ID Rn01442580) and CACNA2D2 (Assay ID: Rn00457825). FIG. 11 shows results from transduction with various AAV9 vectors having an eGFP transgene with or without four copies of the miR-183 detargeting sequences at low (5 ×10⁵) or high (2.5 × 10⁸) concentration. The low and high doses without miR-183 were tested with or without adenovirus type 5 (Ad5) helper co-transfection at a multiplicity of infection (MOI) of 100 (for low dose AAV9-eGFP) or 10 (high dose AAV9-eGFP). All DRG neurons were transduced, and no visible sign of toxicity were observed. No GFP expression was observed in DRG neurons, while some expression was observed in fibroblast-like cells. The findings confirm repression of GFP transcription with the vector genomes having (x4)miR-183 target sequences.

### miR-183 Sponge-Effect Study in NHP

DRG (lumbar) and brain (frontal cortex) tissues were obtained from a non-human primate (NHP) rhesus monkeys study (19-04) in which animals had been administered AAV-IDUA or AAV-IDUA-4Xmir-183 vectors (n=3/group). A miRNeasy Mini Kit was used for total RNA isolation (Qiagen, Germantown, MD) and the extracted RNA were then reverse transcribed with TaqMan™ MicroRNA Reverse Transcription Kit (Applied Biosystems), according to the instruction of the protocol. Quantitative real-time polymerase chain reaction (qPCR) was performed to determine the abundance of miR-183 in different tissues, using the TaqMan MicroRNA Assay kit with primers specific to hsa-miR-183-5p (Assay ID 002269) and RNU6B (Assay ID 00193) (Applied Biosystems Inc., Foster City, CA, USA) following the manufacturer’s instructions. Similarly, the abundance of two of the direct targets of miR-183, namely CACNA2D1 and CACNA2D2 were measured using the TaqMan Gene Expression Assay kit with primers specific to CACNA2D1 (Assay ID Hs00984840) and CACNA2D2 (Assay ID: Hs01021049), respectively. Each qPCR assay was conducted in triplicate using cDNA derived from 100 ng total RNA from a biological replicate and analyzed by the comparative threshold cycle (Ct) method. The average expression level of miR-183 was normalized with RNU6B as an endogenous control gene, and the average level of CACNA2D1 and CACNA2D2 were normalized with GAPDH, using the 2^{-ΔΔCt} method (Schmittgen TD, Livak KJ. Analyzing real-time PCR data by the comparative C(T) method. Nat Protoc. 2008;3(6): 1101-8.).

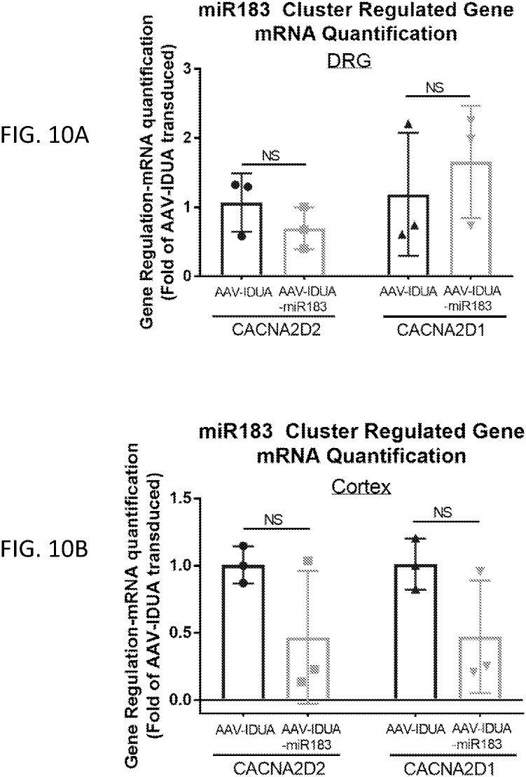

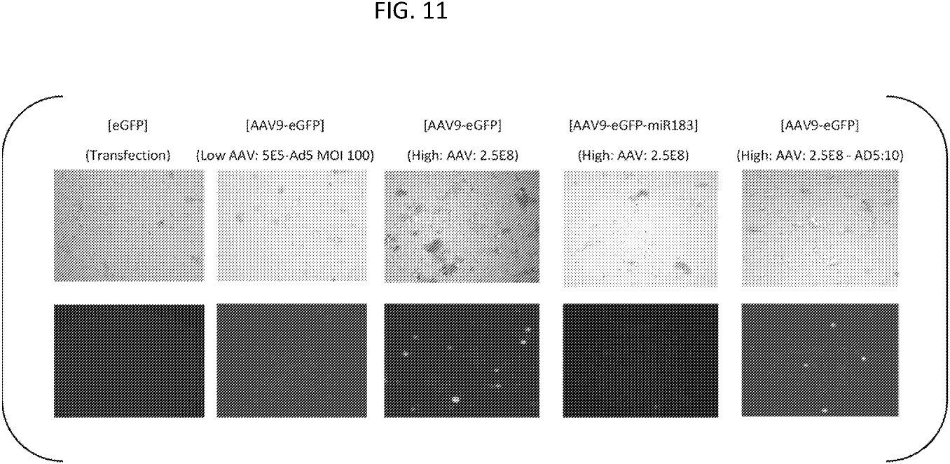

There was no increased expression of miR-183 cluster-regulated genes (CACNA2D1 or CACNA2D2), comparing AAV-IDUA or AAV-IDUA-miR-183 administered-animals, in either DRG (high miR-183 abundance) or frontal cortex (low miR-183 abundance) (FIG. 10A and FIG. 10B)

### Rat Neonatal Dorsal Root Ganglion (DRG) Neuron Cell Culture

Rat DRG Neurons (LONZA WALKERSVILLE INC) were thawed and added to 7 mL of recommended media (PNGM BulletKit: Primary Neuron Basal Medium containing 2 mM L-glutamine, 50 µg/ml Gentamicin/37 ng/ml Amphotericin, and 2% NSF-1). The 8 ml media containing ∼5.0E5 DRG neurons was then divided between 8 wells of a 24-well tissue culture plate that was coated with poly-D-lysine (30 µg/ml; Sigma) immediately before adding the cells. Cells were incubated for 4 hours in a 37° C., 5% CO₂ incubator and then the media was removed and replaced with fresh, pre-warmed medium. To inhibit Schwann cell proliferation, mitotic cell inhibitors (5 µl of 17.5 ug/ml uridine and 5 ul of 7.5 µg/mL 5-fluoro-2-deoxyuridine/ml of medium) were added after the initial 4 hours incubation. Cells were incubated at 37° C., 5% CO₂ with complete media change on day 5 and 50% media change every 3 days after that. After six days of initial culture, RAD DRG neurons were transduced with AAV vectors as described above.

FIG. 12 shows the effect of the miR-183 sponge effect study in rat DRG cells. The data show that miR-183 levels in rat DRG cells were decreased when cells were transduced with a AAV9-eGFP-mir-183 vector. The finding indicated engagement of the target sequences of the expressed GFP-miR-183 mRNA.

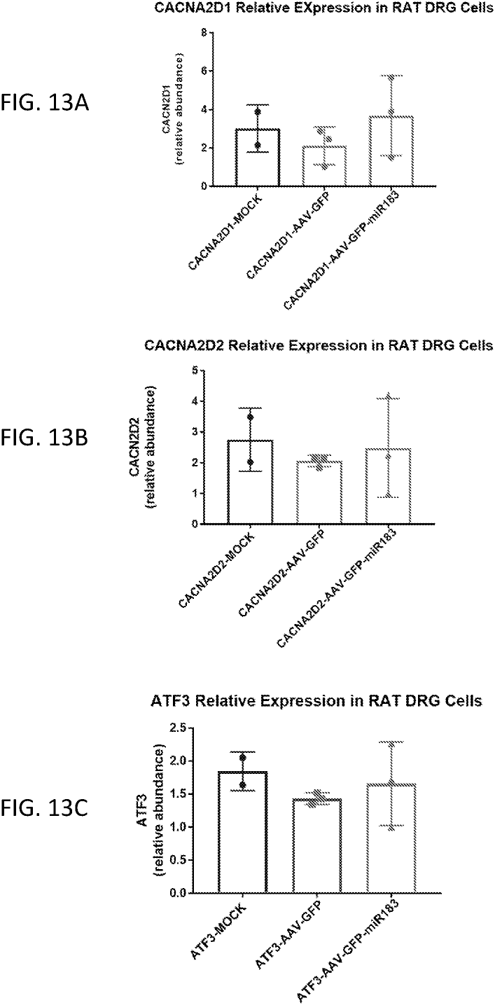

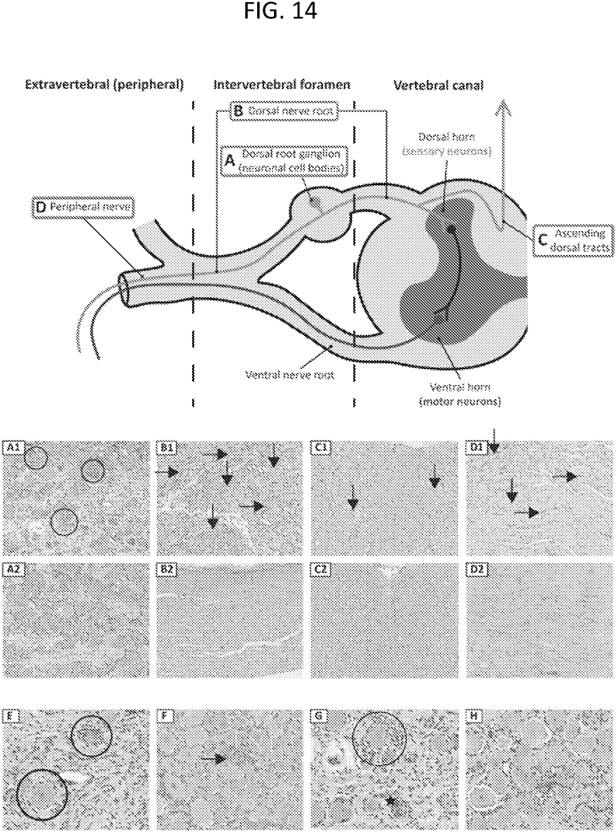

FIG. 13A and FIG. 13B show the effect of the miR-183 sponge effect study in rat DRG cells for three known miR-183 regulated transcripts. FIG. 13A shows relative expression CACANA2D1 in rat DRG cells following transduction with a mock vector, AAV-GFP, or AAV-GFP-miR-183 vector. FIG. 13B shows relative expression of CACANA2D2 in rat DRG cells following transduction with a mock vector, AAV-GFP, or AAV-GFP-miR-183 vector. FIG. 13C shows relative expression of ATF3 in rat DRG cells following transduction with a mock vector, AAV-GFP, or AAV-GFP-miR-183 vector. No change in relative expression of mRNA levels of these known miR-183 regulated transcripts. No difference was observed compared to mock wells untransduced and GFP-miR-183 transduced wells. These data demonstrate the absence of sponge effect in those cells. It is possible that either remaining levels of miR-183 are sufficient or other members of the cluster (miR-96 and/or miR-182) can compensate for the decreased availability of miR-183.

### Example 10: Meta-Analysis of DRG Pathology

The administration of adeno-associated virus (AAV) vectors to non-human primates (NHP) via the blood or cerebral spinal fluid (CSF) can lead to dorsal root ganglion (DRG) pathology. The pathology is minimal to moderate in most cases, clinically silent in affected animals, and characterized upon histopathological analysis by mononuclear cell infiltrate, neuronal degeneration, and secondary axonopathy of central and peripheral axons. We aggregated data from 33 nonclinical studies in 256 NHP and performed a meta-analysis of the severity of DRG pathology between different routes of administration, dose, time course, study conduct, age of the animals, sex, capsid, promoter, capsid purification method, and transgene. DRG pathology was observed in 83% of NHP with administration of AAV to the CSF, and 32 % of NHP via the intravenous (IV) route. We show that dose and age at injection significantly affected the severity while sex had no impact. DRG pathology was absent at acute time-points (i.e., ≤14 days), similar from 1 to 5 months post-injection, and less severe after 6 months. Vector purification method had no impact, and all capsids and promoters that we tested caused some DRG pathology. The data presented here from 5 different capsids, 5 different promoters, and 20 different transgenes suggest that DRG pathology is almost universal after AAV gene therapy in nonclinical studies using NHP. None of the animals receiving a therapeutic transgene displayed any clinical signs. Incorporation of sensitive techniques such as nerve conduction velocities can show modifications in a minority of animals that correlate with the severity of peripheral nerve axonopathy. Monitoring of sensory neuropathies in human CNS trials and high dose IV studies seems prudent to determine if clinically meaningful DRG pathology occurs.

### Introduction

Gene therapy using recombinant adeno-associated virus (AAV) has been linked with histopathological findings in sensory neurons of dorsal root ganglia (DRG) in preclinical studies using nonhuman primates (J. Hordeaux, et al. Mol Ther Methods Clin Dev 10, 68-78, 2018; J. Hordeaux, et al. Mol Ther Methods Clin Dev 10, 79-88, 2018) and pigs (C. Hinderer, et al. Hum Gene Ther, 2018). The pathology manifests as mononuclear cell infiltrates and sensory neuron degeneration within the DRG in addition to secondary axonopathy which affects both the central axon of dorsal spinal cord tracts and peripheral axon of peripheral nerves (FIG. 14). DRG pathology or toxicity has been reported in nonclinical studies using AAV administration into the cerebrospinal fluid (CSF) (J. Hordeaux, et al. Mol Ther Methods Clin Dev 10, 68-78, 2018; J. Hordeaux, et al. Mol Ther Methods Clin Dev 10, 79-88, 2018; B. A. Perez, et al. Brain Sci 10, 2020) or systemic high dose administration to target the central nervous system (CNS) (C. Hinderer, et al. Hum Gene Ther, 2018). Animals remained asymptomatic in most of the studies in which pathology was minimal to moderate. More severe pathology and overt toxicity involving progressive proprioceptive deficits and ataxia were observed in a study of piglets within 14 days of high dose intravenous (IV) vector injection (C. Hinderer, et al. Hum Gene Ther, 2018). Immune suppression using a combination of mycophenolate mofetil and rapamycin did not eliminate the histopathological findings in NHP (J. Hordeaux, et al. Mol Ther Methods Clin Dev 10, 68-78, 2018; J. Hordeaux, et al. Mol Ther Methods Clin Dev 10, 79-88, 2018). The significance of DRG pathology in human clinical trials is unknown. Because most of the NHP studies have low number of animals and limited opportunity for statistical analysis, we aggregated data from 33 studies and conducted a meta-analysis on a total of 256 macaques to look for the effect of route of administration, dose, time course, study conduct, age of the animals, sex, capsid, promoter, capsid purification method, and transgene.

### Materials and Methods

### Data Availability Statement

Aggregated data are presented with experimental details provided except for the specific transgenes which are proprietary to the sponsors that funded the work.

### Animals

237 rhesus macaques and 19 cynomolgus macaques from 33 studies were included in this meta-analysis. All animal procedures were approved by the Institutional Animal Care and Use Committee of the University of Pennsylvania. Rhesus macaques (Macaca mulatta) or Cynomolgus macaques (Macaca fascicularis) were procured from Covance Research Products, Inc. (Alice, TX), Primgen/Prelabs Primates (Hines, IL), MD Anderson (Bastrop, TX), or were donated. Animals were housed in an Association for Assessment and Accreditation of Laboratory Animal Care (AAALAC) International-accredited Nonhuman Primate Research Program facility at the University of Pennsylvania or at Children’s Hospital of Philadelphia in stainless steel squeeze-back cages. Animals received varied enrichments such as food treats, visual and auditory stimuli, manipulatives, and social interactions.

### Test or Control Article Administration

For CSF administration, NHP received vectors diluted in sterile artificial CSF (vehicle) injected into the cisterna magna, under fluoroscopic guidance as previously described (N. Katz, et la. Hum Gene Ther Methods 29, 212-219, 2018). Lumbar puncture was performed under fluoroscopic guidance in anesthetized animals. After inserting a spinal needle into the L4-5 or L5-6 space, we confirmed placement by CSF return and/or by injecting up to 1 mL of contrast material (Iohexol 180). For intravenous administration, a catheter was placed in the saphenous vein and vector diluted in sterile 1x Dulbecco’s phosphate-buffered saline.

### Nerve Conduction Velocity Testing

Animals were sedated with a combination of ketamine/dexmedetomidine and placed in lateral or dorsal recumbency on a procedure table, with heat packs to maintain body temperature. The stimulator probe was positioned over the median nerve with the cathode closest to the recording site, and two needle electrodes inserted subcutaneously on digit II at the level of the distal phalanx (reference electrode) and proximal phalanx (recording electrode), while the ground electrode was placed proximal to the stimulating probe (cathode). A pediatric stimulator delivered the stimulus that we increased in a stepwise fashion until the peak amplitude response was reached. Up to 10 maximal stimuli were averaged and reported for the median nerve. The distance (cm) from the recording site to the stimulation cathode was measured and used to calculate the conduction velocity. Both the conduction velocity and the average of the sensory nerve action potential (SNAP) amplitude were reported.

### Vectors

For research studies, AAV vectors were produced and titrated by the Penn Vector Core as described previously (M. Lock et al. Hum Gene Ther 21, 1259-1271, 2010; M. Lock, et al. Hum Gene Ther Methods 25, 115-125, 2014). Briefly, HEK293 cells were triple-transfected and the culture supernatant was harvested, concentrated, and purified with an iodixanol gradient. For Good Laboratory Practice (GLP)-compliant toxicology studies, vector was also produced by triple-transfection of HEK293 cells and purified by affinity chromatography using a POROS™ CaptureSelect™ AAV9 resin (Thermo Fisher Scientific, Waltham, MA) as previously described (J. Hordeaux, et al. Mol Ther Methods Clin Dev 10, 79-88, 2018).

### Histopathology

For the majority of studies, a board-certified veterinary pathologist, initially blinded to test article/treatment groups, established severity scores defined as 0 for absence of lesion, 1 for minimal (<10%), 2 as mild (10-25%), 3 for moderate (25-50%), 4 for marked (50-95%), and 5 for severe (>95%). These scores were based on microscopic evaluation of hematoxylin and eosin (H&E)-stained tissues in which % represents the proportion of tissue affected by the lesion in an average high-power field. For all GLP and some non-GLP studies, peer-review was completed by an external board-certified veterinary pathologist. Severity scores from DRG degeneration and dorsal axonopathy of the spinal cord were established from cervical, thoracic, and lumbar segments; however, the number of sections evaluated varied across studies. In some studies, scores were assigned to individual sections of DRG and spinal cord when multiple tissue sections were present on a slide for a given segment; these were averaged for a single representative score. We consider spinal cord axonopathy as a better indicator of DRG pathology as it represents the collation of axons coming from all the DRG. We define DRG pathology as histopathological findings within the DRG cell bodies and spinal cord or spinal cord alone throughout this manuscript. Peripheral nerve axonopathy grades were established based on evaluation of the median (proximal and/or distal), radial, ulnar, sciatic (proximal and/or distal), peroneal, tibial, and/or sural nerves. When evaluation was performed on the proximal and distal median nerve, the proximal segment corresponded to the portion of nerve from the brachial plexus to the elbow and the distal segment corresponded to the portion of nerve from the elbow to the palm of the hand. When present, a severity score was given for periaxonal (i.e., endoneurial) fibrosis in peripheral nerves. For studies when peripheral nerves were evaluated bilaterally, axonopathy and periaxonal scores were averaged for each nerve.

### Data Extraction

The raw data including pathology scores and all pertinent study information were extracted from study files and aggregated in a single Excel spreadsheet. Two persons independently extracted and sorted the scores based on pre-determined search criteria to generate graphs and perform statistics. In case of discrepancy between the extracted outputs, consensus was reached upon collegial quality control.

### Statistics

For each parameter (i.e., age at injection, capsid, route of administration, time course, promoter, sex, vector purification method, and dose), we carried out comparisons of the pathology score between each pair of groups for each DRG or SC segment (i.e., cervical, thoracic and lumbar), using Wilcoxon rank-sum test with function “wilcox.test” within the R Program (version 3.5.0; https://cran.r-project.org).We then calculated combined p-values from the 3 comparisons for overall DRG or SC inter-group comparisons using Fisher’s method with function “sumlog” in the “metap” package in R. Statistical significance was assessed on combined p-value at the 0.05 level.

### Results

### DRG Pathology Assessment

We developed a method to accurately evaluate and score lesions to DRG neurons based on neuro-anatomy and systematic evaluation of neurons and their corresponding axons. Neuronal cell bodies of primary sensory neurons are ovoid swellings at the base of each spinal dorsal root in the subarachnoid space located within the DRG. DRG neurons are pseudo-unipolar with one peripheral branch extending into the peripheral nerve and one central branch ascending dorsally in the spinal cord white matter tracts (FIG. 14). It is our experience that neuronal degeneration does not affect DRG uniformly, meaning multiple DRG from cervical, thoracic, and lumbar regions need to be collected to provide a representative sample. Pathology in the DRG manifests as mononuclear cell infiltration involving mononuclear inflammatory cells and proliferating resident satellite cells, with neuronal degeneration becoming visible at a later stage (FIG. 14, A1 circles). Secondary to neuronal cell body injury is axonal degeneration (i.e., axonopathy) along DRG axonal projections in the nerve root (FIG. 14, B1), ascending dorsal tracts of the spinal cord (FIG. 14, C1), and peripheral nerves (FIG. 14, D1). Typical histopathological findings with the normal counterparts are pictured in FIG. 14, A1-D2; high magnification images of varying stages of DRG pathology are also shown. Early in the degenerative process, the neuronal cell bodies appear relatively normal with only proliferating satellite cells in addition to microglial cells and infiltrating mononuclear cells (neuronophagia, FIG. 14, panel E). As the lesions progress, the neuronal cell bodies exhibit evidence of degeneration (FIG. 14, panel F, vertical arrow) characterized by small, irregular- or angular-shaped cells with fading or absent nuclei and cytoplasmic hypereosinophilia. End-stage neuronal cell body degeneration (FIG. 14, panel G, circles) involves their complete obliteration (FIG. 14, panel G, star) by satellite cells, microglial cells and mononuclear cells. The severity of the histological findings in DRG and corresponding axons is graded based on the percentage of neurons or axons that are affected on an average high-power field: 0 as absence of lesion, 1 as minimal (<10%), 2 mild (10-25%), 3 moderate (25-50%), 4 marked (50-95%), and 5 severe (>95%). DRG represent a mosaic with an abundance of neurons being normal and only a minority of neurons showing degeneration on a given section. We consider spinal cord axonopathy as a better indicator of DRG pathology as it represents the collation of axons coming from all the DRG. We define DRG pathology as histopathological findings within the DRG cell bodies and spinal cord or spinal cord alone throughout this manuscript.

### Studies and Populations Characteristics

We aggregated data from 33 studies including 256 animals injected with AAV vectors or vehicle controls at the Gene Therapy Program at Penn from 2013 to 2020. A summary of these studies is provided in the table below.

NHP POPULATION AND STUDIES CHARACTERISTICS

| Parameter | Number of Animals | | | | | |
|---|---|---|---|---|---|---|
| Species | Rhesus (all) | Rhesus (male) | Rhesus (female) | Cynomolgus (all) | Cynomolgus (male) | Cynomolgus (female) |
| | 237 | 134 | 103 | 19 | 13 | 6 |
| Sex | Male | | | Female | | |
| | 147 | | | 109 | | |
| Age | Infant (1 month) | | Juvenile (1-3 years) | | Adult (>3 years) | |
| | 4 | | 58 | | 194 | |
| Route of Administration** | IV | ICM | LP | IV+ICM | | IM |
| | 27 | 213 | 6 | 4 | | 4 |
| Dose Range | IV | | | ICM and LP | | |
| | le13 GC/kg – 2e14 GC/kg | | | le12 GC – 3e14 GC | | |
| Capsids | 5 | | | | | |
| Promoters | 5 | | | | | |
| Transgenes | 20 | | | | | |
| Capsid Manufacturing for ICM/LP studies | GLP study (column-purified vector) | | | Non-GLP (iodixanol-purified vector) | | |
| | 101 | | | 103 | | |

**Un-injected n=2 (no pathology)

### Effect of Study Characteristics on Severity of DRG Pathology

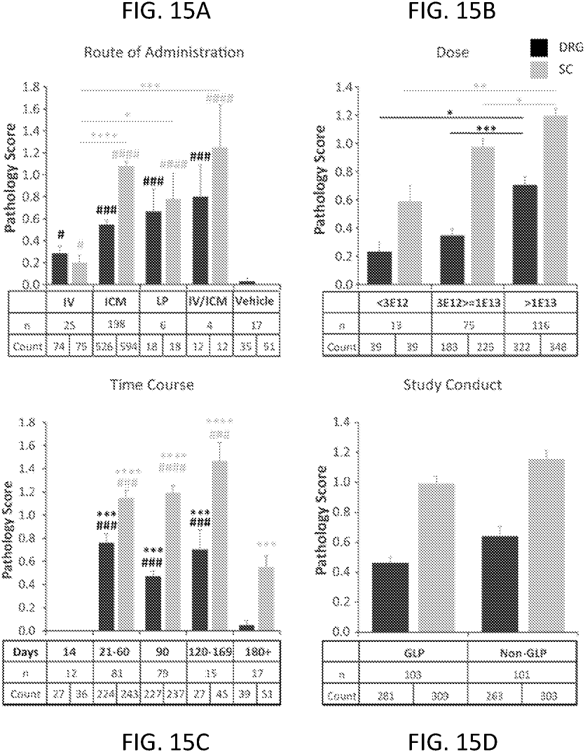

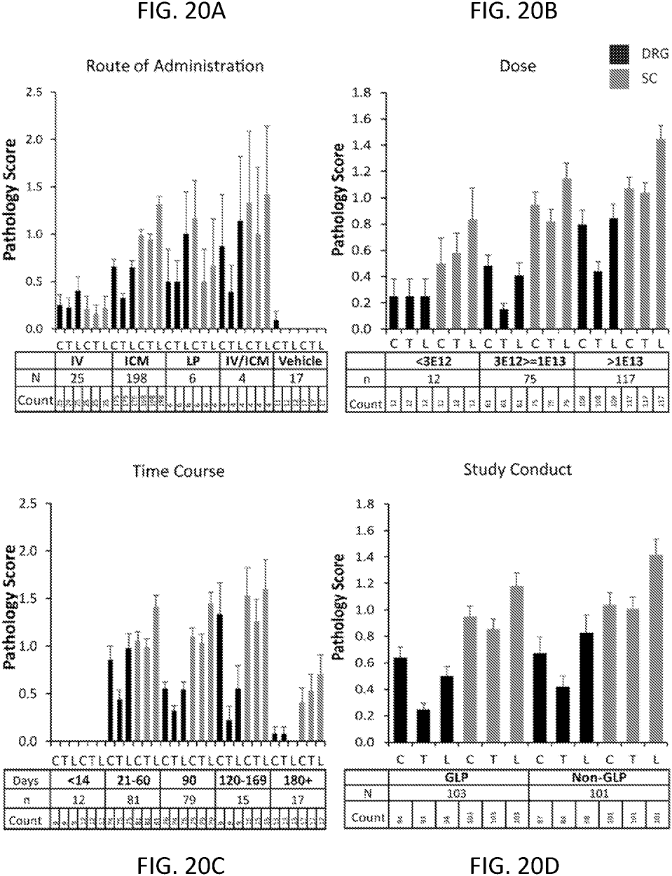

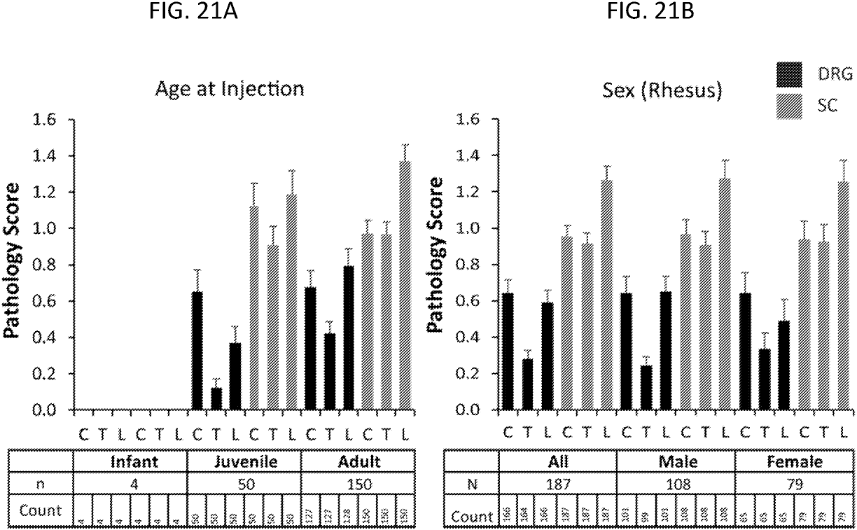

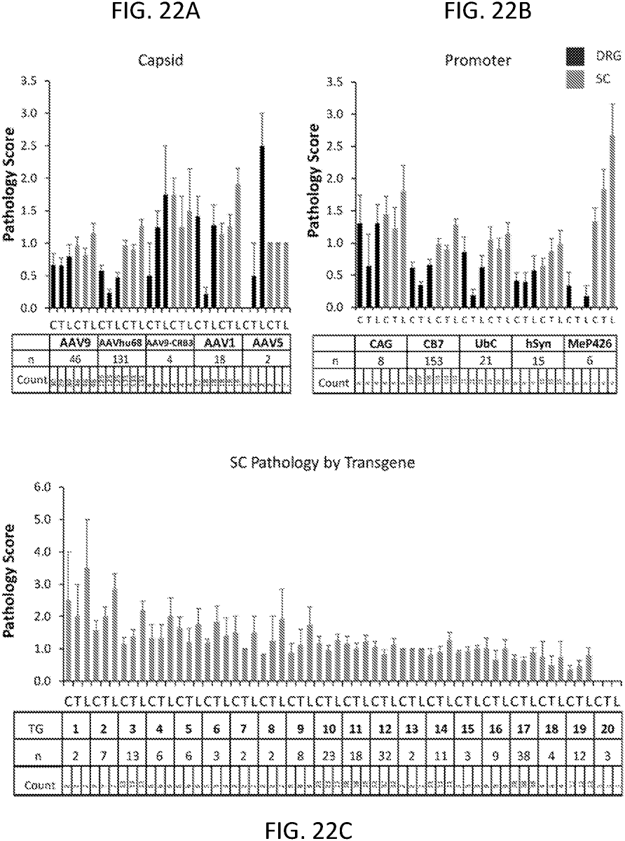

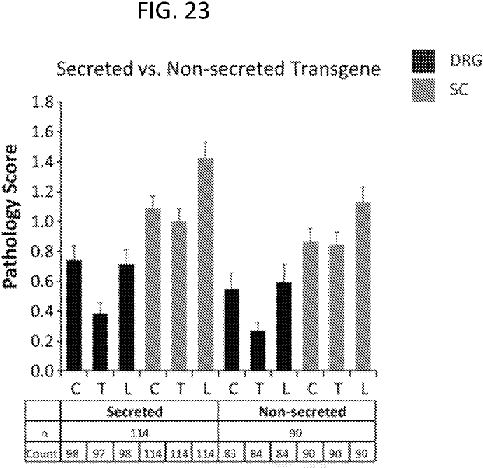

DRG pathology was observed in 83% of NHP that received AAV ICM or LP (170/205 animals), 32% of NHP for the IV route (8/25 animals), 100% for the combination ICM + IV (4/4 animals) and 0% for intramuscular (IM, 0/4 animals). Pathologists graded the DRG lesions based on severity score in DRG and their corresponding axons in spinal cord and peripheral nerves. Scores were obtained for each DRG and spinal region (cervical, thoracic, and lumbar). Average scores are depicted in FIGS. 15-17 with the data split by region presented in FIGS. 20-23. Severity in DRG was lower than in spinal cord because each spinal cord region groups the totality of axons coming from DRG, thus collating pathology scores from several DRG (FIG. 14). The study design parameters that significantly impacted the severity of the pathology were the route of administration (ROA), dose, and necropsy time point (FIGS. 15A - 15C). The compliance with GLP practice for nonclinical laboratory studies (as set in title 21 of the Code of Federal Regulations, part 58) did not impact the severity of the pathology (FIG. 15D). All the ROAs except IM led to significant pathology in both DRG and spinal cord when compared with vehicle controls (p=0.04 DRG and spinal cord, IV versus vehicle; p<0.001 DRG and p<0.0001 spinal cord, all other routes versus vehicle). ICM, LP, and ICM/IV were all similar and significantly worse than IV (IV vs ICM p<0.0001; IV vs LP p=0.02; IV vs ICM/IV p=0.0006- FIG. 15B). IM (not shown) did not lead to pathology (all score 0) and was similar to vehicle controls. For all the following analyses, we only considered animals with intra-CSF administration (i.e., ICM or LP). The 2 lower dose ranges (<3E+12 GC and 3E+12 - 1E+13 GC) were similar whereas the maximal dose range (> 1E+13 GC) led to significantly worse pathology scores than both the lowest (p=0.009, spinal cord) and the middle dose range (p= 0.001 DRG, p=0.05 spinal cord; FIG. 15B). The post-injection time point (i.e., when the necropsy was performed and tissues were analyzed) showed similar pathology severity between 21-60 days, 90 days, and 120-169 days. Pathology was not seen at the early (i.e., day 14) time point and the longer follow-ups greater than or equal to 180 days showed a significant reduction of severity compared to all other time-points (p<0.0001 spinal cord; p<0.0001 DRG D90; p<0.001 DRG other time-points).

### Effect of Animal Characteristics on Severity of DRG Pathology

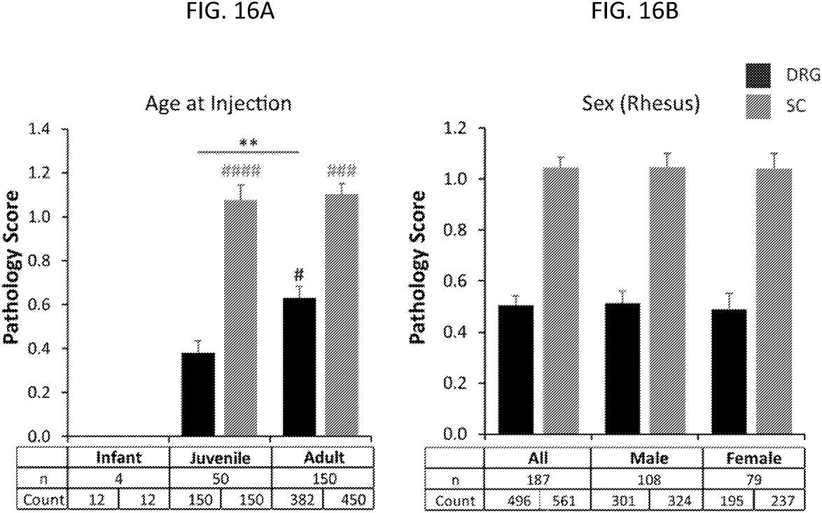

Age at vector administration had a significant effect on pathology severity. Juvenile animals had less severe DRG degeneration when compared with adults (p=0.003) but similar spinal cord axonopathy (FIG. 16A). The 4 animals treated as infants had no sign of DRG or spinal cord pathology as previously reported (J. Hordeaux et al. Hum Gene Ther 30, 957-966, 2019). This result needs to be interpreted with caution due to the small n and possible effect of the study endpoint (4 years post-injection). As shown on FIG. 15A - FIG. 15D, study duration has an impact on the severity of pathology and it is unclear whether the age at injection and/or study duration underpin the absence of pathology. Additionally, and importantly, sex had no effect on SC or DRG pathology (FIG. 16B).

### Effect of Vector Characteristics on Severity of DRG Pathology

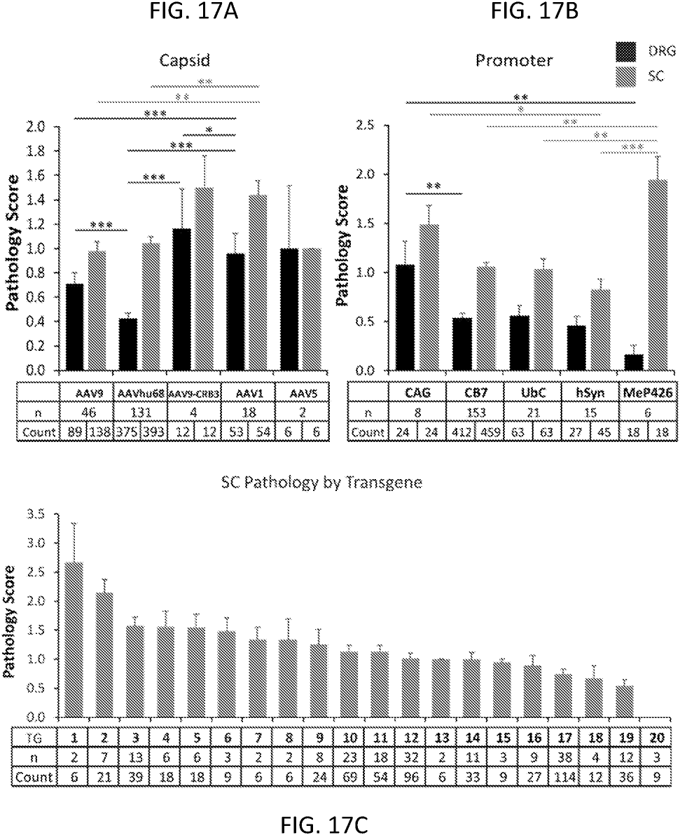

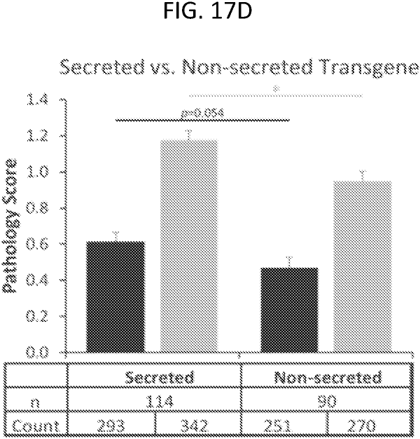

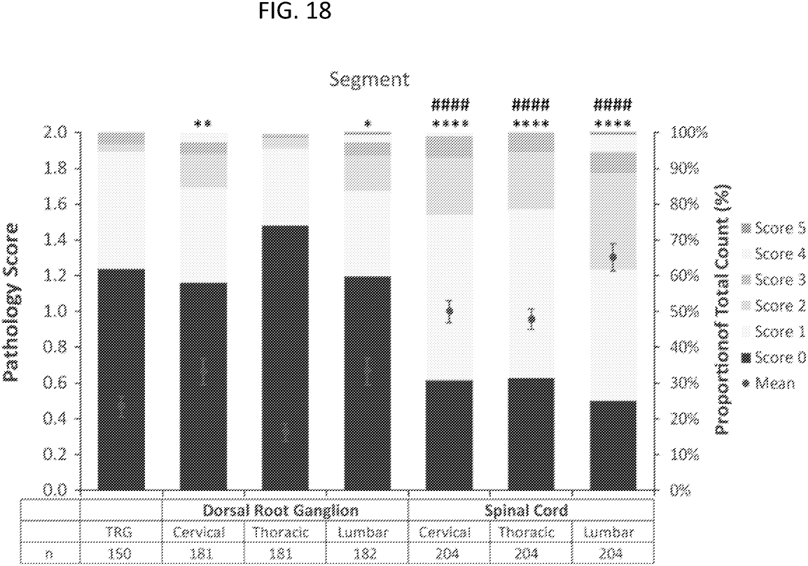

DRG neurodegeneration was present with all capsids, although there were some differences in severity amongst serotypes (FIG. 17A). Such variations, when restricted to DRG and not seen in spinal cord scores, may not be meaningful since the DRG represents a mosaic more susceptible to sampling artifact than the spinal cord cross-sections. Axonopathy and DRG scores were both significantly worse with AAV1 than AAVhu68 (p=0.01 spinal cord; p=0.0004 DRG) and with AAV1 than AAV9 (p=0.007 spinal cord and DRG - FIG. 17A). Ubiquitous promoters CAG, CB7, and UbC were all similar to each other while CAG led to worse axonopathy than hSyn (p=0.028) and MeP426 worse than CB7 (p=0.001), UbC (p=0.002) and hSyn (p=0.0003; FIG. 17B). We tested 20 different transgenes and all but one caused DRG pathology (FIG. 17C). Pathology severity varied greatly between transgenes (from 0.5 to 2.7 average spinal cord axonopathy score); pathology severity was 20-25% less for non-secreted transgenes compared to secreted transgenes (FIG. 17D; for DRG, secreted mean = 0.61, non-secreted mean = 0.47, p=0.05; for SC, secreted mean = 1.17, non-secreted mean = 0.94, p=0.02). Moreover, the purification method (i.e., iodixanol in non-GLP studies and column chromatography in GLP studies) did not impact the presence or severity of DRG pathology (FIG. 15D).

### Regional Severity, and Clinical Manifestations of DRG Pathology

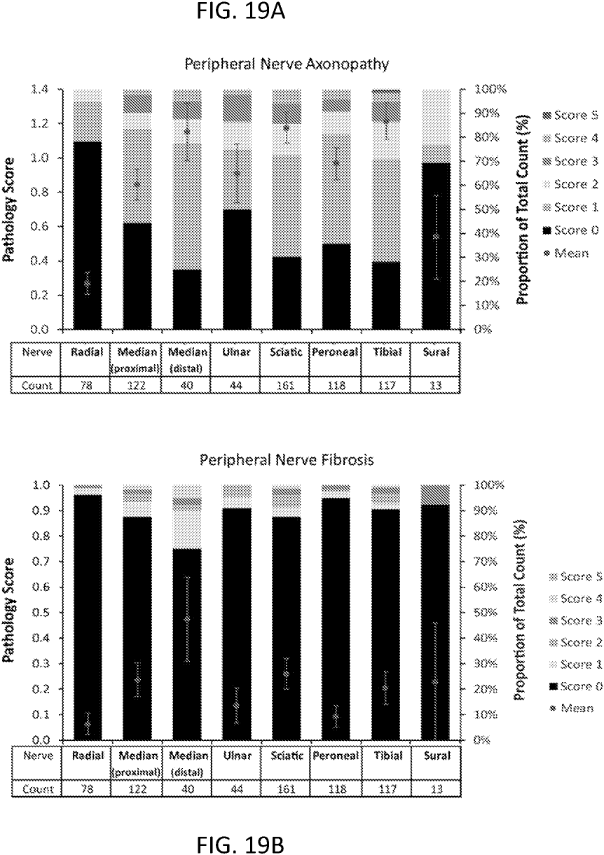

We assessed regional differences in pathology with respect to cervical, thoracic, and lumbar spine. The trigeminal nerve ganglion (TRG) was also analyzed as it represents a sensory ganglion with similar characteristics than the DRG located at the base of the skull inside the subarachnoid space. FIG. 18 shows a distribution of the actual pathology scores in each region as well as the average. TRG pathology was similar than cervical and lumbar DRG (non-significant) whereas the thoracic DRG had a less severe score (p=0.007). SC regional scores were all significantly worse than their corresponding DRG scores (p<0.0001), which is consistent SC collating axons from several DRG, meaning it comprises more lesions. The vast majority of sections have normal or low (grade 1) severity scores, with few grade 4 and very few grade 5 scores reported (grade 5 corresponds to 95 % or more of tissue surface affected by the lesion on an average high-power field) (FIG. 18). We performed neurological examination involving cage-side evaluation of mentation, posture, and gait, as well as restrained evaluation of cranial nerves, proprioception, motor strength, sensory function, and reflexes. Of 204 animals administered AAV ICM or LP, only 3 developed obvious pathology with clinical signs of ataxia and/or tremors. All 3 received vectors encoding GFP at doses >1E+13 GC and the pathology appeared 21 days post-injection. Nerve conduction velocities of the median nerve were recorded in 56 animals. Two developed a marked bilateral sensory amplitude reduction at 28 days post-injection that persisted until necropsy. This correlated with marked (grade 4 severity) axonopathy and endoneurial fibrosis in the median nerve but no obvious clinical sequelae. Most animals had low severity grades of axonopathy and fibrosis in peripheral nerves (FIG. 19).

### Discussion

DRG pathology and secondary axonopathy is minimal in the vast majority of our NHP studies and can be challenging to pick up for a non-trained eye. In our first GLP toxicology study evaluating an ICM AAV administration (J. Hordeaux, et al. Mol Ther Methods Clin Dev 10, 79-88, 2018), the CRO who performed the initial pathology assessment missed the lesion which was only caught by a peer-review pathologist experienced in neuropathology. Because neuronal degeneration is sparse and DRG are a mosaic of mostly normal neurons with few degenerative events on a given section, we found that multiple DRG need to be collected for robust histological analysis (we recommend at least 3 per spinal region). An easier way to detect and quantify DRG neuronal damage involves evaluating the secondary consequences of pathology in the cell body by assessing axon degeneration in the spinal cord; this is easier to detect and represents a collation of ascending fibers coming from multiple DRG.

When collecting and carefully analyzing the right tissues, we found some evidence of DRG pathology in 83% of NHP that received AAV ICM and 32% of NHP that received AAV IV. The IV doses showing pathology were as low as 1E+13 GC/kg, a dose currently evaluated in the clinics for several hemophilia trials (B. S. Doshi and V. R. Ther Adv Hematol 9, 273-293, 2018). Manufacturing purification method had no impact on pathology. All capsids and all promoters that we tested demonstrated some level of DRG pathology, which suggests that changing capsid or promoter is not a viable solution. Of relevance for nonclinical study design and for clinical translation, we found that dose and age at injection significantly affected the severity while sex had no impact. The aspect of our studies with the greatest impact on severity of pathology was the transgene, which is consistent with our hypothesis that transgene overexpression drives the early events which lead to degeneration. For most transgenes it was not possible to identify a No Observable Adverse Effect Level (NOAEL) above the Minimum Efficacy Dose (MED).

Time course is important to consider for study design as acute time-points (i.e., day 14 or below) do not show histopathology whereas longer studies (i.e., >180 days) tend to demonstrate less severe pathology, which suggests a lack of progression and possible partial remission over time. Our experience with health authorities has involved incorporating two necropsy time points - one after the onset of pathology (i.e., around 1 month) and another to show the pathology is not getting worse (i.e., 4 to 6 months). The four NHP infants that were dosed at 1-month of age included in our meta-analysis were remarkable for the absence of DRG and SC axon pathology despite good transgene expression levels when the animals were necropsied almost 4 years post-injection (J. Hordeaux et al. Hum Gene Ther 30, 957-966, 2019). This observation may suggest a more favorable safety profile when dosing infants or that acute pathology does not progress and in fact resolves; we did not have any early time point necropsies in this study.

None of the animals that received a therapeutic transgene (i.e., not a reporter gene such as GFP) demonstrated any clinical findings. In later studies, we incorporated routine monitoring of sensory neuron pathology through the use of nerve conduction velocity measurements. We did find NCV abnormalities in two animals associated with more severe peripheral nerves axonopathy and fibrosis (i.e., grade 4 severity) without evidence of clinical sequelae.

In summary, we show that pathology in DRGs is a consistent finding in virtually all NHPs studies when AAV vector is delivered into the subarachnoid space and in many studies when higher doses are administered systemically. Our meta-analysis is remarkable for a notable absence of any clinical sequelae. Careful analysis of other nonclinical studies in other species failed to show any evidence of DRG pathology, except in newborn pigs, suggesting NHPs are the best model for evaluating this potential pathology. Monitoring of sensory neuropathies in human CNS trials and high dose IV studies seems prudent to determine if clinically meaningful DRG pathology occurs.

### Example 11: Background Pathology in Control NHPs

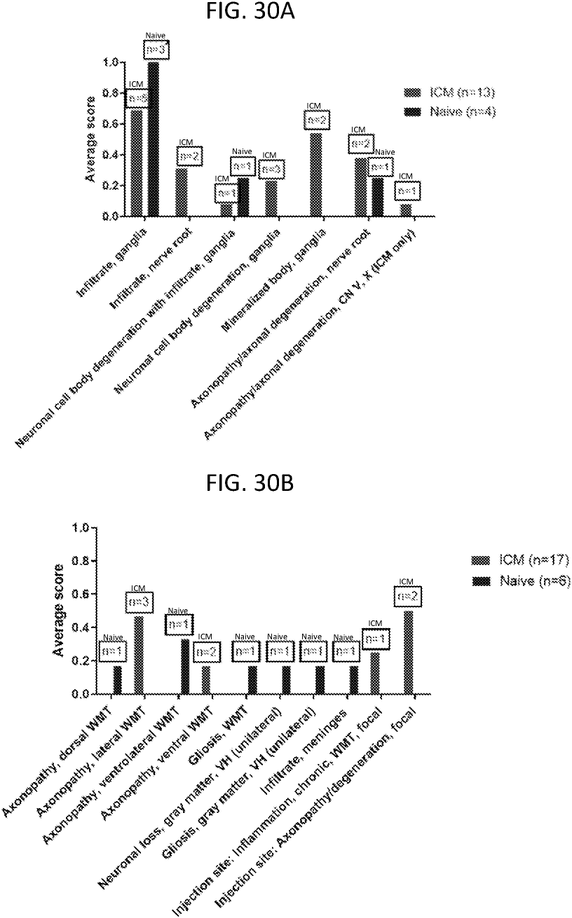

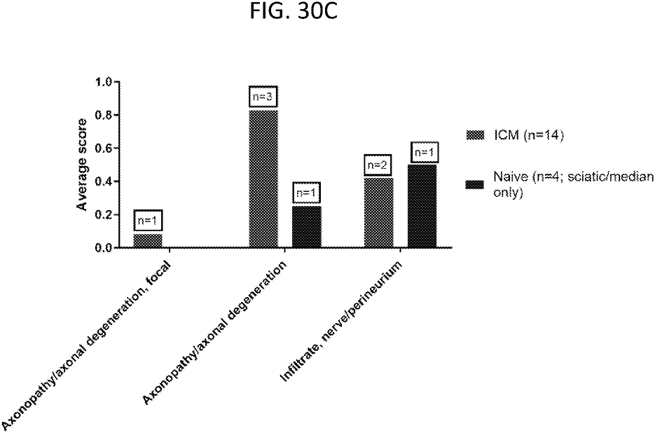

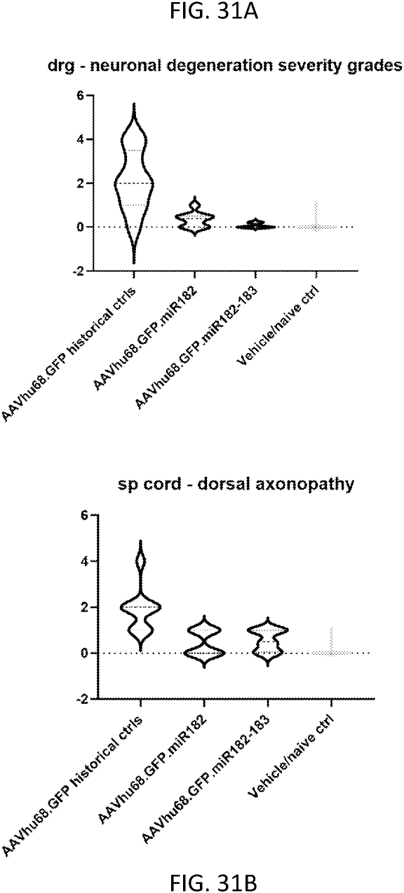

Analysis of results from previous studies was performed to determine background levels of pathology in tissues from historical control NHP animals (including naive control and vehicle-administered controls). In total, data were compiled from seven naive animals and 17 vehicle-administered (via ICM) controls. The results indicated that the incidence of pathology in the CNS and PNS was generally low. AAV-related DRG/TRG toxicity is typically associated with neuronal cell body degeneration with or without infiltrate. In the control animals, this finding was present in 4 out of 13 vehicle-administered animals and one out of four naive animals (FIG. 30A). In spinal cord, axonal degeneration (axonopathy) in the dorsal sensory white matter tracts was observed in 0 out of 17 vehicle-administered animals and one out of six naive animals (FIG. 30B). In peripheral nerves, axonal degeneration (axonopathy) in nerve sensory fibers was observed 3 out of 14 vehicle-administered animals and one out of four naive animals (FIG. 30C).

Where DRG toxicity was observed, the severity was scored up to 1 (minimal). Delivery of AAV vectors increased the incidence of grade 1 findings and was associated with findings greater than or equal to grade 2. Examples of scores from AAV-treated animals versus control animals are shown in FIG. 31A and FIG. 31B.

The findings suggest possible thresholds for assessing DRG toxicity. For example, a high level of confidence can be assigned to grade 2 findings in DRG, dorsal spinal cord, and/or peripheral nerves. Alternatively, or in addition, toxicity may be associated with an increased incidence of grade 1 findings.

### SEQUENCE LISTING FREE TEXT

The following information is provided for sequences containing free text under numeric identifier <223>.

| | |
|---|---|
| SEQ ID NO: (containing free text) | Free text under <223> |
| 1 | <223> miR-183 target |
| 2 | <223> mirR-96 target |
| 3 | <223> miR-182 target |
| 4 | <223> miR-145 target |
| 5 | <223> Spacer (i) |
| 6 | <223> Spacer (ii) |
| 7 | <223> spacer iii |
| 10 | <223> ITR.CB7.CI.eGFP.miR145.rBG.ITR |
| | <220> |
| | <221> repeat_region |
| | <222> (1)..(130) |
| | <223> 5′ - AAV2 - ITR |
| | <220> |
| | <221> misc_feature |
| | <222> (1)..(130) |
| | <223> 5′ - AAV2 - ITR |
| | <220> |
| | <221> promoter |
| | <222> (198)..(579) |
| | <223> CMV IE promoter |
| | <220> |
| | <221> promoter |
| | <222> (582)..(863) |
| | <223> CB promoter |
| | <220> |
| | <221> misc_feature |
| | <222> (1979)..(2698) |
| | <223> eGFP gene |
| | <220> |
| | <221> misc_feature |
| | <222> (2705)..(2727) |
| | <223> miR145 |
| | <220> |
| | <221> misc_feature |
| | <222> (2728)..(2731) |
| | <223> spacer |
| | <220> |
| | <221> misc_feature |
| | <222> (2732)..(2754) |
| | <223> miR145 |
| | <220> |
| | <221> misc_feature |
| | <222> (2755)..(2760) |
| | <223> spacer |
| | <220> |
| | <221> misc_feature |
| | <222> (2761)..(2783) |
| | <223> miR145 |
| | <220> |
| | <221> misc_feature |
| | <222> (2784)..(2789) |
| | <223> spacer |
| | <220> |
| | <221> misc_feature |
| | <222> (2790)..(2812) |
| | <223> miR145 |
| | <220> |
| | <221> misc_feature |
| | <222> (2981)..(3198) |
| | <223> 3′ ITR |
| 11 | <223> ITR.CB7.CI.eGFP.miR182.rGB.ITR |
| | <220> |
| | <221> misc_feature |
| | <222> (1)..(130) |
| | <223> 5′ ITR (AAV2) |
| | <220> |
| | <221> misc_feature |
| | <222> (198)..(579) |
| | <223> CMV IE promoter |
| | <220> |
| | <221> misc_feature |
| | <222> (582)..(863) |
| | <223> CB promoter |
| | <220> |
| | <221> misc_feature |
| | <222> (958)..(1930) |
| | <223> chicken beta-actin intron |
| | <220> |
| | <221> misc_feature |
| | <222> (1979)..(2698) |
| | <223> eGFP coding sequence |
| | <220> |
| | <221> misc_feature |
| | <222> (2705)..(2728) |
| | <223> miR182 |
| | <220> |
| | <221> misc_feature |
| | <222> (2729)..(2732) |
| | <223> spacer |
| | <220> |
| | <221> misc_feature |
| | <222> (2733)..(2756) |
| | <223> miR182 |
| | <220> |
| | <221> misc_feature |
| | <222> (2757)..(2760) |
| | <223> spacer |
| | <220> |
| | <221> misc_feature |
| | <222> (2763)..(2786) |
| | <223> miR182 |
| | <220> |
| | <221> misc_feature |
| | <222> (2787)..(2792) |
| | <223> spacer |
| | <220> |
| | <221> misc_feature |
| | <222> (2793)..(2816) |
| | <223> miR182 |
| | <220> |
| | <221> polyA_signal |
| | <222> (2858)..(2984) |
| | <220> |
| | <221> misc_feature |
| | <222> (3073)..(3202) |
| | <223> 3′ ITR |
| 12 | <223> ITR.CB7.eGFP.miRNA96.rBG.ITR |
| | <220> |
| | <221> misc_feature |
| | <222> (1979)..(2699) |
| | <223> eGFP coding sequence |
| | <220> |
| | <221> misc_feature |
| | <222> (2705)..(2727) |
| | <223> miR96 |
| | <220> |
| | <221> misc_feature |
| | <222> (2728)..(2731) |
| | <223> spacer |
| | <220> |
| | <221> misc_feature |
| | <222> (2732)..(2754) |
| | <223> miR96 |
| | <220> |
| | <221> misc_feature |
| | <222> (2755)..(2760) |
| | <223> spacer |
| | <220> |
| | <221> misc_feature |
| | <222> (2761)..(2783) |
| | <223> miR96 |
| | <220> |
| | <221> misc_feature |
| | <222> (2784)..(2789) |
| | <223> spacer |
| | <220> |
| | <221> misc_feature |
| | <222> (2790)..(2812) |
| | <223> miR96 |
| 13 | <223> ITR.CB7.CI.eGFP.miRNA183.rBG.ITR |
| | <220> |
| | <221> misc_feature |
| | <222> (1979)..(2698) |
| | <223> eGFP coding sequence |
| | <220> |
| | <221> misc_feature |
| | <222> (2705)..(2726) |
| | <223> miRNA183 |
| | <220> |
| | <221> misc_feature |
| | <222> (2727)..(2730) |
| | <223> spacer |
| | <220> |
| | <221> misc_feature |
| | <222> (2731)..(2752) |
| | <223> miRNA183 |
| | <220> |
| | <221> misc_feature |
| | <222> (2753)..(2758) |
| | <223> spacer |
| | <220> |
| | <221> misc_feature |
| | <222> (2781)..(2786) |
| | <223> spacer |
| | <220> |
| | <221> misc_feature |
| | <222> (2787)..(2808) |
| | <223> miRNA183 |
| 14 | <223> ITR.CB7.CI.hIDUAcoVl.rBG.ITR |
| 15 | <223> ITR.CB7.CI.hIDUAcoVl.miR183.ITR |
| | <220> |
| | <221> misc_feature |
| | <222> (1938)..(3908) |
| | <223> hIDUAcoV1 |
| | <220> |
| | <221> misc_feature |
| | <222> (3915)..(3936) |
| | <223> miRNA183 |
| | <220> |
| | <221> misc_feature |
| | <222> (3937)..(3940) |
| | <223> spacer |
| | <220> |
| | <221> misc_feature |
| | <222> (3941)..(3962) |
| | <223> miRNA183 |
| | <220> |
| | <221> misc_feature |
| | <222> (3963)..(3968) |
| | <223> spacer |
| | <220> |
| | <221> misc_feature |
| | <222> (3969)..(3990) |
| | <223> miRNA183 |
| | <220> |
| | <221> misc_feature |
| | <222> (3991)..(3996) |
| | <223> spacer |
| | <220> |
| | <221> misc_feature |
| | <222> (3997)..(4018) |
| | <223> miRNA183 |
| 20 | <223> CB7.CI.eGFP.miR183(lx).RBG |
| | <220> |
| | <221> repeat_region |
| | <222> (1)..(130) |
| | <223> AAV2 - 5′ITR |
| | <220> |
| | <221> enhancer |
| | <222> (198)..(579) |
| | <223> human cytomegalovirus (CMV) immediate early (IE) |
| | enhancer |
| | <220> |
| | <221> promoter |
| | <222> (582)..(862) |
| | <223> chicken beta-actin (CB) promoter |
| | <220> |
| | <221> Intron |
| | <222> (956)..(1928) |
| | <223> chicken beta-actin intron |
| | <220> |
| | <221> Misc |
| | <222> (1977)..(2696) |
| | <223> eGFP coding sequence |
| | <220> |
| | <221> misc_RNA |
| | <222> (2699)..(2720) |
| | <223> miR183 |
| | <220> |
| | <221> polyA _signal |
| | <222> (2762)..(2888) |
| | <223> rabbit globin polyA |
| | <220> |
| | <221> repeat region |
| | <222> (2977)..(3106) |
| | <223> AAV2 - 3′ ITR |
| 21 | <223> CB7.CI.eGFP.miRNA183(2x).RBG |
| | <220> |
| | <221> repeat_region |
| | <222> (1)..(130) |
| | <223> AAV 2 - 5′ITR |
| | <220> |
| | <221> enhancer |
| | <222> (198)..(579) |
| | <223> hCMV IE enhancer |
| | <220> |
| | <221> promoter |
| | <222> (582)..(862) |
| | <223> CB promoter |
| | <220> |
| | <221> Intron |
| | <222> (956)..(1928) |
| | <223> chicken beta-actin intron |
| | <220> |
| | <221> misc_feature |
| | <222> (1977)..(2696) |
| | <223> eGFP coding sequence |
| | <220> |
| | <221> misc_RNA |
| | <222> (2701)..(2722) |
| | <223> miRNA183 |
| | <220> |
| | <221> misc_feature |
| | <222> (2701)..(2722) |
| | <223> spacer |
| | <220> |
| | <221> misc_feature |
| | <222> (2723)..(2728) |
| | <223> spacer |
| | <220> |
| | <221> misc_RNA |
| | <222> (2729)..(2750) |
| | <223> miRNA183 |
| | <220> |
| | <221> polyA _signal |
| | <222> (2792)..(2916) |
| | <223> rabbit globin polyA |
| 22 | <223> CB7.CI.eGFP.miRNA183(3x).RBG |
| | <220> |
| | <221> repeat_region |
| | <222> (1)..(130) |
| | <223> AAV2 - 5′ITR |
| | <220> |
| | <221> enhancer |
| | <222> (198)..(579) |
| | <223> CMV IE enhancer |
| | <220> |
| | <221> promoter |
| | <222> (582)..(862) |
| | <223> CB promoter |
| | <220> |
| | <221> Intron |
| | <222> (956)..(1928) |
| | <223> chicken beta-actin promoter |
| | <220> |
| | <221> misc_feature |
| | <222> (1977)..(2696) |
| | <223> eGFP coding sequence |
| | <220> |
| | <221> misc_RNA |
| | <222> (2703)..(2724) |
| | <223> miRNA183 |
| | <220> |
| | <221> misc_feature |
| | <222> (2725)..(2728) |
| | <223> spacer |
| | <220> |
| | <221> misc_RNA |
| | <222> (2729)..(2750) |
| | <223> miRNA183 |
| | <220> |
| | <221> misc_feature |
| | <222> (2751)..(2756) |
| | <223> spacer |
| | <220> |
| | <221> misc_RNA |
| | <222> (2757)..(2778) |
| | <223> miRNA183 |
| | <220> |
| | <221> polyA _signal |
| | <222> (2819)..(2945) |
| | <223> rabbit globin polyA |
| | <220> |
| | <221> repeat region |
| | <222> (3034)..(3163) |
| 23 | <223> CB7.CI.eGFP.miRNA183.RBG |
| | <220> |
| | <221> repeat_region |
| | <222> (1)..(130) |
| | <223> AAV2 = 5′ITR |
| | <220> |
| | <221> enhancer |
| | <222> (198)..(579) |
| | <223> hCMV IE enhancer |
| | <220> |
| | <221> promoter |
| | <222> (582)..(862) |
| | <223> CB promoter |
| | <220> |
| | <221> Intron |
| | <222> (956)..(1926) |
| | <223> chicken beta-actin intron |
| | <220> |
| | <221> misc_feature |
| | <222> (1977)..(2696) |
| | <223> eGFP coding sequence |
| | <220> |
| | <221> misc_RNA |
| | <222> (2703)..(2724) |
| | <223> miRNA183 |
| | <220> |
| | <221> misc_feature |
| | <222> (2725)..(2729) |
| | <223> spacer |
| | <220> |
| | <221> misc_RNA |
| | <222> (2729)..(2750) |
| | <223> miRNA183 |
| | <220> |
| | <221> misc_RNA |
| | <222> (2757)..(2778) |
| | <223> miRNA183 |
| | <220> |
| | <221> misc_feature |
| | <222> (2779)..(2784) |
| | <223> spacer |
| | <220> |
| | <221> misc_RNA |
| | <222> (2785)..(2806) |
| | <223> miRNA183 |
| | <220> |
| | <221> polyA_signal |
| | <222> (2848)..(2974) |
| | <223> rabbit globin polyA |
| | <220> |
| | <221> repeat_region |
| | <222> (3063)..(3192) |
| | <223> AAV2 - 3′ ITR |
| 24 | <223> LAGLIDADG |
| 25 | <223> endonuclease recognition site |
| 26 | <223> pAA V.CB7.CI.eGFP.miRNA183(7X).RBG(p6031)-cl1 |
| | <220> |
| | <221> repeat_region |
| | <222> (1)..(130) |
| | <223> 5′ITR |
| | <220> |
| | <221> enhancer |
| | <222> (198)..(579) |
| | <223> CMV IE enhancer |
| | <220> |
| | <221> promoter |
| | <222> (582)..(862) |
| | <223> CB promoter |
| | <220> |
| | <221> TATA signal |
| | <222> (836)..(839) |
| | <223> TATA |
| | <220> |
| | <221> Intron |
| | <222> (956)..(1928) |
| | <223> chicken beta-actin intron |
| | <220> |
| | <221> misc_feature |
| | <222> (1977)..(2696) |
| | <223> eGFP |
| | <220> |
| | <221> misc_feature |
| | <222> (2729)..(2760) |
| | <223> miR183 |
| | <220> |
| | <221> misc_feature |
| | <222> (2757)..(2778) |
| | <223> miR183 |
| | <220> |
| | <221> misc_feature |
| | <222> (2785)..(2806) |
| | <223> miR183 |
| | <220> |
| | <221> misc_feature |
| | <222> (2815)..(2836) |
| | <223> miR183 |
| | <220> |
| | <221> misc_feature |
| | <222> (2858)..(2879) |
| | <223> miR183 |
| | <220> |
| | <221> misc_feature |
| | <222> (2884)..(2905) |
| | <223> miR183 |
| | <220> |
| | <221> polyA_signal |
| | <222> (2939)..(3065) |
| | <223> Rabbit globin poly A |
| | <220> |
| | <221> repeat_region |
| | <222> (3154)..(3283) |
| | <223> 3′ ITR |
| 27 | <223> pAAV.CB7.CI.eGFP.miRNA183(8X).RBG(P6015) |
| | <220> |
| | <221> repeat_region |
| | <222> (1)..(130) |
| | <223> 5′ITR |
| | <220> |
| | <221> enhancer |
| | <222> (198)..(579) |
| | <223> CMV IE enhancer |
| | <220> |
| | <221> promoter |
| | <222> (582)..(862) |
| | <223> CB promoter |
| | <220> |
| | <221> TATA signal |
| | <222> (836)..(839) |
| | <220> |
| | <221> Intron |
| | <222> (956)..(1928) |
| | <223> chicken beta-actin intron |
| | <220> |
| | <221> misc_feature |
| | <222> (1977)..(2696) |
| | <223> eGFP |
| | <220> |
| | <221> misc_feature |
| | <222> (2703)..(2724) |
| | <223> miR183 |
| | <220> |
| | <221> misc_feature |
| | <222> (2729)..(2750) |
| | <223> miR183 |
| | <220> |
| | <221> misc_feature |
| | <222> (2757)..(2778) |
| | <223> miR183 |
| | <220> |
| | <221> misc_feature |
| | <222> (2785)..(2806) |
| | <223> miR183 |
| | <220> |
| | <221> misc_feature |
| | <222> (2815)..(2836) |
| | <223> miR183 |
| | <220> |
| | <221> misc_feature |
| | <222> (2841)..(2862) |
| | <223> miR183 |
| | <220> |
| | <221> misc_feature |
| | <222> (2869)..(2890) |
| | <223> miR183 |
| | <220> |
| | <221> misc_feature |
| | <222> (2895)..(2916) |
| | <223> miR183 |
| | <220> |
| | <221> polyA_signal |
| | <222> (2950)..(3076) |
| | <223> Rabbit globin poly A |
| | <220> |
| | <221> repeat_region |
| | <222> (3165)..(3294) |
| | <223> 3′ITR |
| 28 | <223> pAAV.CB7.CI.eGFP.miR182.miR183.rBG |
| | <220> |
| | <221> repeat_region |
| | <222> (1)..(130) |
| | <223> 5′ITR |
| | <220> |
| | <221> enhancer |
| | <222> (198)..(579) |
| | <223> CMV IE enhancer |
| | <220> |
| | <221> promoter |
| | <222> (582)..(863) |
| | <223> CB promoter |
| | <220> |
| | <221> TATA signal |
| | <222> (836)..(839) |
| | <223> TATA |
| | <220> |
| | <221> misc_feature |
| | <222> (836)..(2698) |
| | <223> eGFP |
| | <220> |
| | <221> Intron |
| | <222> (958)..(1930) |
| | <220> |
| | <221> misc_feature |
| | <222> (2705)..(2728) |
| | <223> miR182 |
| | <220> |
| | <221> misc_feature |
| | <222> (2733)..(2756) |
| | <223> miR182 |
| | <220> |
| | <221> misc_feature |
| | <222> (2763)..(2786) |
| | <223> miR182 |
| | <220> |
| | <221> misc_feature |
| | <222> (2793)..(2816) |
| | <223> miR182 |
| | <220> |
| | <221> misc_feature |
| | <222> (2825)..(2846) |
| | <223> miR183 |
| | <220> |
| | <221> misc_feature |
| | <222> (2851)..(2872) |
| | <223> miR183 |
| | <220> |
| | <221> misc_feature |
| | <222> (2879)..(2900) |
| | <223> miR183 |
| | <220> |
| | <221> misc_feature |
| | <222> (2905)..(2926) |
| | <223> miR183 |
| | <220> |
| | <221> polyA_signal |
| | <222> (2960)..(3086) |
| | <223> Rabbit globin poly A |
| | <220> |
| | <221> repeat_region |
| | <222> (3175)..(3304) |
| | <223> 3′ ITR |

All publications cited in this specification are incorporated herein by reference in their entireties. PCT/US19/67872, filed Dec. 20, 2019, U.S. Provisional Pat. Application No. 62/783,956, filed Dec. 21, 2018, U.S. Provisional Pat. Application No. 62/924,970, filed Oct. 23, 2019, U.S. Provisional Pat. Application No. 62/934,915, filed Nov. 13, 2019, U.S. Provisional Pat. Application No. 62/972,4040, filed Feb. 10, 2020, U.S. Provisional Pat. Application No. 63/005,894, filed Apr. 6, 2020, U.S. Provisional Pat. Application No. 63/023,593, filed May 12, 2020, U.S. Provisional Pat. Application No. 63/038,488, filed Jun. 12, 2020, U.S. Provisional Pat. Application No. 63/043,562, filed Jun. 24, 2020, U.S. Provisional Pat. Application No. 63/079,299, filed Sep. 16, 2020, and U.S. Provisional Pat. Application No. 63/152,042, filed Feb. 22, 2021, are hereby incorporated by reference in their entireties. Similarly, the SEQ ID NOs referenced herein and appearing in the appended Sequence Listing (21-9594PCT_ST25.txt) are incorporated by reference. While the invention has been described with reference to particular embodiments, it will be appreciated that modifications can be made without departing from the spirit of the invention. Such modifications are intended to fall within the scope of the appended claims.

1. A recombinant AAV (rAAV) for delivery of a gene product to a patient in need thereof which specifically represses expression of the gene product in dorsal root ganglia (DRG), said rAAV comprising an AAV capsid having packaged therein a vector genome, wherein the vector genome comprises:
(a) a coding sequence for the gene product under the control of regulatory sequences that direct expression of the gene product in a cell containing the vector genome; and
(b) at least eight miR target sequences, wherein each target sequence is specific for miR-183 or miR-182, and wherein the at least eight miR target sequences are operably linked to the 3′ end of the coding sequence.

2. The rAAV according claim 1, wherein the at least eight miR target sequences comprise at least five, at least six, at least seven, or at least eight target sequences specific for miR-183.

3. The rAAV according claim 1, wherein the at least eight miR target sequences comprise at least five, at least six, at least seven, or at least eight target sequences specific for miR-182.

4. The rAAV according to any one of claims 1 to 3, wherein the at least eight miR target sequences comprise at least four target sequences specific for miR-183 and/or at least four target sequences specific for miR-182.

5. The rAAV according to claim 1, wherein the at least eight miR target sequences comprise four target sequences specific for miR-183 and four target sequences specific for miR-182.

6. The rAAV according to any one of claims 1 to 5, wherein the expression cassette comprises a 3′ UTR having at least eight miR target sequences.

7. The rAAV according to any one of claims 1 to 6, wherein the least eight miR target sequences are in a 3′ UTR that is 200 to 1200 nucleotides in length.

8. The rAAV according to any one of claims 1 to 7, wherein the at least eight miR target sequences are continuous or are separated by a spacer of 1 to 10 nucleotides, wherein the spacer is not a miRNA target sequence.

9. The rAAV according to any one of claims 1 to 8, wherein the 5′ end of the first of the at least eight miR target sequences is within 20 nucleotides from the 3′ end of the gene coding sequence.

10. The rAAV according to any one of claims 1 to 8, wherein the 5′ end of the first of the at least eight miR target sequences is at least 100 nucleotides from the 3′ end of the gene coding sequence.

11. The rAAV according to any one of claims 1 to 10, wherein the vector genome further comprises at least one target sequence specific for miR-183 or miR-182 in a 5′ UTR.

12. The rAAV according to any one of claims 1 to 11, wherein each of the at least eight target sequences comprises
(a) AGTGAATTCTACCAGTGCCATA (SEQ ID NO: 1); or
(b) AGTGTGAGTTCTACCATTGCCAAA (SEQ ID NO: 3).

13. The rAAV according to any one of claims 1 to 12, wherein the at least eight miR target sequences are continuous and not separated by a spacer.

14. The rAAV according to any one of claims 1 to 13, wherein each of the at least eight miR target sequences are separated by a spacer and each spacer is independently selected from one or more of (i) GGAT (SEQ ID NO:5); (ii) CACGTG (SEQ ID NO: 6); or (iii) GCATGC (SEQ ID NO: 7).

15. The rAAV according to any one of claims 1 to 14, wherein a spacer is located between each of the at least eight miR target sequences and 3′ to the first miRNA target sequence and/or 5′ to the last miR target sequence.

16. The rAAV according to any one of claim 1 to 15, wherein the vector genome comprises a tissue-specific promoter.

17. The rAAV according to any one of claim 1 to 16, wherein the vector genome comprises a central nervous system-specific promoter, a muscle-specific promoter, a cardiac-specific promoter, or a liver-specific promoter.

18. The rAAV according to any one of claims 1 to 15, wherein the vector genome comprises a constitutive promoter.

19. A composition for gene delivery which specifically represses expression of a gene product in dorsal root ganglia (DRG), comprising an expression cassette that is a nucleic acid sequence comprising:
(a) a coding sequence for the gene product under the control of regulatory sequences that direct expression of the gene product in a cell containing the expression cassette; and
(b) at least eight miR target sequences, wherein each target sequence is specific for miR-183 or miR-182, and wherein the at least eight miR target sequences are operably linked to the 3′ end of the coding sequence.

20. The composition according to claim 19, wherein the at least eight miR target sequences comprise at least five, at least six, at least seven, or at least eight target sequences specific for miR-183.

21. The composition according to claim 19, wherein the at least eight miR target sequences comprise at least five, at least six, at least seven, or at least eight target sequences specific for miR-182.

22. The composition according to any one of claims 19 to 21, wherein the at least eight miR target sequences comprise at least four target sequences specific for miR-183 and/or at least four target sequences specific for miR-182.

23. The composition according to any one of claims 19 to 22, wherein at least eight miR target sequences comprise four target sequences specific for miR-183 and four target sequences specific for miR-182.

24. The composition according to any one of claims 19 to 23, wherein the expression cassette comprises a 3′ UTR having at least eight miR target sequences.

25. The composition according to claim 24, wherein the 3′ UTR having at least eight miR target sequences is 200 to 1200 nucleotides in length.

26. The composition according to any one of claims 19 to 25, wherein the at least eight miR target sequences are continuous or are separated by a spacer of 1 to 10 nucleotides, wherein the spacer is not a miR target sequence.

27. The composition according to any one of claims 19 to 26, wherein the 5′ end of the first of the at least eight miR target sequences is within 20 nucleotides from the 3′ end of the gene coding sequence.

28. The composition according to any one of claims 19 to 26, wherein the 5′ end of the first of the at least eight miR target sequences is at least 100 nucleotides from the 3′ end of the gene coding sequence.

29. The composition according to any one of claims 19 to 28, wherein the expression cassette further comprises at least one target sequence specific for miR-183 or miR-182 in a 5′ UTR.

30. The composition according to any one of claims 19 to 29, wherein each of the at least eight target sequences comprises
(a) AGTGAATTCTACCAGTGCCATA (SEQ ID NO: 1); or
(b) AGTGTGAGTTCTACCATTGCCAAA (SEQ ID NO: 3).

31. The composition according to any one of claims 19 to 30, wherein the at least eight miR target sequences are continuous and not separated by spacers.

32. The composition according to any one of claims 19 to 30, wherein each of the at least eight miR target sequences are separated by a spacer and each spacer is independently selected from one or more of (i) GGAT (SEQ ID NO: 5); (ii) CACGTG (SEQ ID NO: 6); or (iii) GCATGC (SEQ ID NO: 7).

33. The composition according to any one of claims 26 to 30 or 32, wherein the spacers between each of the at least eight miRNA target sequences are the same.

34. The composition according to any one of claims 19 to 33, wherein the expression cassette is carried by a viral vector that is a recombinant parvovirus, a recombinant lentivirus, a recombinant retrovirus, or a recombinant adenovirus.

35. The composition according to any one of claims 19 to 33, wherein the expression cassette is carried by a non-viral vector that is naked DNA, naked RNA, an inorganic particle, a lipid particle, a polymer-based vector, or a chitosan-based formulation.

36. A pharmaceutical composition comprising the rAAV according to any one of claims 1 to 18 or the expression cassette according to any one of claims 19 to 35 and a formulation buffer suitable for delivery via intracerebroventricular, intrathecal, intracisternal, or intravenous injection.

37. A method for repressing expression of a gene product in DRG neurons in a patient, said method comprising delivering the rAAV according to any one of claims 1 to 18, the composition according to any one of claims 19 to 35, or the pharmaceutical composition according to claim 36 to the patient.

38. A method for modulating neuronal degeneration and/or decreasing secondary dorsal spinal cord axonal degeneration following intrathecal or systemic gene therapy administration to a patient, said method comprising delivering the rAAV according to any one of claims 1 to 18, the composition according to any one of claims 19 to 35, or the pharmaceutical composition according to claim 36 to the patient.

39. The rAAV according to any one of claims 1 to 18, the composition according to any one of claims 19 to 35, or the pharmaceutical composition according to claim 36 for use in gene delivery, wherein expression of the delivered gene product is repressed in DRG neurons of the patient.

40. Use the rAAV according to any one of claims 1 to 18, the composition according to any one of claims 19 to 35, or the pharmaceutical composition according to claim 36 for delivering a transgene to a patient, wherein expression of the delivered transgene is repressed in DRG neurons of the patient.
